(19) United States
(12) Patent Application Publication
(10) Pub. No.: US 2022/0284983 A1
(43) Pub. Date: Sep. 8, 2022

(54) METHODS OF IDENTIFYING CIS-REGULATORY ELEMENTS AND USES THEREOF

(71) Applicant: UNIVERSITY HEALTH NETWORK, Toronto (CA)

(72) Inventors: Mathieu Lupien, Toronto (CA); Benjamin Haibe-Kains, Toronto (CA); Seyed Ali Madani Tonekaboni, Toronto (CA)

(21) Appl. No.: 17/631,247

(22) PCT Filed: Aug. 3, 2020
(86) PCT No.: PCT/CA2020/051062

§ 371 Date: Jan. 28, 2022

Related U.S. Application Data
(60) Provisional application No. 62/882,173, filed on Aug. 2, 2019.

(51) Int. Cl. G16B 15/30; G16B 20/30; G16B 20/00; G16B 40/30

(52) U.S. Cl. CPC G16B 15/30; G16B 20/30; G16B 20/00; G16B 40/30

(57) ABSTRACT

The present disclosure relates to the development of methods for identifying cis-regulatory elements. Also disclosed herein are various methods including for example determining the tissue of origin of a biological sample, prognosis of a patient diagnosed with a cancer and their response to treatments.

### FIELD

The present disclosure relates to methods for identifying clusters of genomic regions such as clusters of cis-regulatory elements (COREs) and large organized chromatin lysine (K) domains (LOCKs) and their uses. Also disclosed herein are various methods based on said clusters of genomic regions for determining the tissue of origin of a biological sample, prognosis of a patient diagnosed with a cancer, stemness of a cell and other methods.

### INTRODUCTION

Over 98% of the human genome consists of sequences lying outside of gene coding regions that harbor functional features, including cis-regulatory elements (CREs) important in defining cellular identity by establishing lineage-specific gene expression profiles (Lupien et al. 2008; Heintzman et al. 2009; Ernst et al. 2011) and large organized chromatin Lysine (K) (LOCK) modifications. CREs such as enhancers, promoters and anchors of chromatin interactions, are predicted to cover 20-40% of the noncoding sequences of the human genome (Kellis et al. 2014). Current methods to annotate CREs in biological samples include ChIP-seq for histone modifications (e.g., H3K4me1, H3K4me3 and H3K27ac) (Heintzman et al. 2007, 2009; Lupien et al. 2008; Ernst and Kellis 2010), chromatin binding protein (e.g., MED1, P300, CTCF and ZNF143) (Heintzman et al. 2007; Bailey et al. 2015) or through chromatin accessibility assays (e.g., DNase-seq and ATAC-seq) (Thurman et al. 2012; Buenrostro et al. 2013).

CREs are unevenly distributed across the genome. High CRE density such as those reported as super-enhancers or stretch-enhancers are significantly associated to cell identity and are bound by transcription regulators with higher intensity than individual CREs (Whyte et al. 2013; Hnisz et al. 2013; Dowen et al. 2014; Boeva et al. 2017). High-density CRE regions from cancer cells have been found to lie proximal to oncogenic driver genes (Lo\acute{v}en et al. 2013; Northcott et al. 2014; Chipumuro et al. 2014; Kron et al. 2017).

The diverse phenotypic identities of each cell type found in multicellular organisms are encoded by lineage-specific biochemically active genomic features, such as transcribed genes¹, active transposable elements², anchors of chromatin interactions setting distal boundaries for loop extrusions defining the three-dimensional genome², DNA-to-lamin points of contact linking discrete genomic regions to the nuclear lamina⁴, Early Replicating Control Elements (ERCE)⁵, and other cis-regulatory elements such as promoters and enhancers⁶⁻¹⁰. Clusters of nucleosomes with post-translationally modified histone lysine residues define large organized chromatin lysine (K) domains (LOCKs) associated with inactive domains when consisting of dimethylated lysine 9 on histone 3 (H3K9me2)¹⁵ or H3K27me3¹⁶.

The uneven distribution of CREs and LOCKs across the genome has made their identification and characterization technically and biologically challenging. Methods for identifying and annotating high density CREs in super enhancers include ROSE and ROSE 2.

### SUMMARY

Described herein is a methodology termed CREAM (Clustering of genomic REgions Analysis Method) relying on chromatin accessibility profiles to identify Clusters Of cis-Regulatory Elements (COREs) in any cell type. CREAM is a computational method relying on unsupervised machine learning that for example takes into account the distribution of distances between CREs in a given biological sample to systematically identify clusters of genomic regions such as COREs, consisting of at least two individual CREs. Threshold for the stitching distance between individual CREs within each CORE is learned for each data (e.g. derived from a biological sample), thereby adjusting the stitching distance to the nature of the distance distribution across individual CREs within a given data CREAM provides for example a systematic way for the identification of COREs, outperforming other widely used CRE annotations, such as super-enhancers. CREAM is demonstrated to identify COREs enriched in proximity of highly expressed and essential genes compared to individual CREs. COREs are shown to be bound with high signal intensity by master transcription regulators in for example a cell type-specific manner in contrast to individual CREs. In addition, the enrichment of CTCF and the cohesin complex is revealed at a subset of COREs populating the boundaries of Topologically Associated Domain (TADs) illustrating the utility of COREs in studying the three-dimensional structure of the genome. Finally, the clinical value of identifying COREs in tumour samples to discriminate the cancer type and the biological underpinning specific to each sample is demonstrated herein.

Among the various aspects of the present disclosure is a method of determining a clusters of genomic regions signature in a biological sample. The clusters of genomic regions can be clusters of cis-Regulatory Elements (CORE) or clusters of Large organized chromatin lysine (K) (LOCK) regions.

Accordingly, an aspect is a method of determining a clusters of genomic regions (COGRs) optionally a Clusters of cis-Regulatory Elements (COREs) signature, referred to as a COGR signature or CORE signature, in a biological sample, the method comprising:
obtaining a chromatin profile of genomic DNA of the biological sample; identifying two or more individual cis-regulatory elements (CREs) in the chromatin profile; and locating one or more COGRs optionally COREs, to generate a COGR signature, optionally a CORE signature, comprising the steps of:
i) grouping different numbers of neighboring individual CREs throughout the genome and categorizing the groups according to order (O) which is the number of neighboring individual CREs in the groups;
ii) identifying a maximum window size (MWS) by estimating a distribution of window sizes for each order O based on a maximum distance between the individual CREs in all groups of that order O within the chromatin profile and calculating the MWS according to the following:
   a) MWS=Q1(log(WS))−1.5*IQ(log(WS)), where Q1(log(WS)) and IQ(log(WS)) are first quartile and interquartile distributions of window sizes of that order O, respectively; or
   b) MWS=Q1(log(WS_{normalized}))−1.5*IQ(log(WS_{normalized})) where

$$W {S}_{normalized} = max ( \frac{\begin{matrix}distance between two consecutive \\ elements in the group of CREs\end{matrix}}{\begin{matrix}average size of the two consecutive \\ elements in the group of CREs\end{matrix}} ) ;$$

iii) identifying a maximum O (Omax), defined as a value of given order O at which an increase in the given order O does not result in an increase in the MWS for the given order O;
iv) identifying potential COGRs, optionally potential COREs, by calling groups of CREs of a particular order O with a window size less than the MWS for the particular order O as the potential COGRs, optionally the potential COREs, for each order O from Omax to O=2; and
v) for each order O from O=2 to Omax, calculating the change in (MWS-median(WS))/median(WS), where WS is a distribution of maximum distance between individual CREs within the potential COGRs, optionally the potential COREs, of that order and filtering out lower order potential COGRs, optionally potential CORES up to a point where (MWS-median(WS))/median(WS) decreases with increasing order O, and any remaining potential COGRs, optionally potential COREs, are identified as actual COGRs, optionally actual COREs, and included in the COGR signature, optionally the CORE signature.

In one embodiment, the method is for determining Clusters of cis-Regulatory Elements (CORE) signature in a biological sample, the method comprising:

obtaining a chromatin accessibility profile of genomic DNA of the biological sample;

identifying two or more individual cis-regulatory elements (CREs) in the chromatin accessibility profile; and

locating one or more Clusters of cis-Regulatory Elements (COREs) to generate a CORE signature, the locating comprising the steps of:
i) grouping different numbers of neighboring individual CREs throughout the genome and categorizing the groups according to order (O);
ii) identifying a maximum window size (MWS) by estimating a distribution of window sizes for each order O based on a maximum distance between individual CREs in all groups of that order O within the genome and calculating the MWS according to the following:
   a) MWS=Q1(log(WS))−1.5IQ(log(WS)), where Q1(log(WS)) and IQ(log(WS)) are the first quartile and interquartile distributions of window sizes, respectively, or
   b) MWS=Q1(log(WS_{normalized}))−1.5*/Q(log(WS_{normalized})) where

$$W {S}_{normalized} = max ( \frac{\begin{matrix}distance between two consecutive \\ elements in the group of CREs\end{matrix}}{\begin{matrix}average size of the two consecutive \\ elements in the group of CREs\end{matrix}} ) ;$$

iii) identifying a maximum O (Omax), defined as the value of a given order O at which an increase in the order O does not result in an increase in the MWS or the given order O compared to a previous order O;
iv) identifying COREs by calling groups of neighboring CREs with a window size less than the MWS as potential COREs for each order O from order Omax to O=2; and
v) for each order O from O=2 to Omax, calculating the change in (MWS-median(WS))/median(WS) using the potential COREs, where WS is a distribution of maximum distance between individual CREs within COREs of that order O and filtering out all COREs for lower orders up to the point where (MWS-median(WS))/median(WS) decreases with increasing order O, and any remaining potential COGRs, optionally potential COREs, are identified as actual COGRs, optionally actual COREs, and included in the COGR signature, optionally the CORE signature.

In one embodiment, the chromatin profile is a chromatin accessibility profile, optionally an ATAC-seq or a DNAse-seq, and the COGR signature is a CORE signature.

In another embodiment, the chromatin profile a histone modification profile, optionally ChIP-seq profiles of a histone modification selected from H3K4me1, H3K4me3, H3K27ac, H3K9me3, H3K27me3 and H3K36me3 and the COGR signature a LOCKs signature, the method further comprising repeating steps i) to v) until the parameter defined by the equation

$$Relative sum - \frac{sum of coverage of LOCKs by individual elements}{sum of total genome coverage of LOCKs}$$

starts oscillations of >5%.

In an embodiment, the method is performed for a plurality of biological samples, each of the biological samples having an associated or determined phenotype of a plurality of phenotypes, the method further comprising identifying one or more COGRs of the COGR signatures to be associated with one of the phenotypes of the plurality of phenotypes, the one or more COGRs and optionally included in one or more of COGR signature standards.

In an embodiment, the identifying one or more COGRs of the COGR signatures comprises:
assessing each COGR of each COGR signature to build a univariate predictive model of outcome;
ranking each COGR according a phenotype prediction, and
optionally generating a multivariate prediction model using two or more ranked COGRs,
   wherein one or more of the identified COGRs with phenotype prediction above a selected threshold is selected to provide the COGR signature standard.

Various analyses can be performed after identifying a COGR signature.

In one embodiment, the method comprises identifying genomic structure and/or one or more genes, optionally gene transcriptional start sites, within 25 kb upstream or downstream of one or more of the COGR signature COGRs or a CRE within the COGR.

COGR signature standards associated with a phenotype of interest can be determined and used to classify unknown samples and for example prognose patients or identify tumor or origin of a patient sample, for biomarker discovery and the like.

Accordingly, another aspect is a method of for identifying a biomarker associated with a selected phenotype, the method comprising:

determining a COGR signature as described herein, optionally a CORE signature, for a plurality of biological samples, each of the biological samples having an associated or determined phenotype for one of a plurality of phenotypes;

assessing each COGR of each COGR signature to build a univariate predictive model of outcome;

ranking each COGR according a phenotype prediction, and

optionally generating a multivariate prediction model using two or more ranked COGRs;

wherein one or more of the COGRs of the COGR signature with phenotype prediction above a selected threshold is the identified phenotype biomarker and optionally provides a COGR signature standard.

In an embodiment, the phenotype is drug sensitivity, stemness of a biological sample, or enrichment for stem cells in a tumor sample and the methods involve identifying biomarkers associated with the phenotype.

Another aspect of the disclosure is a method of determining a cell or tissue of origin or a differentiation state of cells of a biological sample, the method comprising determining a COGR signature, optionally a CORE signature of the biological sample as described herein, comparing the COGR signature to one or more COGR signature standards from one or more cells or tissues of known origin or differentiation state of cells; and identifying the cell or tissue or origin and/or the differentiation state of the biological sample according to the COGR signature standard most similar to the COGR signature.

In an embodiment, the plurality of cells or tissues of known origin and/or differentiation states comprises at least 4 cells or tissues of known origin and/or differentiation states per cell or tissue type or differentiation state.

Accordingly, another aspect is a method for identifying if a tumor sample is enriched for cancer stem cells, the method comprising

determining a COGR signature of a biological sample of known tumor type, as described herein;

assessing a similarity of the COGR signature of the biological sample with at least one COGR signature standard of a tumor sample known to be stem cell enriched and at least one COGR signature standard of a tumor sample known not to be stem cell enriched;

determining any difference between the similarity to the at least one COGR signature standard known to be stem cell enriched and the at least one COGR signature standard known not to be stem cell enriched; and

assigning a score indicative of the stemness of the biological sample.

In an embodiment, the stemness score is assigned by determining an average Jaccard similarity of the at least one stem cell enriched COGR signature standard (A) and an average Jaccard similarity of the at least one non-stem cell enriched COGR signature standard (B), wherein the stemness score is A-B.

In an embodiment, the biological sample is a leukemia sample.

A further aspect is a method of identifying a drug target, the method comprising:

determining a COGR signature as described herein, particularly a CORE signature, for a plurality of biological samples, each of the biological samples having an associated or determined phenotype for one of a plurality of phenotypes, preferably one of two phenotypes, optionally the two phenotypes including leukemia stem cells+ and leukemia stem cells-;

assessing each COGR of each COGR signature to build a predictive model of each phenotype;

ranking each COGR according to phenotype prediction to identify top COGRs,

identifying individual CREs within one or more of the top COGRs that are specific for a selected phenotype of the plurality of phenotypes,

determining a COGR signature as described herein for cells genetically deleted of one or more of the individual CREs determined to be present in cells of the selected phenotype;

determining if the genetically deleted cells have an altered or non-selected phenotype; and

identifying one or more of the ranked COREs as potential drug targets if the COGR signature of the genetically deleted cells is more similar to a non-selected phenotype than the selected phenotype.

In an embodiment, the method further comprises identifying individual CREs in one or more of the ranked COREs specific to the selected phenotype, genetically modifying one or more of the individual CREs in a cell population corresponding to the selected phenotype and assessing if the genetic modification changes the cell population so that it is more similar to the non-selected phenotype than the selected phenotype.

Another aspect is a method for identifying a prognostic biomarker, the method comprising:

determining a COGR signature as described herein, particularly a CORE signature, for a plurality of tumor samples of a tumor type, each of the tumor samples having associated outcome data;

assessing each COGR of each COGR signature to build a univariate predictive model of outcome;

ranking each COGR according to a risk association prediction, and

optionally generating a multivariate prediction model using two or more ranked COGRs,

wherein one or more of the identified COGRs with risk prediction above a selected threshold is the prognostic biomarker, and optionally provides a COGR signature standard.

A method of determining the prognosis of a patient diagnosed with a cancer, the method comprising determining a COGR signature, optionally a CORE signature, of a biological sample previously acquired from the patient as described herein, wherein the biological sample is a tumor sample, comparing the COGR signature, optionally the CORE signature of the biological sample to one or more COGR signature standards, optionally CORE signature standards, associated with an outcome, and providing the patient with a prognosis according to the associated outcome of the COGR signature standard, optionally the CORE signature standard, with a greatest similarity to the COGR signature of the biological sample.

In some embodiments ATAC-seq is used to provide the chromatin accessibility profile for the biological sample. In some embodiments DNase-seq or Faire-seq is used to provide the chromatin accessibility profile for the biological sample. Other datasets can also be used.

As demonstrated herein, a number of COREs have been identified that are prognostic. In some embodiments the patient has been diagnosed with lung adenocarcinoma and the prognosis is determined to be poor, optionally a three year survival rate of about zero when a CORE is detected at chr10:14532486-14612373 or chr12:56751131-56775057 or good when said CORE at chr10:14532486-14612373 or chr12:56751131-56775057 is absent. In some embodiments the prognosis is determined to be very poor, optionally a one year survival rate of about zero when a CORE is detected at chr10:14532486-14612373 and chr12:56751131-56775057.

In some embodiments the patient has been diagnosed with colon adenocarcinoma and a CORE is identified at one or more of the following chromosomal locations: a) chr14:55050826-55052359; b) chr10:93417251-93483337; c) chr12:27243328-27348144; d) chr13:27169061-27175204; e) chr17:28318276-28385918; f) chr19:43518020-43535912; and/or g) chr2:74548755-74577205. In some embodiments the prognosis is determined to be poor, optionally a five year survival rate of about zero when a CORE is identified at least at one of a) or b) and good when said CORE at a) or b) is not detected. In some embodiments the prognosis is determined to be poor, optionally a four year survival rate of about zero when a CORE is identified at two or more of a) through g), optionally, when a CORE is identified at: a) and c); c) and e); d) and e); or f) and g) and good when said COREs are not detected. In some embodiments the prognosis is determined to be very poor, optionally a two year survival rate of about zero when a CORE is identified at three or more of a) through g), optionally a CORE is identified at: a), b), c), d), e) and f); a), d), e) and f); a), b), c), f) and g); or b), c), d), e) and g) and good when said COREs are not detected.

In some embodiments, the patient has been diagnosed with kidney renal papillary cell carcinoma, and the prognosis is determined to be good if a CORE is identified at more than two of the following chromosomal locations: 1) chr6_43469265_43523300; 2) chr1_192805761-192814841; 3) chr1_227944063-_227949006; 4) chr12_57450441_-57463071; 5) chr16_70379118_-70382380; 6) chr17_63768370_-63786036; 7)chr19-5677033-_5721143; 8)chr20-46346737-_46368831; 9)chr8-96260662-_96268917; 10) chr8_141338513-_141447436; and/or 11) chrX_143632935-_143636339.

In some embodiments, the patient has been diagnosed with stomach adenocarcinoma, and the prognosis is determined to be very poor, optionally a one year survival rate of about zero, when a CORE is identified at one or none of the following chromosomal locations: 1) chr13_113140862_113217081; 2) chr2_237858279_237905713; and/or 3) chr20_3845512_3847548.

In some embodiments, the patient has been diagnosed with liver hepatocellular carcinoma, and the prognosis is determined to be very poor, optionally a two year survival rate of about zero, when a CORE is identified at one or more of the following chromosomal locations: 1) chr1:167600169-167606030; 2) chr1:235646218-235651336; 3) chr10:59173772-59182122; 4) chr11:121652910-121657125; 5) chr2:102020545-102071073; 6) chr20:19958226-20019574; 7) chr5:10351451-10355436; 8) chr5:60699338-60700881; 9) chr5:75034328-75056067; 10) chr5:78636611-78649820; 11) chr5:78971924-78986100; 12) chr6:28349674-28357446; and 13) chr8:49909523-49924044.

In some embodiments, the patient has been diagnosed with lung squamous cell carcinoma, and the prognosis is determined to be poor, optionally a three year survival rate of zero, when a CORE is identified at one or more of the following chromosomal locations: 1) chr19:17605694-17607218′ and 2) chr1:113388501-113394601.

A further aspect is a method of assessing if a patient, model system, like a cell line or mouse model, with breast cancer is likely to respond to a treatment comprising PD98059 or Floxuridine, the method comprising:

determining a CORE signature of a biological sample previously acquired from the patient as described herein, wherein the biological sample is a breast cancer sample, comparing the CORE signature to one or more CORE signature standards having an associated drug sensitivity, the CORE signature standard for PD98059 drug sensitivity comprising a biomarker in Table 2, optionally chr11:694436-876984 and the CORE signature standard for floxuridine drug sensitivity comprising a biomarker in Table 3 optionally chr4:99547495-99584170, and identifying the patient outcome according to the associated drug sensitivity of the CORE signature standard with the greatest similarity.

A further aspect is a method of monitoring disease progression, the method comprising:

determining a first COGR signature, optionally a first CORE signature, of a biological sample previously acquired from the patient as described herein,

determining a subsequent COGR signature, optionally a subsequent CORE signature, of a subsequent biological sample previously acquired from the patient as described herein

comparing the first COGR signature and the subsequent COGR signature and one or more COGR signature standards each associated with an outcome, and

determining if the subsequent signature COGR signature is the same or more similar to a good outcome COGR signature standard than is the first COGR signature, indicating a lack of progression or determining if the first signature COGR signature is the same or more similar to a good outcome COGR signature standard than is the subsequent COGR signature, indicating disease progression.

In one embodiment, the subsequent biological sample is obtained after the patient has started treatment.

In another embodiment, the method comprises treating the patient or changing the treatment, if the patient is progressing.

Also provided is a system comprising:

a memory having program code stored thereon; and

a processor that is operatively coupled to the memory, wherein the processor is configured to perform one or more methods described herein when at least some of the program code is executed by the processor.

A computer readable medium having program code stored thereon that configures a processor, when executing at least some of the program code, to perform one or more methods described herein.

The preceding section is provided by way of example only and is not intended to be limiting on the scope of the present disclosure and appended claims. For example, the various aspects and embodiments of the disclosure may be utilized in numerous combinations, all of which are expressly contemplated by the present description. These additional advantages, objects and embodiments are expressly included within the scope of the present disclosure. The publications and other materials used herein to illuminate the background of the disclosure, and in particular cases, to provide additional details respecting the practice, are incorporated by reference, and for convenience are listed in the appended reference section.

### DRAWINGS

Further objects, features and advantages of the disclosure will become apparent from the following detailed description taken in conjunction with the accompanying figures showing illustrative embodiments of the disclosure, in which:

FIG. 1A shows a block diagram of an example embodiment of a genomic regions analysis system for various analyses based on clusters of genomic regions.

FIG. 1B shows a schematic representation of the five main steps of Clustering of genomic REgions Analysis Method (CREAM): Step 1) CREAM identifies all groups of 2, 3, 4 and more neighboring CREs. The total number of CREs in a group defines its “Order”; Step 2) Identification of the maximum window size (MWS) between two neighboring CREs in group for each Order. The MWS corresponds to the greatest distance allowed between two neighboring CREs in a given cluster; Step 3) identification of maximum Order limit of COREs from a given dataset; Step 4) CORE reporting according to the criteria set in step 3 from the highest to the lowest Order; Step 5) Identify minimum Order limit of COREs based on the identified COREs in Step 4.

FIG. 1C shows a flow chart of an example embodiment of a biomarker discovery method in accordance with the teachings herein.

FIG. 1D shows a flow chart of an example embodiment of a prognostic method in accordance with the teachings herein.

FIG. 1E shows a flow chart of an example embodiment of a stemness cell identifier method in accordance with the teachings herein.

FIG. 1F shows a flow chart of an example embodiment of a genomic or gene analysis method in accordance with the teachings herein.

FIG. 1G shows a flow chart of an example embodiment of a drug target identifier method in accordance with the teachings herein.

FIG. 1H shows a flow chart of an example embodiment of a tissue of origin identifier method in accordance with the teachings herein.

FIG. 2 shows a comparison of genomic characteristics of the COREs identified by CREAM versus individual CREs in the GM12878, K562, and H1-hESC cell lines. (A) Distribution of DNase I signal intensity in individual CREs and COREs (signal per base-pair). (B) Expression level of genes in 10 kb proximity of individual CREs or COREs (**** corresponds to p-value<0.0001). (C) Median expression of genes according to distance to the closest individual CRE or CORE. (D) Volcano plot of significance (FDR) and effect size (essentiality score) of genes in proximity of CREAM-identified COREs in K562 cell line (dark: significant fold change, light: insignificant fold change). (E) Essentiality score from K562, KBM-7, Jiyoye, and Raji cell line for genes proximal (∓10 kb) to COREs identified by CREAM in K562 cell line (**** corresponds to p-value<0.0001 using Wilcoxon signed-rank test). (F) Expression level of essential genes associated with individual CREs versus COREs (** corresponds to p-value<0.01).

FIG. 3 shows transcription regulator binding intensity in individual CREs and COREs. (A) Enrichment of transcription regulator binding intensity from ChIP-seq data in COREs identified by CREAM versus individual CREs from DNase-seq in the GM12878, K562 or H1-hESC cell lines. Volcano plots represent −log 10(FDR) versus log 2(fold change [FC]) in ChIP-seq signal intensities (each dot is one transcription regulator) (dark: significant fold change, light: insignificant fold change). The barplots show how many transcription regulators (TRs) have significant higher signal intensity in COREs or individual CREs (FDR<0.001 and log 2(FC)>1). Fold change (FC) is defined as the ratio between the average signal per base pair in COREs versus individual CREs. (B) Distribution of ChIP-seq signal intensity at COREs and individual CREs for TCF3 and EBF1 as examples of master transcription regulators in GM12878, for GABP and CREB1 as examples of master transcription regulators in the K562 cell line, and for NANOG and CMYC as examples of master transcription regulators in the H1-hESC cell line. (C) Examples of genomic regions with COREs (with different coverage) occupied by transcription regulators presented in (B).

FIG. 4 shows the arrangement of COREs and individual CREs with respect to TAD boundaries. (A) Schematic representation of a TAD boundaries and intra-TAD regions (25 kb Hi-C resolution). (B) Comparison of fraction of COREs and individual CREs from DNAse-seq that lie at TAD boundaries with increasing distance from TAD boundary cutoffs in the GM12878 and K562 cell lines. (C) Enrichment of transcription regulator (TR) binding intensities within COREs over individual CREs that lie in proximity of TAD boundaries (±10 kb) versus COREs and CREs farther away from TAD boundaries (intra-TAD elements) in the GM12878 or K562 cell line. (D) Enrichment of TR binding intensity in COREs proximal to TAD boundaries (±10 kb) versus intra-TAD domains. (E) Fraction of HCTs and individual TR binding regions at TAD boundaries (∓10 kb). The total number of individual binding regions for each TR in the GM12878 and K562 cell lines is also reported. (F) Examples of HCTs for CTCF, RAD21, SMC3, and ZNF143 at the TAD boundary for the MYC and BCL6 genes (10 kb Hi-C resolution).

FIG. 5 shows a comparison of CREAM-identified COREs and super-enhancers of the GM12878, K562 and H1-hESC cell lines. (A) Similarity of COREs and super-enhancers based on their genomic loci overlap. (B) Top 5 enriched biological pathways using genes in 10 kb proximity of the identified COREs and super-enhancers in each one of the GM12878, K562 and H1-hESC cell lines. (C) Percentage of COREs and super-enhancers containing 2 or more individual CREs. (D) Expression of genes in 10 kb proximity of both COREs and super-enhancers or exclusively in proximity of COREs or super-enhancers. (E) Enrichment of essential genes among genes in proximity of both COREs and super-enhancers or exclusively in proximity of COREs or super-enhancers. (F) Enrichment of transcription regulator binding intensity from ChIP-seq data in COREs identified by CREAM versus super-enhancers. Volcano plots represent −log 10(FDR) versus log 2(fold change [FC]) in ChIP-seq signal intensities (each dot is one transcription regulator) (dark: significant fold change, light: insignificant fold change). The barplots show how many transcription regulators (TRs) have significant higher signal intensity in COREs or super-enhancers (FDR<0.001 and log 2(FC)>1). Fold change (FC) is defined as the ratio between the average signal per base pair in COREs versus super-enhancers. (G) Distribution of ChIP-seq signal intensity of CTCF at COREs and super-enhancers in 10 kb proximity of TAD boundaries.

FIG. 6 shows the biology of COREs in human tumor samples. (A) Balanced accuracy for classification of TCGA tumor samples based on their tissue of origin using CREAM-identified COREs. (B) Enrichment of highly expressed genes in proximity (∓10 kb) of CREAM-identified COREs versus individual CREs for TCGA tumor samples. Boxplots show the null distribution corresponding to expression of randomly selected genes and each dot corresponds to the expression of proximal genes to COREs for each tumor sample in TCGA. (C) Enrichment of hallmark gene sets relying on genes in proximity (∓10 kb) of COREs versus genes in proximity (∓10 kb) individual CREs for TCGA tumor samples.

FIG. 7 shows Specificity of COREs to the cell types in ENCODE. (A) Number of COREs identified by CREAM for each cell or tissue previously profiled by the ENCODE project for DNase-seq. (B) Positive correlation in the number of COREs with the number of individual CRE per sample. (C) Independence in the fraction of CREs called within COREs versus the number of individual CRE per sample. (D) Relation between median width of COREs and the number of individual CRE per sample. (E) Heatmap of similarities based on CREAM-identified COREs across the ENCODE project cells or tissue samples based on Jaccard index of overlap in COREs.

FIG. 8 shows (A) Percentage of COREs shared between different percentage of cell lines with available DNaseI profiles in The ENCODE Project Consortium (B) Fraction of COREs overlapped with active TSS in GM12878, K562 and H1-hESC cell lines.

FIG. 9 shows expression of genes in 10 kb and 25 kb proximity of individual CREs or COREs categorized as either overlapping or distal to active TSS in GM12878, K562 and H1-hESC cell lines.

FIG. 10 shows (A) Enrichment of transcription regulator binding intensity from ChIP-seq data in COREs excluding the CRE-free gaps compared to individual CREs. (B) Enrichment of transcription regulator binding intensity from ChIP-seq data in COREs versus individual CREs categorized as either overlapping or distal to active TSS in GM12878, K562 and H1-hESC cell lines.

FIG. 11 shows (A) Permutation test assessing the significance of the enrichment of COREs at TAD boundaries across a range of distance cutoffs around TAD boundaries (0-500 kb). (B) Enrichment of COREs and individual CREs at TAD boundaries in HeLa, HMEC, HUVEC, and NHEK cells. (C) Relationship between the percentage of TR-CORE at TAD boundary and the % GC content of the individual CREs within the COREs.

FIG. 12 shows prognostic signatures and their corresponding survival rate in Kaplan-Meier plot signatures relying on identified COREs by CREAM using ATAC-seq profiles of patient tumor samples, with lung and colon adenocarcinoma.

FIG. 13 shows prognostic signatures and their corresponding survival rate in Kaplan-Meier plot signatures relying on identified COREs by CREAM using ATAC-seq profiles of patient tumor samples with kidney renal papillary cell carcinoma, stomach adenocarcinoma, liver hepatocellular carcinoma and lung squamous cell carcinoma.

FIG. 14 shows genomic coverage of LOCKs discriminates primitive from differentiated cell types. A) Genomic coverage of LOCKs identified using H3K4me1, H3K4me3, H3K27ac, H3K9me3, H3K27me3 and H3K36me3 histone modifications profiles across 13 primitive, 9 ES-derived and 77 differentiated cell types. B) Comparison of genomic coverage by LOCKs and individual regions (Ind. elements) post-translationally modified with histone marks in 9 primitive and 77 differentiated cell types (ES-derived excluded). Each dot corresponds to one cell type investigated.

FIGS. 15A and 15B shows LOCKs are predictive of cell identity according to tissue of origin. Unsupervised clustering of 13 primitive, 9 ES-derived and 77 differentiated cell types according to the similarity in the genomic localization of LOCKs from H3K4me1 (A), H3K4me3 (A), H3K27ac (A), H3K9me3 (B), H3K27me3 (B) and H3K36me3 (B) histone modifications.

FIG. 16 shows genes associated with LOCKs enrich for pathways related to the tissue of origin. Gene set enrichment analysis (GSEA) on the collection of genes found within LOCKs of various histone modifications reveals pathways of relevance to stem, hematopoiesis, brain, muscle, digestive and epithelial tissues.

FIG. 17 shows bivalent LOCKs are observed only in primitive cell types and associated with genes repressed in primitive cell types. A) Expression level of genes in proximity of LOCKs versus individual regions (Ind. elements) marked by H3K4me1, H3K4me3, H3K27ac, H3K9me3 or H3K27me3 in the primitive H1-hESC versus differentiated GM12878 and K562 cell types. B) Quantification of H3K27me3 ChIP-seq signal overlap in LOCKs of active histone modifications (H3K4me1, H3K4me3 and H3K27ac) for H1-hESC, GM12878 and K562 cell types. The signal is normalized (divided by median) to the H3K27me3 ChIP-seq signal overlapped in the Ind. elements of the corresponding profiles in each cell line. C) Gene set enrichment analysis (GSEA) reporting the enrichment of pathways in active LOCKs (H3K4me1, H3K4me3 and H3K27ac) and LOCKs of H3K27me3 repressive mark associated or not with elevate H3K27me3 signal in the H1-hESC primitive cell type.

FIG. 18 shows bivalent LOCKs in primitive cells are enriched at TAD boundaries and bound by regulators of chromatin interactions. A) Enrichment of LOCKs of active marks with low or high H3K27me3 ChIP-seq signal at TAD boundaries defined within H1-hESC, GM12878 or K562 cell types (−log 10(FDR)). B) Enrichment of regulators of chromatin interactions (CTCF, RAD21, ZNF143, YY1) in H3K4me1 LOCKs with low or high H3K27me3 ChIP-seq signal in H1-hESC, GM12878 and K562 cell types (−log 10(FDR)). C) Case example of a bivalent LOCK at a TAD boundary in H1-hESC cells on the chromosome 16q22.1 locus. The ChIP-seq signal intensity for H3K4me1, H3K27me3 and regulators of chromatin interactions are shown. D) Comparison of median of H3K4me1, H3K27me3 and H3K9me3 ChIP-seq signal overlap of H1-hESC, GM12878 and K562 cell lines on the H3K4me1 bivalent LOCKs (with high H3K27me3 signal overlap) in H1-hESC. The signal is normalized to the depth of ChIP-seq profiles and size of the LOCKs. In the heatmap, every value for each ChIP-seq profile is also divided by the maximum value of that profile across the cell lines.

FIG. 19 shows A) Similarity of hematopoietic samples identified using CREAM-identified COREs. B) Similarity of Leukaemia samples enriched with stem cells (LSC+) and differentiated cells (LSC−) using CREAM-identified COREs.

FIG. 20 shows A) COREs with highest performance in identification of LSC+ compared to LSC− samples using CREAM based drug target discovery. B) Percentage of LSC+ and LSC− samples with the top CORE identified in (B). C) Percentage of LSC+ and LSC− samples with the individual elements (peaks) within the top CORE identified in (B). D) Experimental validation regarding validity of top COREs in (B) as potential drug target for LSC+ samples. The shown bars are percentage of LSC+ cells remained after knocking out CRE3 and CRE6 as the individual elements, within the top CORE identified in (B), discriminating LSC+ and LSC− samples.

FIG. 21 shows the top biomarkers identified using COREs as biomarkers of response to PD98059 and Floxuridine tested on 16 breast cancer cell lines in the GRAY dataset with available ATAC-seq profiles.

### DESCRIPTION OF VARIOUS EMBODIMENTS

The following is a detailed description provided to aid those skilled in the art in practicing the present disclosure. Unless otherwise defined, all technical and scientific terms used herein have the same meaning as commonly understood by one of ordinary skill in the art to which this disclosure belongs. The terminology used in the description herein is for describing particular embodiments only and is not intended to be limiting of the disclosure. All publications, patent applications, patents, figures and other references mentioned herein are expressly incorporated by reference in their entirety.

### I. Definitions

As used herein, the following terms may have meanings ascribed to them below, unless specified otherwise. However, it should be understood that other meanings that are known or understood by those having ordinary skill in the art are also possible, and within the scope of the present disclosure. All publications, patent applications, patents, and other references mentioned herein are incorporated by reference in their entirety. In the case of conflict, the present specification, including definitions, will control. In addition, the materials, methods, and examples are illustrative only and not intended to be limiting.

Where a range of values is provided, it is understood that each intervening value, to the tenth of the unit of the lower limit unless the context clearly dictates otherwise, between the upper and lower limit of that range and any other stated or intervening value in that stated range is encompassed within the description. Ranges from any lower limit to any upper limit are contemplated. The upper and lower limits of these smaller ranges which may independently be included in the smaller ranges is also encompassed within the description, subject to any specifically excluded limit in the stated range. Where the stated range includes one or both of the limits, ranges excluding either both of those included limits are also included in the description.

It must be noted that as used herein and in the appended claims, the singular forms “a”, “an”, and “the” include plural references unless the context clearly dictates otherwise.

All numerical values within the detailed description and the claims herein are modified by “about”, “substantially” or “approximately” the indicated value, and take into account experimental error and variations that would be expected by a person having ordinary skill in the art. For example, the terms “about”, “substantially” or “approximately” may be construed as including a deviation of the term they modify, such as by 1%, 2%, 5%, or 10%, for example, if this deviation does not negate the meaning of the modified term.

Furthermore, the recitation of numerical ranges by endpoints herein includes all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the end result is not significantly changed, such as plus or minus 1%, 2%, 5%, 10%, 10%-15%, 5-10%, or 1%-5%, for example.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of” or, when used in the claims, “consisting of” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

The term “cis-Regulatory Element” or “CRE” as used herein refers to a non-coding genomic element which is involved in the regulation of expression of one or more neighbouring genes. Such elements include but are not limited to transcription factor binding sites, promoters, enhancers, and silencers. A particular CRE present in a genome may exist in one or more “states”. By way of example, a CRE that harbours a transcription factor binding site may be bound or occupied by the transcription factor, or may unoccupied by the transcription factor. In other cases, a CRE may be occupied by a histone, for example a modified histone such as H3K4me1, H3K4me3, H3K27ac, H3K9me3, H3K27me3 or H3K36me3. These “states” may be determined for example by sequence data obtained in assays designed to detect the genomic element of interest i.e. a chromatin profile. Accordingly, it will be understood that a CRE may be described as being for example “detected”/“present”, or “absent” in a sample, depending on whether the genomic sequence corresponding to the CRE is detected (e.g. accessible), or “called”, in the chromatin profile used.

The term “chromatin profile” as used herein includes chromatin accessibility profiles, histone modification chromatin profiles, transcription factor binding profiles, and the like, such as but not limited to profiling methods that provide genomic regions as their output like mutated genomic regions, methylated genomic regions, gene promoters, transcription start sites, enhancers, etc. The chromatin profiles can be provided by (e.g. contained within) one or more input data files that may be provided by a user, obtained for storage systems or from public repositories/databases, optionally signal intensity or BED files, used by CREAM to identify clusters of genomic regions such as COREs and LOCKS. The chromatin profiles can be provided in BED format, which comprise tab separated files with for example, the 1^{st} column comprising the chromosome name, the 2^{nd} column comprising the starting position of the genomic region and the 3^{rd} column comprising the end position of the genomic region.

The term “chromatin accessibility profile” as used herein refers to genomic sequence that reflects open chromatin including but not limited genomic sequence obtained using Assay for Transposase-Accessible Chromatin with high-throughput sequencing (ATAC-seq) and DNase I hypersensitive sites sequencing (DNAse-seq). Other datasets can also be used.

The term “histone modification profile” as used herein refers to genomic sequence that reflects the presence of specific modified histones, or histone marks, such as H3K4me1, H3K4me3, H3K27ac, H3K9me3, H3K27me3 and/or H3K36me3. Other histone modifications include, but are not limited to, H3K4me2, H3K9me1, H3K9me2, H3K27me1, H3K27me2, H3K36me1, H3K36me2, H3K79me1, H3K79me2, H3K79me3, H3K9ac, H3K14ac, H3K18ac, H3K56ac, H3ser10P, H3ser28P. Total histone H3 may also be used. Histone modification profiles may be obtained for example using ChIP-seq. The histone modification profile may also be provided in one or more input data files that may be provided by a user, obtained for storage systems or from public repositories/databases.

The term “COGR signature standard” as used herein refers to one or more genomic regions identified to be or comprise (or to not be or not comprise) a COGR, such as a CORE, that is associated with a known phenotype or parameter such as drug sensitivity, stemness or outcome, for example the genomic region or regions associated with the parameter such as tissue type or cancer prognosis, and the associated parameter The COGR signature standard comprises the CORE (e.g. its chromatin position e.g. chromosome and base pair location) and the associated parameter or phenotype, e.g. tissue type, outcome, cell stemness, drug sensitivity, prognosis and the like. Multiple COGR signature standards can be used.

It should also be understood that, in certain methods described herein that include more than one step or act, the order of the steps or acts of the method is not necessarily limited to the order in which the steps or acts of the method are recited unless the context indicates otherwise.

Although any methods and materials similar or equivalent to those described herein can also be used in the practice or testing of the present disclosure, examples of methods and materials are now described. Features described herein, for example in different sections, can be combined unless according to the text, the features are inconsistent.

### II. Methods

Described herein in some embodiments are methods for identifying clusters of genomic regions (COGRs) such as Clusters of cis-Regulatory Elements (COREs) and large organized chromatin lysine (K) element (LOCKs) and determining a COGR signature such as a CORE signature or a LOCK signature in genomic DNA from a biological sample. COGRs are identified using the CREAM technique as further described herein.

As described herein in detail, the method of determining a COGR signature in a biological sample can comprise:

obtaining a chromatin profile, optionally a chromatin accessibility profile or a histone modification profile, of genomic DNA of the biological sample;

identifying two or more individual cis-regulatory elements (CREs) in the genomic DNA chromatin profile; and

locating one or more Clusters of cis-Regulatory Elements (COREs) to generate a CORE signature or one or more Large Organized Chromatin Lysine element (LOCKs) to generate a LOCK signature, comprising the steps of:
i) grouping different numbers of neighboring individual CREs throughout the genome and categorizing the groups according to order (O) which is the number of neighboring individual CREs in the groups;
ii) identifying a maximum window size (MWS) by estimating a distribution of window sizes for each order O based on a maximum distance between the individual CREs in all groups of that order O within the chromatin profile and calculating the MWS according to the following:
   a) MWS=Q1(log(WS))−1.5IQ(log(WS)), where Q1(log(WS)) and IQ(log(WS)) are first quartile and interquartile distributions of window sizes of that order O, respectively; or
   b) MWS=Q1(log(WS_{normalized}))−1.5*IQ(log(WS_{normalized})) where

$$W {S}_{normalized} = max ( \frac{\begin{matrix}distance between two consecutive \\ elements in the group of CREs\end{matrix}}{\begin{matrix}average size of the two consecutive \\ elements in the group of CREs\end{matrix}} ) ;$$

iii) identifying a maximum O (Omax), defined as a value of given order O at which an increase in the given order O does not result in an increase in the MWS for the given order O;
iv) identifying potential COREs or potential LOCKs by calling groups of CREs of a particular order O with a window size less than the MWS for the particular order O as the potential COREs or the potential LOCKs, for each order O from Omax to O=2; and
v) for each order O from O=2 to Omax, calculating the change in (MWS-median(WS))/median(WS), where WS is a distribution of maximum distance between individual CREs within the potential COREs, or the potential LOCKs of that order and filtering out lower order potential CORES or potential LOCKs up to a point where (MWS-median(WS))/median(WS) decreases with increasing order O, and any remaining potential COREs or potential LOCKs, are identified as actual COREs or actual LOCKs and included in the CORE signature or the LOCK signature.

Where the chromatin profile is a chromatin accessibility profile such ATAC-seq or DNAse-seq as well as other chromatin accessibility profiles, the methods can be used for determining a CORE signature in the biological sample. As shown herein, the inventors have demonstrated that CORE signatures can be used for identifying biomarkers of various phenotypes, including drug sensitivity, associated with prognosis, stemness and tissue of origin as well as for identifying the stemness of mixed populations such as tumor cells to assess whether a tumor is enriched for stem cells or not.

Where the chromatin profile comprises a histone modification profile such as H3K4me3, H4K4me1, H3K27ac, H3K27me3 and H3K9me3 as well as other histone modification profiles, the methods herein can also be used to identify Large Organized Chromatin Lysine (K) domains (LOCKs). As shown herein, LOCKs from a collection of histone modifications, or marks, which discriminate primitive from differentiated cell types. Specifically, LOCKs of active histone modifications, including H3K4me1, H3K4me3 and H3K27ac cover a higher fraction of the genome in primitive compared to differentiated cell types. In contrast, LOCKs of repressive marks, including H3K9me3, H3K27me3 and H3K36me3 do not discriminate primitive from differentiated cell types. Active LOCKs in differentiated cells lie proximal to highly expressed genes while active LOCKs in primitive cells tend to be bivalent, harbouring both active and H3K27me3 signals. Genes proximal to bivalent LOCKs are minimally expressed in primitive cells, relating to development and differentiation pathways. Furthermore, bivalent LOCKs populate TAD boundaries and are preferentially bound by regulators of chromatin interactions, including CTCF, RAD21 and ZNF143. Accordingly, LOCKs discriminate primitive from differentiated cell populations, as they relate to transcription regulating events.

As described herein, LOCKs from six post-translational modifications to histone tails (H3K4me1, H3K4me3, H3K27ac, H3K9me3, H3K27me3 and H3K36me3 assessed by ChIP-seq) behave differently across 13 primitive, 9 ES-derived and 77 differentiated phenotypes. As shown herein, primitive cells harbor bivalent LOCKs found at TAD boundaries and enriched for regulators of chromatin interactions. These bivalent LOCKs transit to a repressed state by acquiring H3K9me3 signal while losing H3K27me3 and H3K4me1 in differentiated cells.

A LOCK signature can be similarly identified, when the chromatin profile is for example a histone modification profile and the method further comprises vi) repeating steps i) to v) until the parameter defined by the equation

$$Relative sum - \frac{sum of coverage of LOCKs by individual elements}{sum of total genome coverage of LOCKs}$$

starts oscillations of >5%.

Further details on the method of identifying COGRs is provided herein.

The COGR signatures can be used in a variety of methods as demonstrated in the Examples including, for example, for one or more of identifying biomarkers such as diagnostic or prognostic biomarkers, prognosing a patient outcome, identifying a phenotype such as stemness, genomic or gene analysis of identified COGRs, drug target identification and determining cell or tissue type, for example tissue of origin of a biopsy.

For example, after identifying a COGR signature, the method can comprise genomic or gene analysis of the identified COGRs. This may involve querying genomic structure or genes proximal to the COGRs, for example within 0-25 kb up or downstream from an end of the identified COGR. This method may be performed by a gene or genomic analysis module 129 that can be used on its own or with as part of another software modules that perform other methods such as drug discovery. In some embodiments, a gene is considered associated with a CRE or a CORE if the CRE or CORE is found within a ∓25 kb, ∓20 kb, ∓15 kb, ∓10 kb, ∓5 kb, ∓2.5 kb, ∓1 kb, or any number in between ∓25 kb to ∓1 kb window from the TSS of the gene. This can be used to identify relevant active biological pathways associated with the phenotype.

The methods can be performed for a plurality of biological samples, wherein each of the biological samples has an associated phenotype for example a cancer type. CORE signatures which identify the phenotype, for example as determined in training sets, can be used as a CORE signature standard for the phenotype. The methods can also be performed for a plurality of biological samples wherein each of the biological samples has an associated phenotype of a plurality of phenotypes. CORE signatures which distinguish a phenotype from the plurality of phenotypes, for example as determined in training sets, can be used to provide a plurality of COGR signature standards each associated with a different phenotype for identifying if a test biological sample is most similar to one or another of the plurality of phenotypes.

Accordingly, also described herein are methods for identifying biomarkers associated with a phenotype, optionally drug sensitivity, tissue or cell type, differentiation or stemness, enrichment for stem cells or prognostic biomarkers.

In an embodiment, the method for identifying a biomarker associated with a selected phenotype comprises:

determining a COGR signature as described herein, particularly a CORE signature, for a plurality of biological samples, each of the biological samples having an associated or determined phenotype for one of a plurality of phenotypes;

assessing each COGR of each COGR signature to build a univariate predictive model of outcome;

ranking each COGR according a phenotype prediction,

optionally generating a multivariate prediction model using two or more ranked COGRs,

wherein one or more of the COGRs of the COGR signature with phenotype prediction above a selected threshold is the biomarker associated with the selected phenotype and optionally is included in a COGR signature standard.

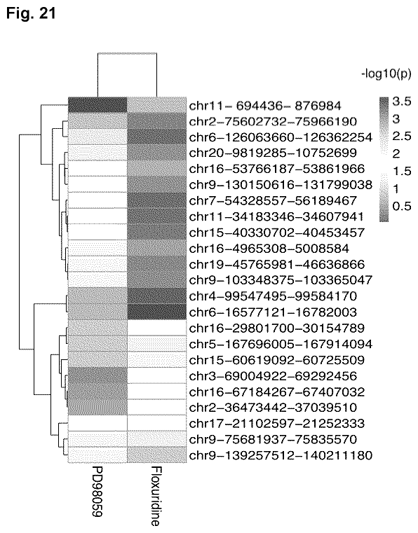

For example, the plurality of phenotypes can be binary, for example the phenotype can be drug sensitivity (responder) or the phenotype can be drug insensitivity (non-responder) and greatest similarity is determined by determining if a CORE or COREs is/are present or absent. The data may be obtained or determined. For example, different biological samples (e.g. different cell lines or primary cells) can be treated with a drug to ascertain whether they are sensitive to a drug or insensitive to a drug, and the ATAC-seq data obtained. If such genomic and phenotype data is available it can be obtained as the input file. As described in Example 15 and shown in FIG. 21 and Tables 2 and 3, top biomarkers were identified using COREs as biomarkers of response to PD98059 and Floxuridine tested on 16 breast cancer cell lines in the GRAY dataset with available ATAC-seq profiles. Biomarkers of response to these drugs in the 16 breast cancer cell lines (FDR<0.05) included those listed in Table 2 such as chr11:694436-876984 and those listed in Table 3 such as chr4:99547495-99584170 for PD98059 and Floxuridine, respectively.

For sets of samples with available chromatin accessibility profiles (ATAC-seq, DNaseI, or any other chromatin accessibility profile) and for example drug sensitivity data, COREs of the samples are first identified using the methods described herein. A catalogue of COREs of all samples can be obtained. Each CORE of each sample can be mapped to the catalogue of COREs of all the samples. The mapping, involves identifying if a CORE of a sample overlaps with a CORE in the catalogue providing a binary feature matrix (0 or 1 features) in which rows are samples and COREs in the catalogue are features. Various approaches can be used for biomarker discovery. For example Sparse learning methods like elasticnet and Lasso can be used. In such approaches, the binary features matrix is used as the feature matrix (or data frame) and sensitivity of samples to the given drug is used as the output variable. At the end, the resulting feature set and their coefficients can be used as multivariate biomarker of drug response. Alternatively, it is possible to use a univariate statistical inference. For example, statistical measures like the Matthews correlation coefficient (MCC) can be used to compare drug sensitivity of samples that have a specific feature (e.g. CORE) in the catalogue versus the ones where the CORE is absent. Then the COREs with significant difference in the drug sensitivities of the samples are identified as significant or top COREs.

In some embodiments, the phenotype can be enrichment for stem cells in a tumor sample.

For example, the methods described herein can be used to identify enrichment of cancer stem cells within a bulk tumor sample. As described in the Examples, a collection of stem cell enriched and non-stem cell enriched ATAC-seq profiles across multiple tumor types including Leukaemia, Glioblastoma Multiforme (GBM), Pancreatic Adenocarcinoma and Colon Adenocarcinoma were assessed. Clusters of cis-regulatory elements (COREs) for these samples identified using methods described herein were identified. A test sample of a known tumor type (e.g. specified by user) can be assessed to identify its associated COREs, the identified COREs are compared with COREs identified for the collection of stem cell enriched and non-stem cell enriched samples. The difference between these two similarities, similarity to stem cell enriched samples and non-stem cell enriched samples, can be reported as the stem-score of the sample.

In one embodiment, the method comprises

determining a COGR signature of a biological sample of known tumor type, according to a method described herein;

assessing a similarity of the COGR signature of the biological sample with at least one COGR signature standard of a tumor sample known to be stem cell enriched and at least one COGR signature standard of a tumor sample known not to be stem cell enriched;

determining any difference between the similarity to the at least one COGR signature standard known to be (e.g. associated with being) stem cell enriched and the at least one COGR signature standard known not to be (e.g. associated with lacking being) stem cell enriched; and

assigning a score indicative of the stemness of the biological sample.

In one embodiment, the tumor type is leukemia. Other tumor types can also be used.

The methods are particularly useful for comparing stem cell rich and stem cell deficient types of tumor cells. The stemness score indicative of increased stemcell identity may be more aggressive, warranting for example a more aggressive treatment. The stemness score for a test biological sample for example a cancer test biological sample, can be assessed for example by assessing the similarity of the sample COGR signature to cancer stem cell and cancer non-stem cells. For example, the average of its Jaccard similarities to cancer non-stem cell samples (in a training dataset) can be deducted from an average of the its Jaccard similarities to cancer stem cell samples resulting in the stemness score.

In some embodiments, the stemness score will be bounded between −1 and 1, where a score of 1 identifies stem cells. A score closer to 1 than −1 thereby indicates that the test biological sample is more similar to the stem cell samples and has higher stemness capacities.

Also demonstrated herein, are methods for identifying drug target for a phenotype of interest using its COGR signature. This can be done for example, where the CORE signature of a biological sample having a phenotype of interest is compared to a CORE signature standard or standards for example determined from a plurality of samples or tissue that lack the phenotype of interest and a plurality of samples or tissues that comprise the phenotype of interest.

Accordingly, in an embodiment, the method for identifying a drug target comprises

determining a COGR signature as described herein, optionally a CORE signature, for a plurality of biological samples, each of the biological samples having an associated or determined phenotype for one of a plurality of phenotypes;

assessing each COGR of each COGR signature to build a predictive model of each phenotype;

ranking each COGR according to phenotype prediction to identify top COGRs,

identifying individual CREs within one or more of the top COGRs that are specific for a selected phenotype of the plurality of phenotypes,

determining a COGR signature as described herein for cells genetically deleted of one or more of the individual CREs determined to be present in cells of the selected phenotype;

determining if the genetically deleted cells have an altered or non-selected phenotype; and

identifying one or more of the ranked COGRs as potential drug targets if the COGR signature of the genetically deleted cells is more similar to the non-selected phenotype.

The method can also comprise genetically deleting or otherwise modifying one or more of the individual CREs in a population of cells corresponding to the selected phenotype and assessing if the genetic deletion or modification changes the cell population so that it is more similar to the non-selected phenotype than the selected phenotype. This can be used to validate the COGR signature identified.

The identified COGR signature or individual COGRs thereof can then be used to identify drug targets. The biological samples can for example be different samples identified to have a stem cell phenotype or a non-stem cell phenotype. For example, as shown in the Examples, a leukemia stem cell positive samples and negative samples were compared and the potential target (chr9: 2014811-2032652) was identified to be present predominantly in LSC+ samples. The inventors then identified individual CREs in the top CORE that was specific to LSC+ and found that the CORE signature of the genetically modified cells was more similar to LSC− cells. The potential drug target CORE can be used to identify genes that are regulated by the CORE and/or affected by genetically deleting the CREs, to identify druggable targets for the LSC+ phenotype. The deletion can be in a cell line from which the CORE or COREs was/were identified or in a similar cell line, such as a patient derived cell line that has a similar COGR signature, and minimally comprise the COGR to be deleted or otherwise modified.

As further described herein, the step of assessing each COGR of each COGR signature to build a predictive model of each phenotype can involve creating a COGRs catalogue feature matrix. The feature matrix is use to provide a predictive model for example by training an elasticnet model. Various parameters can be used to increase the accuracy, including for example implementing a 5 fold cross validation and repeating it 100 to decrease overfitting caused by cross validation. Such a model can produce a coefficient for each COGR in the catalogue across the 100 time 5 fold cross validation. The coefficient is a predictive indicator of the likelihood of the COGR in the particular phenotype.

The COGRs catalogue feature matrix may be used in various applications and can be a binary matrix with rows corresponding to biological sample and/or phenotype (e.g. patient tumor samples, cancer cell lines etc.) and the columns comprising the features, which are the COGRs identified. Each element of the matrix indicates if a particular COGR is present in the biological sample and/or phenotype.

In another example, the phenotype can be cell or tissue of origin. For example, the methods described herein are able to classify a sample according to its tissue of origin.

In an embodiment, the method comprises determining a plurality of COGR signature standards for known tissues or corresponding cell lines and then comparing a CORE signature of a test biological sample, to the plurality of COGR signature standards, and identifying a COGR signature standard of the plurality most similar to the CORE signature to identify the likely tissue of origin.

A COGR can be assigned to a cell, tissue or differentiation status if for example the COGR, optionally a LOCK, is identified if at least 40%, at least 45% or at least 50% of the biological samples assayed when identifying a COGR signature standard.

In the Examples, a LOCK was assigned to each tissue type if it existed in more than 50% of samples from that tissue type.

As shown in the Examples, identification of cell or tissue of origin is robust when multiple COGR signature standards for the same or similar tissue types are used. Accordingly, the COGR of the biological sample is optionally compared to a set of COGR signature standards, the set comprising signature standards for at least 3, at least 4 or at least 5 tissue samples or corresponding cell types.

In another example, the phenotype can be disease outcome, for example good disease outcome or poor disease outcome.

In one embodiment, the method is for identifying a prognostic biomarker the method comprising:

determining a COGR signature as described herein, particularly a CORE signature, for a plurality of tumor samples of a tumor type, each of the tumor samples having associated outcome data;

assessing each COGR of each COGR signature to build a univariate predictive model of outcome;

ranking each COGR according a risk prediction,

optionally generating a multivariate prediction model using two or more ranked COGRs,

wherein one or more of the identified COGRs with risk prediction above a selected threshold is the prognostic biomarker.

As shown in the Examples, the COREs identified are used to predict survival of the patients in univariate models. For example, each CORE is used as binary feature (e.g. exist in a sample or not) and then its existence is examined for its ability to be predictive of survival in cancer patients. Tops COREs for example with a selected minimum false discovery rate (FDR) (significance after multiple hypothesis correction) and dindex>1 (where dindex is a measure of hazard) can be considered for combination.

The outcome can also be related to treatment outcome, e.g. good prognosis when treated with a particular drug, poor prognosis when not treated.

Top COREs can be the COREs where their FDR is equal to the minimum FDR of all COREs, for example if more than 1 CORE has the minimum value.

In some embodiments, COREs that exist in less than 5 samples, for example where a large number of samples are assessed, is further filtered. The remaining COREs can be combined for example as described in the Examples.

Various biomarker methods for example, include selection of top COGRs. Top COGRs such as COREs can also be selected using other measures that identify the significance or effect size of association between existence (in case of binary features like COREs) or value of that feature and the output value (like drug sensitivity in case of biomarker discovery). FDR which is the corrected p-value for multiple hypothesis is one approach. Fold change is another example.

For example, in methods involving assessing drug sensitivity, for each CORE, average drug sensitivity of the samples with that CORE can be divided to the average drug sensitivity of the samples without that CORE. Then the COREs can be sorted based on the obtained fold changes and COREs with top fold changes can be selected.

As indicated herein, dindex can be used for identifying top COREs. Hazard ratio can also be used.

As further shown in the Examples, the inventors identified various prognostic biomarkers. The method of identifying a prognostic marker can include one or more of the steps indicated in the Examples. The prognostic markers can be used to prognose a patient's outcome.

The phenotypic biomarker or biomarkers, optionally the prognostic COREs can be used as a COGR signature standard for assessing test biological samples such as patient samples. Biological samples that are assessed for similarity to one or more COGR signature standards can be referred to as test biological samples. COGR signature standards can be determined by clustering to identify if a COGR or COGRs is or are associated with a phenotype, optionally a selected phenotype. Clustering methods that can be used include hierarchical clustering using ward.D2 as the method for identifying distance between groups of samples.

Accordingly, also described herein are methods for determining the prognosis of a patient diagnosed with a cancer on the basis of a COGR signature optionally a CORE signature, of a biological sample obtained from the patient.

In one embodiment, the method for determining a prognosis of a patient comprises:

determining a COGR signature of a biological sample previously acquired from the patient according to a method described herein;

comparing the COGR signature of the biological sample with one or more COGR signature standards, each COGR signature standard associated with an outcome;

providing the patient with a prognosis according to the COGR signature standard most similar to the COGR signature.

In one embodiment, the COGR signature is a CORE signature. Instead of a CORE signature, a LOCK signature can also be determined.

Different prognostic measures may be given. A prognosis may, for example and without limitation, be given in terms of overall survival, net survival, observed survival, relative survival, disease-free survival, progression-free survival, or other measures such as median survival and stated as poor, very poor or good survival rates. Survival rates may be given in terms of years, such as for example a one year survival rate, a two year survival rate, a three year survival rate, a four year survival rate, a five year survival rate, or a ten year survival rate. Poor survival rate may include an expected survival rate of about 0 within 4 years and very poor may include an expected survival rate of about 0 within 2 years. A good survival rate can include an expected survival rate above 40% after 4 years.

As demonstrated herein, the presence of a COGR such as a CORE at one or more specific chromosomal locations in tumor samples taken from patients diagnosed with a cancer such as lung cancer, colon cancer, or liver cancer is indicative of a poor prognosis. The identified COREs correspond to and/or are included in the CORE signature standard for prognosing the patient. Also demonstrated herein, the presence of a CORE at more than two specific chromosomal locations in tumor samples taken from patients diagnosed with a cancer such as kidney renal papillary cell carcinoma is indicative of a good prognosis. Further demonstrated herein, the presence of one or no COREs at specific chromosomal locations in tumor samples taken from patients diagnosed with a cancer such as stomach adenocarcinoma is indicative of poor prognosis. Accordingly, in some embodiments, the patient has been diagnosed with lung adenocarcinoma, colon adenocarcinoma, kidney renal papillary cell carcinoma, stomach adenocarcinoma, lung squamous cell carcinoma, or liver hepatocellular carcinoma and the prognosis is determined on the basis of the presence or absence of a COGR, optionally a CORE at one or more specific chromosomal locations.

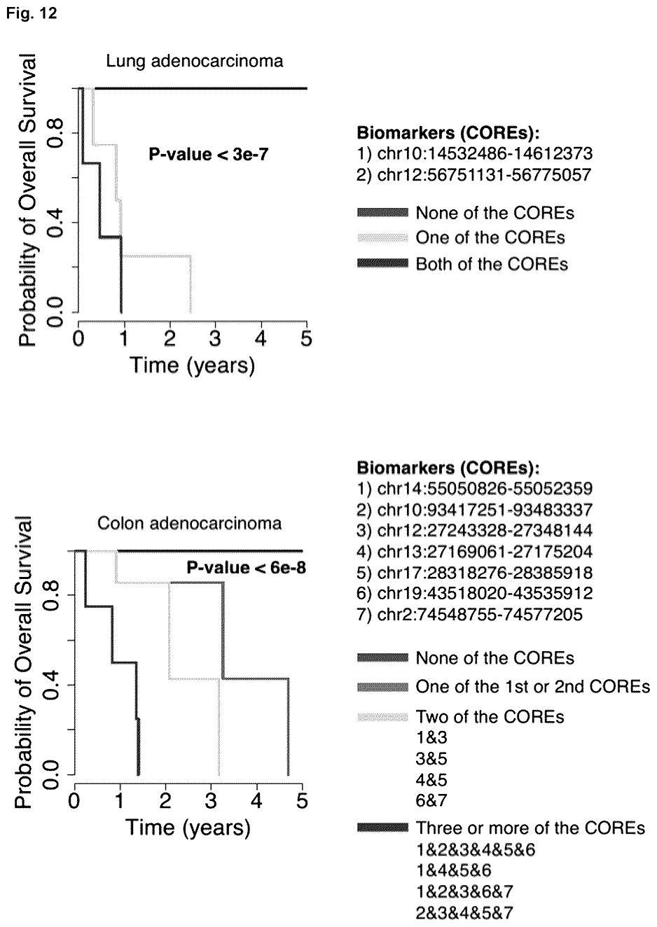

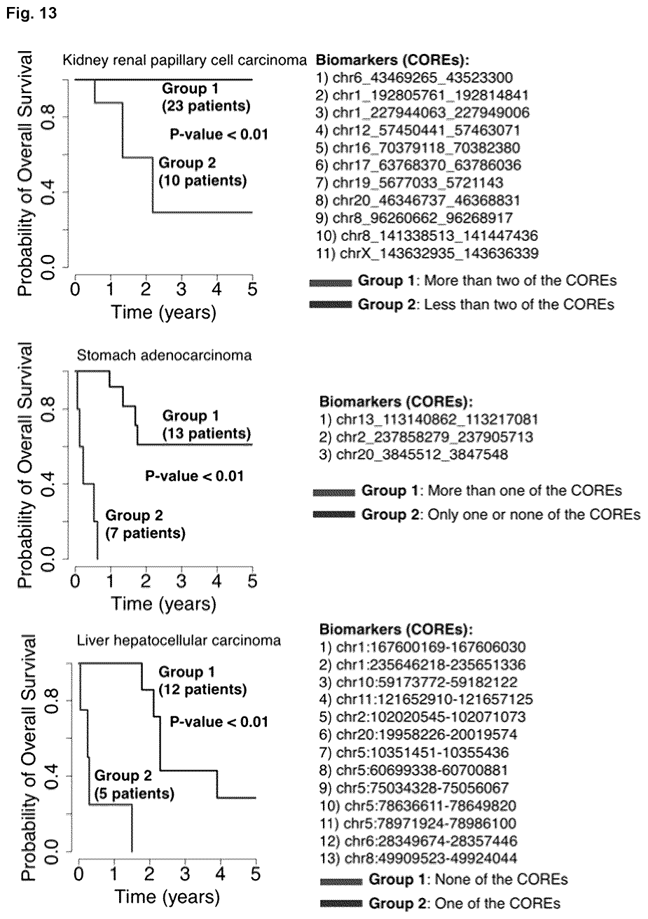

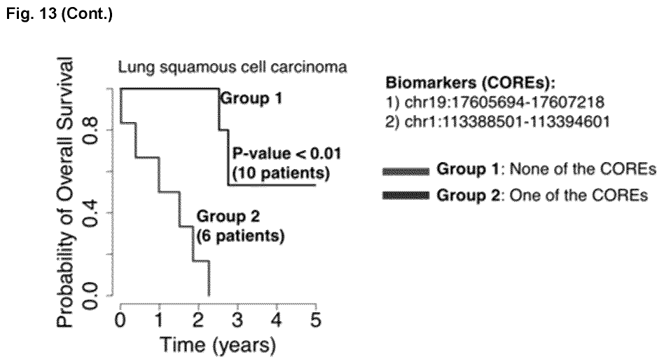

Any of the identified CORE biomarkers described herein can be used in the prognostic methods, including for example COREs identified in FIGS. 12 and 13. The signature standard can comprise one or more of the COREs identified therein.

In one embodiment, CORE signature standard for lung adenocarcinoma comprises 2 chromosomal locations. In another embodiment, the CORE signature standard for colon adenocarcinoma comprises 7 chromosomal locations as described herein.

In one embodiment, the patient has been diagnosed with lung adenocarcinoma, and the CORE signature comprises chr10:14532486-14612373 and/or chr12:56751131-56775057 and the prognosis of the patient is determined to be poor, optionally a three year survival rate of zero when a CORE is detected at chr10:14532486-14612373 or chr12:56751131-56775057 and good when said CORE is not detected in the CORE signature.

In another embodiment, the prognosis is determined to be very poor, optionally a one year survival rate of zero when a CORE is detected at both chr10:14532486-14612373 and chr12:56751131-56775057 and good when said COREs are not both detected in the CORE signature.

In yet another embodiment, the patient has been diagnosed with colon adenocarcinoma and the CORE signature comprises one or more of the following chromosomal locations:

b) chr14:55050826-55052359;

c) chr10:93417251-93483337;

d) chr12:27243328-27348144;

e) chr13:27169061-27175204;

chr17:28318276-28385918;

g) chr19:43518020-43535912; or

h) chr2:74548755-74577205.

In one embodiment, the prognosis is determined to be poor, optionally a five year survival rate of zero when a CORE is identified at least one of a) or b) and good when said CORE is not detected in the CORE signature.

In another embodiment, the prognosis is determined to be poor, optionally a four year survival rate of zero when a CORE is identified at two or more of a) through g), optionally, a CORE is identified at: a) and c); c) and e); d) and e); or f) and g) and good when said COREs are not detected in the CORE signature.

In one embodiment, the prognosis is determined to be very poor, optionally a two year survival rate of zero when a CORE is identified at three or more of a) through g), optionally a CORE is identified at: a), b), c), d), e) and f); a), d), e) and f); a), b), c), f) and g); or b), c), d), e) and g) and good when said COREs are not detected in the CORE signature.

In another embodiment, the patient has been diagnosed with kidney renal papillary cell carcinoma, and the CORE signature standard two or more comprises chromosomal locations:

a) chr6_43469265_43523300;

b) chr1_192805761_192814841;

c) chr1_227944063_227949006;

d) chr12_57450441_57463071;

e) chr16_70379118_70382380;

f) chr17_63768370_63786036;

g) chr19_5677033_5721143;

h) chr20_46346737_46368831;

i) chr8_96260662_96268917;

j) chr8_141338513_141447436; and/or

k) chrX_143632935_143636339

wherein the prognosis of the patient is determined to be good if a CORE is identified at more than two of the chromosomal locations.

In an embodiment, the patient has been diagnosed with stomach adenocarcinoma, and the CORE signature standard comprises one or more of the chromosomal locations:

l) chr13_113140862_113217081;

m) chr2_237858279_237905713; and/or

n) chr20_3845512_3847548

wherein the prognosis of the patient is determined to be very poor, optionally a one year survival rate of zero, when a CORE is identified at only one or none of the chromosomal locations in the CORE signature.

In an embodiment, the patient has been diagnosed with liver hepatocellular carcinoma, and the CORE signature standard comprises one or more of the following chromosomal locations:

a) chr1:167600169-167606030;

b) chr1:235646218-235651336;

c) chr10:59173772-59182122;

d) chr11:121652910-121657125;

e) chr2:102020545-102071073;

f) chr20:19958226-20019574;

g) chr5:10351451-10355436;

h) chr5:60699338-60700881;

i) chr5:75034328-75056067;

j) chr5:78636611-78649820;

k) chr5:78971924-78986100;

l) chr6:28349674-28357446; and

m) chr8:49909523-49924044

wherein the prognosis is determined to be very poor, optionally a two year survival rate of zero, when a CORE is identified at one or more of the chromosomal locations in the CORE signature.

In another embodiment, he patient has been diagnosed with lung squamous cell carcinoma, and the CORE signature standard comprises at one or more of the following chromosomal locations:

a) chr19:17605694-17607218; and

b) chr1:113388501-113394601

wherein prognosis of the patient is determined to be poor, optionally a three year survival rate of zero, when a CORE is identified at one or more of the chromosomal locations in the CORE signature

As mentioned herein, the methods can be used to identify drug sensitivity biomarkers where chromatin profiles are available for biological samples with associated drug response or obtained as described herein and a patient can be assessed for whether the patient is likely to respond or not respond to a particular drug or combination of drugs.

Similar methods can be used when assessing whether a subject is more likely to be respond to a particular treatment. In such cases the COGR signature standard is associated with a response and optionally outcome.

Biomarker markers with significant association with PD98059 and floxuridine are shown in Tables 2 and 3 respectively. For example, in the case of breast cancer, chr11:694436-876984 and/or chr4:99547495-99584170 can be used to identify a patient's likelihood to respond to PD98059 or Floxuridine, respectively.

Accordingly, an aspect includes a method of selecting a treatment for a patient with cancer, the method comprising:
determining a COGR signature of a biological sample previously acquired from the patient as described herein;
comparing the COGR signature to one or more COGR signature standards having an associated drug sensitivity, optionally identified using a biomarker discovery method described herein;
selecting a treatment according to the associated drug sensitivity of the COGR signature standard with the greatest similarity.

The COGR signature can be a CORE signature and can the COGR signature standards can include a CORE identified in Table 2 or 3.

In an embodiment, is provided a method of selecting a treatment for a patient with cancer, the method comprising:
determining a CORE signature of a biological sample previously acquired from the patient as described herein, wherein the biological sample is a breast cancer sample, comparing the CORE signature to one or more CORE signature standards having an associated drug sensitivity, the CORE signature standard for PD98059 drug sensitivity comprising a biomarker in Table 2 and the CORE signature standard for floxuridine drug sensitivity comprising a biomarker in Table 3, and
selecting a treatment according to the associated drug sensitivity of the CORE signature standard with the greatest similarity.

For example, if a patient is identified to have a CORE signature comprising one or more of chr11-694436-876984, chr11-34183346-34607941, chr15-40330702-40453457, chr16-4965308-5008584, chr19-45765981-46636866, chr9-130150616-131799038 and/or chr9-139257512-140211180, the patient is more likely to respond to a treatment comprising MEK inhibitor such as PD98059 (e.g. more likely than if the one or more COREs is/are absent). If a patient is identified to have a CORE signature comprising one or more of chr16-53766187-53861966, chr17-21102597-21252333, chr2-75602732-75966190, chr20-9819285-10752699, chr6-126063660-126362254, chr7-54328557-56189467, chr9-75681937-75835570 and/or chr9-103348375-103365047, the patient is less likely to respond to a treatment comprising MEK inhibitor such as PD98059 (e.g. less likely than if the one or more COREs is/are absent).

In an embodiment, if a patient is identified as having a CORE signature comprising one or more of chr16-29801700-30154789 and/or chr16-67184267-67407032 is more likely to respond to a treatment comprising a pyrimidine analogue such as floxuridine (e.g. more likely than if the one or more COREs is/are absent). In another embodiment, if a patient is identified as having a CORE signature comprising one or more of chr15-60619092-60725509, chr17-21102597-21252333, chr2-36473442-37039510, chr3-69004922-69292456, chr4-99547495-99584170, chr5-167696005-167914094 and/or chr6-16577121-16782003 the patient is less likely to respond to a treatment comprising a pyrimidine analogue such as floxuridine (e.g. less likely than if the one or more COREs is/are absent).

In an embodiment, the patient has breast cancer.

Also provided are methods of monitoring disease progression. For example, a biological sample of a patient such as a tumor biopsy can be assessed to determine its COGR signature according to a method described herein at a first time point and repeated at a second timepoint, optionally after the patient has been treated, optionally administered one or more doses of a chemotherapy. The COGRs signatures are compared. Changes in the COGRs including those herein associated with outcome can be indicative of whether the patient is responding to treatment.

One or more the prognostic or drug sensitivity biomarkers described herein can be used.

A further aspect is a method of treating a patient the method comprising determining a COGR, optionally CORE signature, of a biological sample previously acquired from the patient according to a method described herein; comparing the CORE signature to one or more CORE signature standards having an associated drug sensitivity, and administering a treatment according to the associated drug sensitivity of the COGR signature standard with the greatest similarity to the COGR signature of the biological sample.

In an embodiment, the method comprises treating a patient comprising a CORE signature, optionally as identified herein, with a suitable treatment.

For example, if a patient is identified to have a CORE signature comprising one or more of chr11-694436-876984, chr11-34183346-34607941, chr15-40330702-40453457, chr16-4965308-5008584, chr19-45765981-46636866, chr9-130150616-131799038 and/or chr9-139257512-140211180, the patient is more likely to respond to a treatment comprising a MEK inhibitor such as PD98059 and the patient is administered a treatment comprising a MEK inhibitor such as PD98059. If a patient is identified to have a CORE signature comprising one or more of chr16-53766187-53861966, chr17-21102597-21252333, chr2-75602732-75966190, chr20-9819285-10752699, chr6-126063660-126362254, chr7-54328557-56189467, chr9-75681937-75835570 and/or chr9-103348375-103365047, the patient is less likely to respond to a treatment comprising MEK inhibitor such as PD98059 and the patient is administered a treatment lacking a MEK inhibitor such as PD98059.

In an embodiment, if a patient is identified as having a CORE signature comprising one or more of chr16-29801700-30154789 and/or chr16-67184267-67407032 is more likely to respond to a treatment comprising a pyrimidine analogue such as floxuridine and the patient is administered a treatment comprising a pyrimidine analogue such as floxuridine. In another embodiment, if a patient is identified as having a CORE signature comprising one or more of chr15-60619092-60725509, chr17-21102597-21252333, chr2-36473442-37039510, chr3-69004922-69292456, chr4-99547495-99584170, chr5-167696005-167914094 and/or chr6-16577121-16782003 the patient is less likely to respond to a treatment comprising a pyrimidine analogue such as floxuridine and is administered a treatment lacking a pyrimidine analogue such as floxuridine.

In an embodiment, the patient has breast cancer.

Also provided are uses of the methods described herein for example for preparing a treatment plan (e.g. whether the patient should be treated or monitored, or they type of treatment, e.g. radiation, chemotherapy or aggressiveness of treatment) identifying information that can aid in a prognosis, or for prognosing the patient. For example, the COGR signature standards can be related to drug sensitivity, with each signature standard indicative of sensitivity or insensitivity to a drug treatment. A patient biological sample can be subjected to COGR analysis to obtain a COGR signature, and the signature can be compared to the one or more COGR signature standards associated with drug sensitivity, wherein the subject is treated or selected to be treated with a drug that the patient's COGR signature indicates the patient is sensitive to or is likely to respond.

Also provided are uses of the methods described herein.

As explained herein, a CORE signature can be obtained when an input file includes data for a chromatin accessibility profile, for example ATAC-seq or DNAse-seq and a LOCK signature can be obtained when the input file includes data for a histone modification profile. The methods identify COGRs that have a start and end position in a chromosome. The chromosome start and end positions disclosed herein refer to genomic or chromosome positions. Chromosome positions can be specific to the genome build. For example, the chromosome start and end positions for the prognostic biomarkers, are based on hg38 and for the leukemia and drug response sensitivity markers, hg19.

The similarity assessment performed by the methods described herein can be implemented using various techniques. For example, similarity can be assessed by determining if a patient sample has one or more COGR which overlap (indicative the COGR is present) with a COGR associated with an outcome. The COGR is for example identified to be present if the COGR regions overlap, for example the patient COGR can be identified with the same start and stop position as the signature standard COGR, can be comprised within the identified region and/or can overlap the region by for example 1 base pair or more, optionally 20, 30, 40, 50, 60 or more base pairs.

Alternatively, similarity can be assessed by measuring one or more of the Jaccard similarity, the Sorensen-Dice coefficient, cosine similarity, using neural networks, decision-tree induction, Bayesian and case based classification. Any statistical measure that compares binary features can be used.

Jaccard similarity for example is the length of intersect of the COREs (as binary features) between two samples (e.g. how many COREs between two samples overlap each other) over length of union of the COREs of the two samples (e.g. total number of COREs in a catalogue of COREs of two samples).

As indicated in the Examples, similarity between two samples for example two samples in the ENCODE or TCGA datasets can by identified relying on Jaccard index for the commonality of their identified COREs throughout the genome. The Jaccard index can be used as the similarity statistics in a 3-nearest-neighbor classification approach. Performance of the classification can be assessed using leave-one-out cross validation. Matthews correlation coefficient can be used for performance of the classification model (Smirnov et al. 2016). Phenotype of each tissue for example can be considered as a class and the obtained vectors used to calculate MCC using the implemented MCC function in PharmacoGx package in R (Smirnov et al. 2016). In this classification scheme, phenotype of the closest sample to an out of pool sample as its phenotype is considered.

Other methods for assessing similarity between datasets can also be used.

Greatest similarity or the most similar is used herein when comparing a COGR signature to one or more COGR signature standards. The COGR signature standard that is most similar or has the greatest similarity is the COGR signature standard of a plurality of COGR signature standards that is most similar to the COGR signature of the biological sample. It is understood herein that the biological sample can be utilized to identify a COGR signature standard for example when identifying biomarkers or can be a test sample for example when assessing a test sample.

In one embodiment, the COGR signature determined is a CORE signature. In another embodiment, the COGR signature determined is a LOCK signature.

A biological sample obtained from any eukaryotic organism and/or tissue type and/or cell culture may be used in the methods described herein. The biological sample may be obtained for example from a tissue sample, saliva, blood, plasma, sera, stool, urine, semen, sputum, mucous, lymph, synovial fluid, cerebrospinal fluid, ascites, pleural effusion, seroma, pus, skin swab, or mucosal membrane surface. Suitable methods for obtaining tissue samples include tissue biopsy, endobronchial biopsy, transbronchial biopsy, brushing cytology, washing cytology, fine needle aspiration cytology, fluid cytology, or bone biopsy.

The chromatin profile which is the CREAM input data may be obtained from one or more sources such as ENCODE available from the ENCODE Project, or TGCA or obtained by assaying a biological sample. Any suitable method for preparing chromatin profiles such as chromatin accessibility profiles and histone modification profiles may be used in the methods described herein. Suitable methods include, without limitation, MNase-seq, ChIP-seq, DNase-seq, Faire-seq and ATAC-seq. In some embodiments, ATAC-seq is used for the chromatin accessibility profile. In some embodiments, ChIP-seq is used for obtaining the histone modification profile. Optionally ChIP-seq is used for assaying open chromatin associated with modified histones such as H3K4me1, H3K4me3, H3K27ac, H3K9me3, H3K27me3 and/or H3K36me3.

ATAC-seq profiles can be prepared for example by harvesting cells from a cell line or tissue such as a biopsy, lysing the cells, optionally with non-ionic detergent, to create a crude nuclei preparation, resuspending nuclei in a transposition reaction mix to fragment the genomic DNA, and purifying the DNA. The DNA fragments can be amplified and further purified and then sequenced. Methods such as those described in Buenrostro et all Curr Protoc Mol Bio 2015; 109:29.1-21.29.9.

Also described herein are methods for determining the tissue of origin or differentiation state of a biological sample on the basis of the CORE signature. Accordingly, in some embodiments the CORE signature is compared to a CORE signature standard or standards for example from a tissue or tissues of known origin to determine the tissue of origin of the biological sample.

For example, the CORE signature standard or standards can be a dataset or can be derived from a dataset such as the ATAC-Seq of the Cancer Genome Atlas (TCGA) as demonstrated in the Examples. This dataset (when queried) comprised profiles of 400 samples across 23 tumor types. Other databases can also be used and/or optionally combined with said database. The assay type (e.g. ATAC-seq/DNase I hypersensitivity) of the query sample can be the same as that of the reference sample or samples.

Fora new test sample, COREs are identified independent of the previous sample or standard, as CREAM does not require any training sample for CORE identification. Chromosomal location of the identified COREs (i.e. the overlap of two COREs at a particular chromosomal location) is used for similarity analyses. The exact pattern of genomic features within a CORE need not be considered.

In another example, the CORE signature can be a dataset or derived from a dataset such as a complete epigenome from the Roadmap Epigenomics Project as demonstrated in the Examples. This dataset comprises the complete epigenomes (H3K4me1, H3K4me3, H3K27ac, H3K9me3, H3K27me3 and H3K36me3 from ChIP-seq) across 13 primitive cell types, including Embryonic Stem Cells (ESCs) and induced Pluripotent Stem Cells (iPSCs) as well as 9 ES-derived and 77 differentiated cell types from diverse tissue or origin⁶.

In some embodiments, the biological sample is from a human subject. The biological sample can be a sample such as a tumor sample that is processed to obtain a chromatin profile or can refer to the source of a chromatin profile for example as provided in a resource such as ENCODE. In some embodiments, the biological sample is a tissue sample, a cell line or an organism (e.g. microbe). In other embodiments the biological sample is from an animal model, much as a mouse model or other model systems like cell lines. The patient is typically human can also be another mammal.

Further details on the method of identifying COGRs and COGR signatures is now provided.

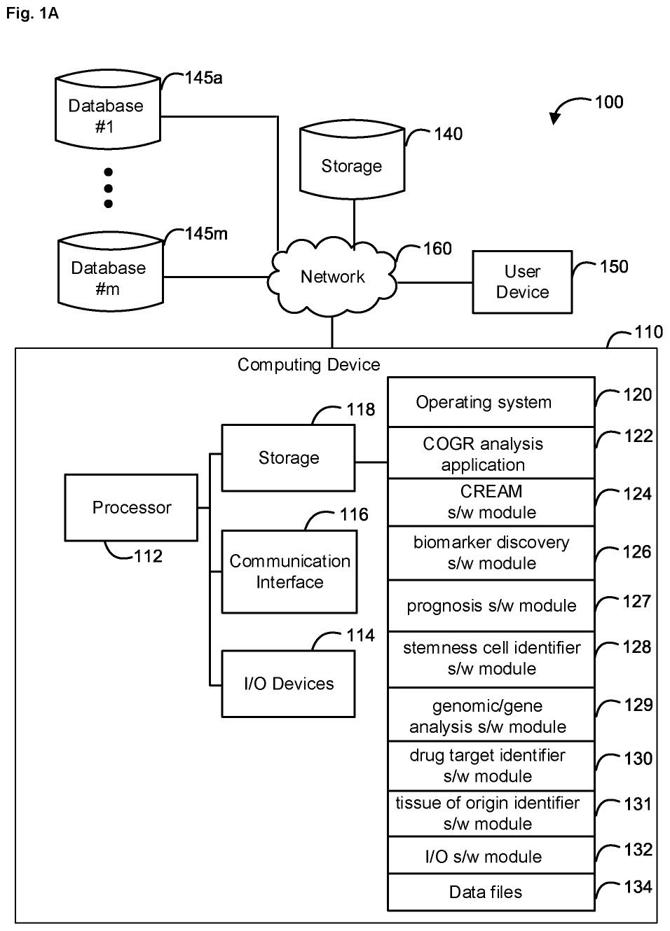

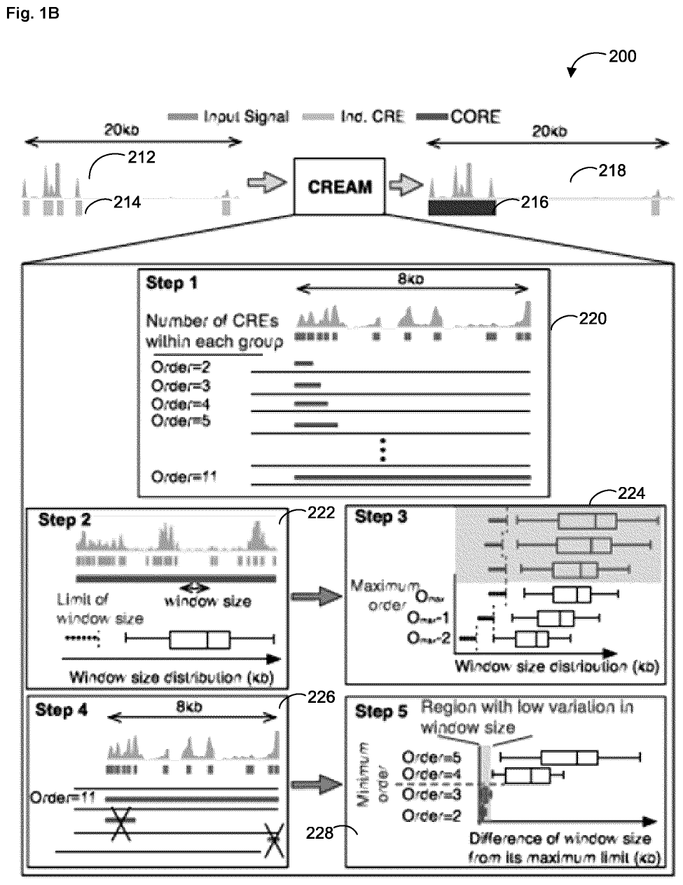

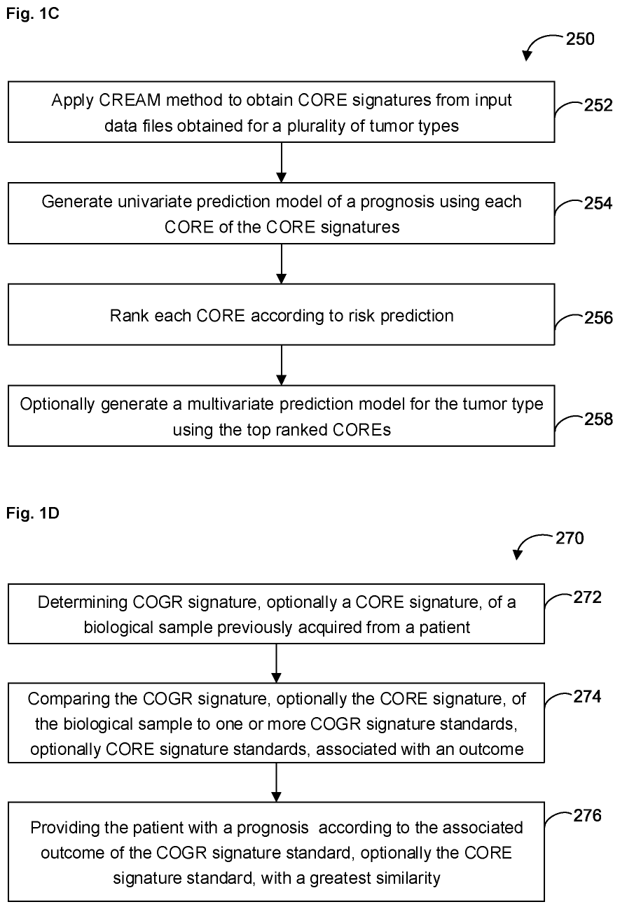

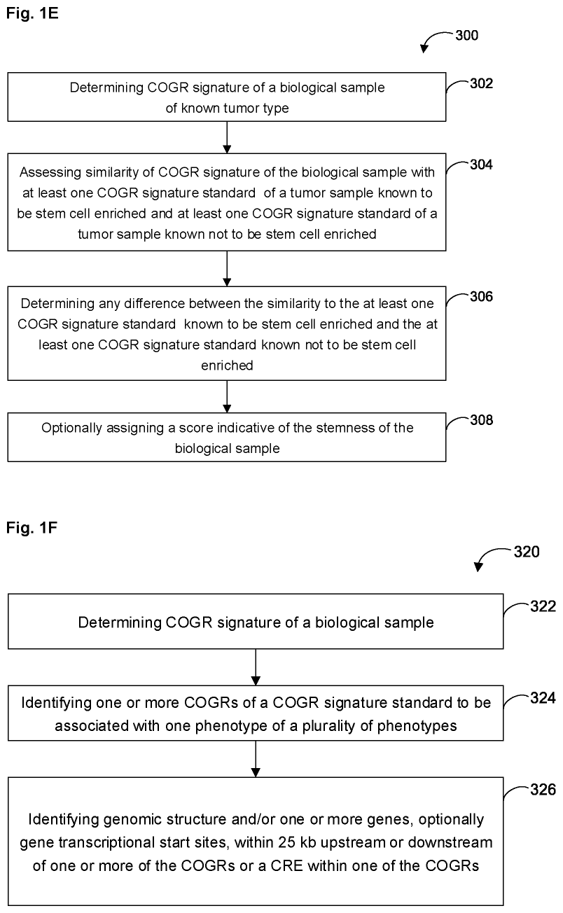

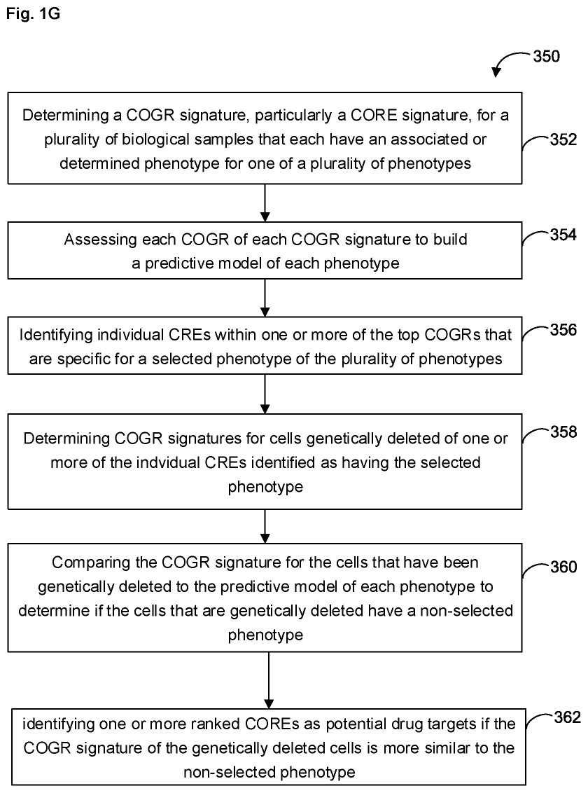

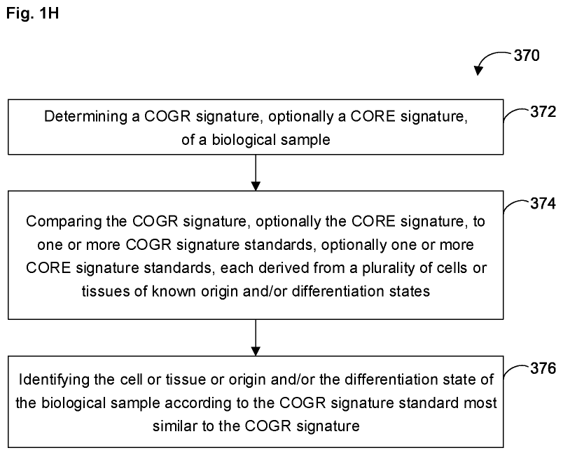

Referring now to FIG. 1A, shown therein is an example embodiment of a genomic regions analysis system 100 for performing one or more analyses based on clusters of genomic regions, with several of the analysis techniques discussed in further detail herein. The system 100 includes a computing device 110 having a processor 112, I/O devices 114, a communication interface component 116 and a storage component 118. The storage component 118 includes various software programs and electronic files including an operating system 120, a cluster of genomic region (COGR) application 122, a CREAM software (s/w) module 124, one or more of a prognostic biomarker discovery software module 126, a prognosis software module 127, a stemness cell identifier software module 128, a genomic or gene analysis software module 129, a drug target identifier software module 130, a tissue of origin identifier software module 131, and an Input/Output (I/O) software module 132 and data files 134. In other embodiments, any of the prognostic biomarker discovery software module 126, prognosis software module 127, the stemness cell identifier software module 128, genomic or gene analysis software module 129, the drug target identifier software module 130, tissue of origin identifier software module 131 may not be included. The software modules 124, 126, 127, 128, 129, 130 and 131 include software instructions for performing certain methods as is discussed in further detail herein. Furthermore, as shown in FIG. 1A, the computing device 110 can be in communication with an external storage component 140 and a user device 150 via a network 160. Alternatively, in other embodiments, the computing device 110 may be a standalone device. In other embodiments, the system 100 and/or the computing device 110 may be implemented differently and may include other components as is known by those skilled in the art.

As shown in the figures and discussed herein, example embodiments of the devices, systems and methods described in accordance with the teachings herein are implemented using a combination of hardware and software. Accordingly, these embodiments may be implemented, at least in part, by using one or more computer programs, executing on one or programmable devices comprising at least one processing element and at least one data storage element (including volatile memory, non-volatile memory, other data storage elements or a combination thereof), and at least one communication interface and/or I/O device. For example, and without limitation, the programmable devices (referred to herein as computing devices or user devices) may be, but is not limited to, a server, a network appliance, an embedded device, a personal computer, a laptop, a personal data assistant, a smart-phone device, a tablet computer or any other computing device capable of being configured to carry out the methods described herein.

The program code may be applied to input data to perform various functions described herein and to generate output data. The output data may be sent to one or more output devices or communicated to another device for display to a user. Each program may be implemented in a high level procedural or object oriented programming and/or scripting language, or both. However, in some cases the programs may be implemented in assembly or machine language, if desired. In any case, the language may be a compiled or interpreted language.

Each computer program may be stored on a storage media or a device (e.g. ROM, magnetic disk, optical disc, a USB key) (i.e. storage element 118) that is readable by the computing device 110, for configuring and operating the computing device 110 to operate as a special purpose programmable computer when the storage media or device is read by the processor 112 of the computing device 110 to perform one or more of the methods described herein. The storage component 118 may also be considered to be a non-transitory computer-readable storage medium that stores various computer programs, that when executed by the computing device 110, causes the computing device 110 to operate in a specific and predefined manner to perform at least one of the methods described in accordance with the teachings herein.

Furthermore, the computer programs implementing the various methods of the embodiments described herein are capable of being distributed in a computer program product comprising a computer readable medium that bears computer usable instructions for one or more processors. The medium may be provided in various forms, including non-transitory forms such as, but not limited to, one or more diskettes, compact disks, tapes, chips, and magnetic and electronic storage media as well as transitory forms such as, but not limited to, wireline transmissions, satellite transmissions, internet transmission or downloads, digital and analog signals, and the like. The computer useable instructions may also be in various forms, including compiled and non-compiled code.

The processor 112 may be any suitable processor, controller or digital signal processor that provides sufficient processing power depending on the configuration, purposes and requirements of the computing device 110. In some embodiments, the processor 112 may be replaced with two or more processors with each processor being configured to perform different dedicated tasks. The processor 112 controls the operation of the computing device 110. For example, the processor 112 can execute the COGR application 122 to allow a user to perform functions and/or analyses provided by one of the software modules 124 to 131.

The I/O devices 114 include at least one input device and one output device for the computing device 110. For example, the I/O devices 114 can include, but is not limited to, a mouse, a keyboard, a touch screen, a thumbwheel, a track-pad, a track-ball, a card-reader, a microphone, a display, a speaker and/or a printer depending on the particular implementation of the computing device 110.

The communication interface component 116 may be any hardware interface that works with corresponding software to enable the computing device 110 to communicate with other devices and systems. For example, the communication interface component 116 may receive input data from or send output data to various devices. In some embodiments in the communication interface may be a software communication interface, such as those for inter-process communication (IPC). In some embodiments, the communication interface component 116 can include, but is not limited to, a serial port, a parallel port and/or a USB port, for example. The communication interface component 116 also generally includes a network communication device such as, but not limited to, a network adapter, such as an Ethernet or 802.11x adapter, a modem or digital subscriber line, a BlueTooth radio or other short range communication device, a Local Area Network (LAN) device, an Ethernet device, a Firewire device, a modem, and/or a digital subscriber line device, for example. In some embodiments, the communication interface component 116 may include a long-range wireless transceiver for wireless communication. For example, the long-range wireless transceiver may be a radio that communicates utilizing CDMA, GSM, or GPRS protocol according to standards such as IEEE 802.11a, 802.11b, 802.11g, 802.11n or some other suitable standard. Various combinations of these elements may be incorporated within the interface component 116.

The storage component 118 can include RAM, ROM, one or more hard drives, one or more flash drives or some other suitable data storage elements such as disk drives, etc. As mentioned the storage component 118 is used to store program files for the operating system 30, the analysis application, and the software modules 124 to 132. The storage component 118 can also store one or more data files some of which might be arranged to provide one or more databases or file system(s). The operating system 120 provides various basic operational processes for the computing device 110. In other embodiments, the software programs may be organized differently but generally provide the same functionality. The modules 126 to 131 may be optional and some of these modules may not be included in certain embodiments.

The COGR analysis application software module 122 allows a user to perform various types of analyses and identification of clusters of genomic regions (COGRs), which includes, but is not limited to COREs and LOCKs, for example. This CRE clustering may result in the generation of a COGR signature of a biological sample (e.g. test sample) or in the generation of a COGR signature standard based on one or more reference biological samples, such as samples obtained from databases 145a to 145m. This generally includes obtaining input chromatin profiles and identifying the genomic regions of interest, applying a clustering method to the genomic regions such as by using the CREAM software module 124 to first apply the CREAM method 200 and then optionally using the output results of the CREAM method for certain purposes such as, but not limited to, similarity analysis, prognostic biomarker discovery, prognosis, stemness cell identification, genomic or gene analysis, drug target identification and/or tissue of origin identification, which may be performed by executing the prognostic biomarker discovery software module 126, the prognosis software module 127, the stemness cell identifier software module 128, the genomic or gene analysis software module 129, the drug target identifier software module 130, and/or the tissue of origin identifier software module 131 respectively. Examples of the methods employed by the CREAM software module 124, the prognostic biomarker discovery software module 126, the prognosis software module 127, the stemness cell identifier software module 128, the genomic or gene analysis software module 129, the drug target identifier software module 130 and the tissue of origin identifier software module 131 are described in more detail with respect to FIGS. 1B, 1C, 1D, 1E, 1F, 1G and 1H, respectively. For example, after obtaining the COGR signature and the COGR standard signature, the stemness cell identifier software module 128 may be used to assess the similarity of the COGR signature of the biological sample with at least one COGR signature standard of known to be stem cell enriched and at least one COGR signature standard known not to be stem cell enriched. The biological sample assessed for its CORE signature can for example be a tumor sample.

The I/O software module 132 receives input data that was obtained by one of the I/O devices 114 or the communication interface 116 and stores the data in the data files 134. The I/O software module 132 also generates output data that may then be sent to the I/O devices 114, such as a display, for output to the user or sent via the communication interface 116 to another device.

The data files 134 may store any temporary data (e.g., data that is not needed after performing any of the methods or steps described herein) or permanent data (e.g., data saved for later use) such as, but not limited to, sample data, and analysis results. The data files 40 may also include parameter values that are used by the various software modules 124 to 131 in performing the methods 200, 250, 270, 300, 320 or 350 or alternative embodiments thereof. At least some data files may also include input data files with chromatin profile data, optionally chromatin accessibility profile data or histone modification data that are used as input to the CREAM module 124. At least some of the data files may also include output data files that are produced by any one of the modules 126 to 131 such as output data files that include COREs, or other COGRs, prognosis or other association data or any combination thereof.

Similar to the storage component 118, the external storage component 140 can include RAM, ROM, one or more hard drives, one or more flash drives or some other suitable data storage elements such as disk drives, etc. The external storage component 140 can include a memory on which one or more databases or file system(s) are stored. Although only one external storage component 140 is shown, there may be multiple external storage components 140 distributed over a wide geographic area and connected via the network 160.

The external storage component 140 can be accessed by the system 100 via the network 160. The external storage component 140 can act as a back-up storage component to the storage component 118 and/or store at least some of the data related to identification of COGRs (e.g. chromosomal position or positions of COREs, LOCKs, CORE signatures, LOCK signatures, etc.) and/or analysis which may include various analysis results and various data catalogues such as genomic region catalogues and associated parameters such as prognostic parameters associated with a genomic region, for example (e.g. COGR signature standards). In some embodiments, the external storage component 140 can store data that is not as frequently used by the system 100, or larger size data. It will be appreciated that the external storage component 140 may additionally store any of the data discussed above with respect to storage component 118. Alternatively, at least some of this data may be stored only on the external storage component 140.

The public databases or repositories 145a to 145m sources such as, but not limited to, the ENCODE database that is publically available from the ENCODE Project (ENCODE Project Consortium 2012), and the ATAC-Seq of the Cancer Genome Atlas (TCGA). The databases 145a to 145m can be accessed by the computing device 110 when certain data is needed for performing any of the methods described herein. For example, this data that is accessed from one of the databases 145a to 145m may include profiles of various samples, such as tumor samples of various types, samples obtained using different assay types, sequencing profiles of various cell lines and the like.

The user device 150 may be any networked device operable to connect to the network 160 in order to communicate with other devices through such as the computing device 110. The user device 150 may include at least a processor and memory, and may be an electronic tablet device, a personal computer, workstation, a server, a portable computer, a personal digital assistant, a laptop, a smart phone, a tablet, and any other mobile electronic device or any combination of these. The user device 150 may couple to the network 160 through a wired or wireless connection.

Although only one user device 150 is shown in FIG. 1A, there may be multiple user devices 150 in communication with the system 100 via the network 160. The user devices 150 can be distributed over a wide geographic area. For example, users in separate cities, provinces, states or countries can use the user device 150 to access the computing device 110. In some embodiments, the computing device 110 may be a server and the user device 150 communicates with the computing device 110 in order to use the COGR application 122, the CREAM software module 124 and optionally one of more of the software modules 126 to 131. For example, the user device 150 may have a chromatin profile as an input file that is transmitted over the network 160 to the computing to the computing device 110 which then performs one or more methods by executing one or more of the software modules 124 to 131 and generates output data that can then be transmitted back to the user device 150 for review by a user of the device. This output data may be displayed on the user device 150 or used in further analysis that is being performed by the user device 150.

The network 160 may be, for example, a wireless local area network such as the IEEE 802.11 family of networks or, in some cases, a wired network or communication link such as a Universal Serial Bus (USB) interface or IEEE 802.3 (Ethernet) network, or others. In some embodiments, the network 160 may be the Internet.

Referring now to FIG. 1B, shown therein is a process flow diagram of an example embodiment of a Clustering of genomic REgions Analysis Method 200 which is referred to as CREAM. The CREAM technique uses genome-wide maps of cis-regulatory elements (CREs) in the tissue or cell type of interest generated from chromatin-based assays such DNase-seq and ATAC-seq and other chromatin based assays such as Ch-IP that are based on histone modifications. CREs can be identified from these data by peak calling tools such as MACS (Feng, Liu, and Zhang 2011). The called individual CREs are then used as input to CREAM. Hence, CREAM does not necessarily need the signal intensity files (bam, fastq) as input from an input data file although various applications will use signal intensity files to create the input data file by performing peak calling on the signal intensity files. CREAM considers proximity of the CREs within each sample to adjust parameters of inclusion of CREs into a CORE or LOCK in the steps described below and herein.

CREAM 200 first obtains a chromatin profile from an input data file at step 210 where the input data file includes the called CREs of a genome as based on the chromatin profile such as the chromatin accessibility profile or the histone modification profile, where the input data file can be obtained from data files 134, storage component 140, from the user device 150, from a portable storage device such as a USB key, from another device that can access the network 160 or from one of the databases 145a to 145m (such as ENCODE for example) for a biological sample that is a test sample for which CREAM 200 determines a COGR signature or for a plurality of known biological samples for which CREAM can determine a COGRs which may later be used to define a COGR signature standard during execution of one of the other software modules. The input data file may be obtained from the storage component 118, the external storage component 140 or provided by the user device 150. Alternatively, the first step 210 of CREAM 200 can be to obtain a signal intensity file, which has signals 212 at different genome regions 214, and then process the signal intensity file by applying a peak calling tool (e.g. MACS) to obtain the called CREs. At this point the input data file is in a Browser Extensible Data (BED) format which is a type of comma separated value (CSV) file format. A BED file is a text file that stores genomic regions using coordinates with associated annotations. For example, the first column in the BED file is used to store a chromosome name for a given chromosome region (e.g. CRE), a second column is used to store the starting position of the given chromosome region and the third column is the ending position of the chromosome region. The number of rows in the BED file is the number of chromosome regions for a biological sample. CREAM 200 then processes the different genome regions 214 to determine COREs 216 that are stored in an output file 218.

The next step 220 in CREAM 200 is to group the individual CREs throughout the genome. At step 220, CREAM 200 groups together different numbers of neighboring individual CREs throughout the genome in the input data file. Therefore, each group of CREs can have a different number of individual CREs. The groups of CREs are categorized based on the number of CREs included in each group. A parameter called Order (O) is defined for each group of CREs. For a given group of CREs the corresponding order O is the number of CREs included in the given group.

One way of generating the groups of CREs is to use a sliding window where the size of the window is the number of CREs to be included in the group. The groups of CREs can therefore be systematically generated for a number of different orders. For example, the order O can be defined to be 2 and the window size is set to 2 so the first and second CREs are placed into group 1, the window is slid over one position and the second and third CREs are placed into group 2, and so on until the last CRE is included in a group. The order O is then increased by one and groups of CREs are formed starting at the first CREs so the first, second, and third CREs are placed into another group, the window is slid over one position and the second, third and fourth CREs are placed into another group and so on. This process is continued for form a plurality of groups of CREs until a maximum order (Omax) is reached, which is determined by performing step 220 along with steps 222 and 224 in a somewhat recursive fashion.

Step 222 of CREAM 200 includes performing maximum window size identification when the groups of CREs have been defined for at least two orders O (e.g. order 2 and order 3). A statistical parameter called the maximum window size (MWS) is defined as the maximum allowed distance between individual CREs included in a given CORE for a given order O. For each order O, CREAM 200 estimates a window size for all groups of CREs within the order O to generate a distribution of window sizes. A window size (WS) is defined as the maximum distance between individual CREs in a group of CREs. Therefore, step 220 will generate a set of window sizes for each order O within the genome. Therefore, for a given order O that has G groups of CREs there will be G window sizes in the set of window sizes for the given order O. The number of sets of window sizes is the same as the largest order O. Afterward, for each order O, the MWS is identified, which may be performed based on determining a low stringent outlier threshold as follows:

MWS=Q1(log(WS))−1.5*IQ(log(WS))

where MWS is the maximum allowed distance between neighboring individual CREs within a CORE (i.e. group of CREs). The parameters Q1(log(WS)) and IQ(log(WS)) are the first quartile and interquartile of distribution of window sizes (see step 2 in FIG. 1B).

The above approach is suggested as the proper value for identification of COREs using chromatin accessibility profiles of cells or tissue. However, in some embodiments, CREAM 200 may be used for identifying clusters of genomic regions for different chromatin profiles (e.g. ATAC-seq and ChIP-seq profiles) of cells or tissues. In order to compare the clusters of CREs (i.e. COREs) across different profiles, the difference between the sizes of individual elements may be considered to improve this comparison. Hence, instead of using the parameter WS for determining MWS, the parameter WS is normalized to the average sizes of the individual elements (e.g. genomic regions) within a particular group of CREs and this normalized parameter WSnormalized for each group of CREs can be determined according to:

$$W {S}_{normalized} = max ( \frac{\begin{matrix}distance between two consecutive \\ elements in the group of CREs\end{matrix}}{\begin{matrix}average size of the two consecutive \\ elements in the group of CREs\end{matrix}} ) ;$$

wherein the distance between two consecutive elements is the distance between an end position of a first CRE of the consecutive elements and a start position of a second CRE of the consecutive elements and where a size of an element (e.g. CRE) is the difference between the ending point and the starting point of the element (e.g. CRE). The MWS can then be determined according to:

MWS=Q1(log(WS_{normalized}))−1.5*IQ(log(WS_{normalized}))

Step 224 of CREAM 200 includes performing Maximum Order identification. After determining the MWS for each Order O of COREs, CREAM 200 identifies the maximum O (Omax) for the given sample (i.e. input data file). Increasing the order O of COREs results in a gain of information within the clusters of CREs, allowing the individual CREs to have further distance from each other within a given CORE. Hence, starting from COREs defined for order O=2, the order O increases up to a plateau at which point an increase of order O does not result in an increase in MWS. This threshold is considered as the maximum order Omax for COREs within the given sample.

It should be noted that steps 220, 222 and 224 are performed iteratively until the maximum order Omax is found. For example, step 220 can be performed to obtain two sets of CORES for order O=2 and order O=3. Step 220 is then performed to determine the MWS for orders O=2 and O=3 which can be represented by MWS2 and MWS3 respectively. If step 224 determines that the MWS3 for Order O=3 is larger than the MWS2 for Order O=2, then CREAM 200 returns to step 210 where the set of CORES for Order O=4 is determined. Step 230 is then performed to determine MWS4 for Order O=4. If MWS3 is less than MWS2 then the maximum order Omax=2. Step 240 is then performed to determine whether MWS4 is larger than MWS3. If this is true CREAM returns to step 210 to determine the set of CORES for Order O=5. If this determination is not true (i.e. MWS4<MWS3), then the maximum order Omax=3. Once the maximum order Omax is found CREAM 200 proceeds to step 250.

Step 226 of CREAM 200 involves performing calling of potential COREs in which CREAM 200 starts to identify COREs from orders Omax down to O=2. For each order O, step 226 involves calling groups of CREs with window size less than MWS as COREs. As a result, many COREs with lower Os are clustered within COREs with higher Os. For example, starting at order=Omax, the maximum window size of each CORE in the set of COREs for order=Omax are compared to the MWSMAX for order=Omax and the COREs with a maximum window size larger than MWSMAX are discarded. Step 226 then considers the set of COREs for the next lower order (i.e. order=Omax−1) and the COREs with a maximum window size larger than MWSMAX-1 are discarded. This continues until the order O=2. After the filtering performed in step 350 of CREAM 200, the remaining lower O COREs, for example O=2 or 3, have individual CREs with distances close to MWS (see step 250—in FIG. 1B). These groups of CREs may have been previously identified as COREs because of the initial distribution of window sizes MWS derived mainly by COREs of the same order O which are clustered in COREs of higher orders Os. CREAM 200 then eliminates these low order O COREs as described in step 228.

It should be noted that the inventors have determined that in a given order O, COREs with a maximum window size (based on physical spacing between CREs within the COREs) that is smaller than the statistical maximum window size MWS for the given order result in more tightly packed COREs that may have more important information in using the COREs for various applications such as prognostic biomarker discovery, cancer stem cell identification, and/or drug target identification.

Step 228 of CREAM 200 involves performing Minimum Order identification. At step 228, COREs that contain individual CREs with distance close to MWS can be identified as COREs due to the high skewness in the initial distribution of MWS. To avoid reporting these COREs, CREAM 200 filters out the clusters with (O<Omin) which does not follow a monotonic increase of maximum distance between individual CREs versus O (see step 360 in FIG. 1B). CREAM 200 starts from the lowest order (O=2) and checks the changes in (MWS-median(WS))/median(WS) where WS is the distribution of maximum distance between individual CREs within COREs of that order. It is possible that other statistical equations can be used to determined MWS. Then CREAM 200 filters out all of the called potential COREs with Order O=2 up to the point where this parameter ((MWS-median(WS))/median(WS) is decreasing when the order O is increased. Step 228 has the effect of filtering out the lower order COREs. This filtering has the effect of removing lower order COREs that are considered to be artifacts from the initial distribution of COREs. This removal of COREs is done for the entire lower order and not just for some COREs of the lower order.

The output of CREAM 200 is an output data file 218 that includes a set of COREs for the biological sample from which the input file was derived. This set of COREs, or set of groups of CREs, is the CORE signature for the biological sample.

Referring now to FIG. 1C, shown therein is a flow chart of an example embodiment of a biomarker discovery method 250 in accordance with the teachings herein. The method 250 may be performed by at least one processor of the computing device 110 when certain program code instructions are being executed. At step 252 of the method 250, CREAM 200 is used to identify of clusters of cis-regulatory elements (COREs) from input data files obtained for a plurality of tumor samples, such as each ATAC-seq profile of the plurality of tumor samples provided in the TCGA dataset, to generate output files containing the CORE signatures for the plurality of tumor types. The plurality of tumor samples have associated data such as prognostic data. The data can be survival.

Then the method 250 proceeds to step 254 where each CORE (chromosomal region) that is located in each CORE signature is assessed and used to generate a univariate predictive model based on the available associated data for a given tumor type. For example, for sets of samples having associated survival data, each CORE that is located for the set of samples, is assessed for its association with the survival (e.g. the probability of a short or long survival) which is determined from the set of samples over time. A given CORE may be used as a binary feature for a each biological sample that is being assessed using in that the CORE exists (i.e. a “1” is used for the feature) or does not exist (i.e. a “0” is used for the feature) and then the existence of the COREs over a plurality of samples for a given tumor type is examined to identify which COREs are predictive of a prognosis such as survival for at least 2 years, at least 3 years etc.

Accordingly, the univariate model determined for a given CORE in step 254 has an associate risk prediction and this can be determined for different tumor types. For example, the D index (which is a robust estimate of the log hazard ratio), and is part of survcomp R package (version 1.38.0), may be used for determining the risk prediction of the univariate model for a given CORE for several tumor types. The hazard ratio is a ratio of the risk outcome in one group to the risk of outcome in another group, for a given time interval. In an alternative embodiment, the risk prediction can be determined using the coph function in R which implements the Cox regression algorithm. In this embodiment, the features and survival time and event may be the same as when using the D index. However, the Cox regression algorithm does not report any p-value. So the COREs (features) can be ranked using the hazard ratio which is one of the outputs of the Cox regression algorithm.

The method 250 then proceeds to step 256 where the COREs are ranked based on their associated risk predictions. For example, the individual COREs can be ranked using the risk prediction based on the significance (p-value) of the risk predictions. The p-value may be determined using the score (logrank) test whether the balanced hazard ratio is different from 1. The logrank test (also known as the log-rank test) can be used to compare the survival distributions of two samples. The logrank test may be used when the data is right skewed and censored (e.g. non-informative). The logrank test may also be called the Mantel—Cox test.

Therefore, in some embodiments, the top COREs can be determined as being the COREs with a minimum FDR (False Discovery Rate) after significance after multiple hypothesis correction with dindex>1 (where dindex is a measure of hazard). The FDR indicates the significance or effect of association between existence (in the case of binary features using COREs) or values of that feature and the output value (like drug sensitivity in case of biomarker discovery). The FDR is a corrected p-value for multiple hypothesis. Then the COREs that exist in less than N samples may be further filtered. The value of N may be 2 or more and depends on the particular COREs and the prognosis being predicted. For some studies described herein N was chosen to be 5. As an optional step, the remaining COREs may be combined to result in the CORE signatures that may be used for a multivariate model. In some embodiments, Fold Change may be used instead of FDR in which case for each CORE, the average drug sensitivity of the samples with that CORE can be divided by the average drug sensitivity of the samples without that CORE. Then the COREs can be sorted based on the obtained fold changes and the COREs with the top fold changes can be selected.

In some embodiments, the method 250 may then proceed to step 258, which is optional, and where the top COREs are combined to generate a multivariate prediction model, examples of which are shown in FIG. 13. For example, for the first graph in FIG. 13, the multivariate model uses 11 COREs and it is seen that the probability of survival over time is higher for people with samples that have two or more of the top identified COREs while the probability of survival over time is lower for people with samples that have one or none of the top identified COREs.

Referring now to FIG. 1D shown therein is a flow chart of an example embodiment of a prognostic method 270 in accordance with the teachings herein. The method 270 may be performed by at least one processor of the computing device 110 when certain program code instructions are being executed. Step 272 of the method 270 includes determining, using CREAM 200, a COGR signature, optionally a CORE signature, of a biological sample previously acquired from a patient. Step 274 of the method 270 includes comparing the COGR signature, optionally the CORE signature, of the biological sample to one or more COGR signature standards, optionally CORE signature standards, associated with a certain outcome. The COGR signature standard may be identified using method 250. Step 274 of the method 270 includes providing the patient with a prognosis according to an associated outcome of the COGR signature standard, optionally the CORE signature standard, with a greatest similarity to that of the biological sample.

Referring now to FIG. 1E shown therein is a flow chart of an example embodiment of a stemness cell identifier method 300 in accordance with the teachings herein. The method 300 may be performed by at least one processor of the computing device 110 when certain program code instructions are being executed. Step 302 of the method 300 includes determining, using CREAM 200, a COGR signature of a biological sample optionally wherein the biological sample is of a known tumor type. Step 304 of the method 300 includes assessing the similarity of the COGR signature of the biological sample with at least one COGR signature standard of known to be or associated with being stem cell enriched and assessing the similarity of the COGR signature of the biological sample with at least one COGR signature standard known not to be or associated with lacking being stem cell enriched. Step 306 of the method 300 includes determining any difference between the similarity of the COGR signature of the biological sample to the at least one COGR signature standard known to be stem cell enriched and the similarity of the COGR signature of the biological sample to the at least one COGR signature standard known not to be stem cell enriched. Step 308 of the method 300 is optional and includes assigning a score indicative of the stemness of the biological sample.

Referring now to FIG. 1F, shown therein is a flow chart of an example embodiment of a genomic or gene analysis method 320 in accordance with the teachings herein. The method 320 may be performed by at least one processor of the computing device 110 when certain program code instructions are being executed. Step 322 of the method 320 includes determining, using CREAM 200, a COGR signature of a biological sample. Optional step 324 of the method 320 includes identifying one or more COGRs to be associated with one phenotype of a plurality of phenotypes. Step 326 of the method 320 includes querying genomic data upstream or downstream of one or more selected COGRs and identifying a genomic structure and/or one or more genes, optionally gene transcriptional start sites, within for example 25 kb upstream or downstream of one or more of the COGRs or a CRE within one of the COGRs. This can provide biological pathway information or other information associated with the COGR, which may be particularly useful for COGRs associated with a phenotype (e.g. a COGR of a COGR signature).

Referring now to FIG. 1G, shown therein is a flow chart of an example embodiment of a drug target identifier method 350 in accordance with the teachings herein. The method 350 may be performed by at least one processor of the computing device 110 when certain program code instructions are being executed. Step 352 of the method 350 includes determining, using CREAM 200, a COGR signature, particularly a CORE signature, for a plurality of biological samples that each have an associated or determined phenotype for one of a plurality of phenotypes. Step 354 of the method 350 includes assessing each COGR of each COGR signature to build a predictive model of each phenotype. Step 356 of the method 350 includes identifying individual CREs within one or more of the top COGRs that are specific for a selected phenotype of the plurality of phenotypes. Step 358 of the method 350 includes determining COGR signatures for cells genetically deleted of one or more of the individual CREs identified as having the selected phenotype. Step 360 of the method 350 includes comparing the COGR signature for the cells that have been genetically deleted to the predictive model of each phenotype to determine if the cells that are genetically deleted have a non-selected phenotype. Step 362 of the method 350 includes identifying one or more ranked COREs as potential drug targets if the COGR signature of the genetically deleted cells is more similar to the non-selected phenotype.

Referring now to FIG. 1H, shown therein is a flow chart of an example embodiment of a tissue of origin identifier method 370 in accordance with the teachings herein. The method 370 may be performed by at least one processor of the computing device 110 when certain program code instructions are being executed. Step 372 of the method 370 includes determining, using CREAM 200, a COGR signature, optionally a CORE signature, of a biological sample. Step 372 of the method 370 includes comparing the COGR signature, optionally the CORE signature, to one or more COGR signature standards, optionally one or more CORE signature standards, each derived from a plurality of cells or tissues of known origin and/or differentiation states. Step 374 of the method 370 includes identifying the cell or tissue or origin and/or the differentiation state of the biological sample according to the COGR signature standard most similar to the COGR signature.

### III. Examples

### Example 1. CREAM Detects COREs from Chromatin Accessibility Profiles

CREAM was developed as a computational approach for the systematic identification of COREs. CREAM is designed to identify COREs from chromatin accessibility profiles through 5 iterative learning steps described in detail in the Examples. Overall, these steps include: 1) Grouping the individual CREs in clusters of varying number of individual CREs (referred to as Order); 2) Identifying threshold for the stitching distance between individual CREs within the clusters of the same Order; 3) Identifying maximum Order of COREs; 4) Clustering individual CREs as COREs starting from the highest Order; and 5) Filtering out low Order COREs with stitching distance close to the corresponding stitching distance threshold of the same Order.

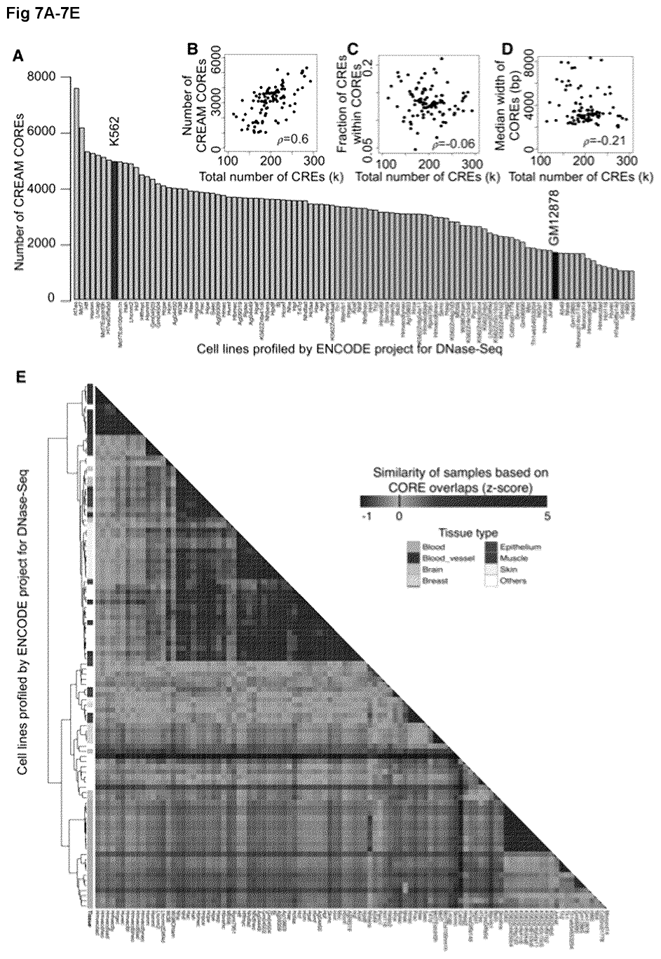

Applying CREAM across the DNase-seq data from 102 cell lines available through the ENCODE project (ENCODE Project Consortium 2012) reveals between 1,022 and 7,597 COREs per cell line (FIG. 7A), correlated with the total number of CREs identified in each cell line (FIG. 7B). However, the fraction of CREs called within COREs is independent of the number of individual CREs (FIG. 7C) and does not impact the median width of COREs across cell lines (Spearman correlation p<0.25; FIG. 7D), supporting the specificity of CORE widths with respect to each biological sample irrespective of the total number of CREs.

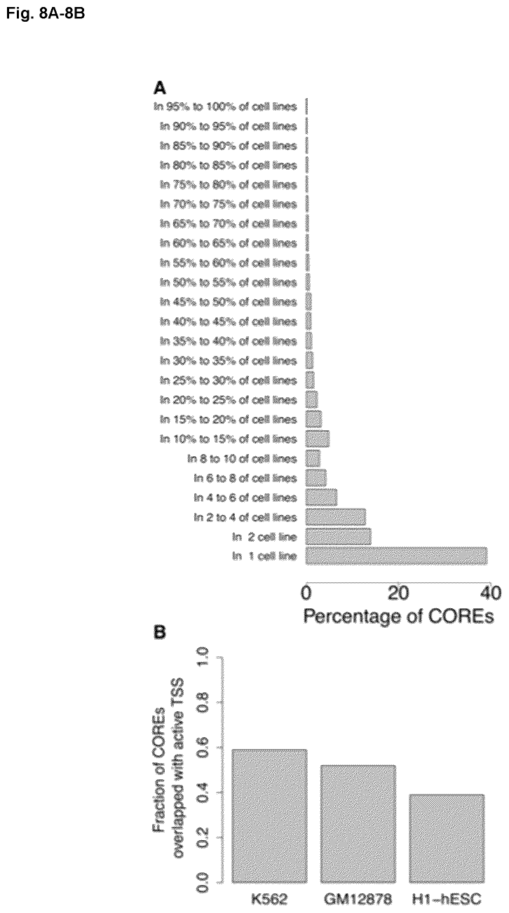

The ability of COREs is shown to classify samples according to their tissue of origin using the ENCODE Project Consortium cell lines. The results described herein specifically show that COREs identify the tissue of origin for the 78 DNase I profiles of the ENCODE Project Consortium cell lines with high accuracy (Matthews correlation coefficient [MCC] of 0.85 for tissues with four or more cell lines) (FIG. 7E). In agreement, close to 40% of the 32,997 COREs found across the ENCODE Project Consortium cell lines are unique to one cell line, and only a very small number are shared across all cell lines (FIG. 8A). Furthermore, even COREs common to >50% of cell lines (12% of all COREs found in the ENCODE Project Consortium cell lines) (FIG. 8) are not enriched at housekeeping genes (P-value>0.05) (Hsiao et al. 2001). Collectively, these results emphasize the cell line specificity of COREs.

The methods used are as described in Example 6.

### Example 2. COREs are Unique Cis-Regulatory Units of Biological Significance

The DNase-seq data from the ENCODE Tier I cell lines (GM12878, K562, and H1-hESC) was used to further characterize the biological underpinning of COREs versus individual CREs. The ENCODE Tier I cell lines were used because of their extensive characterization by the ENCODE project (ENCODE Project Consortium 2012), inclusive of expression profiles and DNA-protein interactions assessed by ChIP-seq assays, allowing for a comprehensive biological assessment of COREs identified across different cell lines.

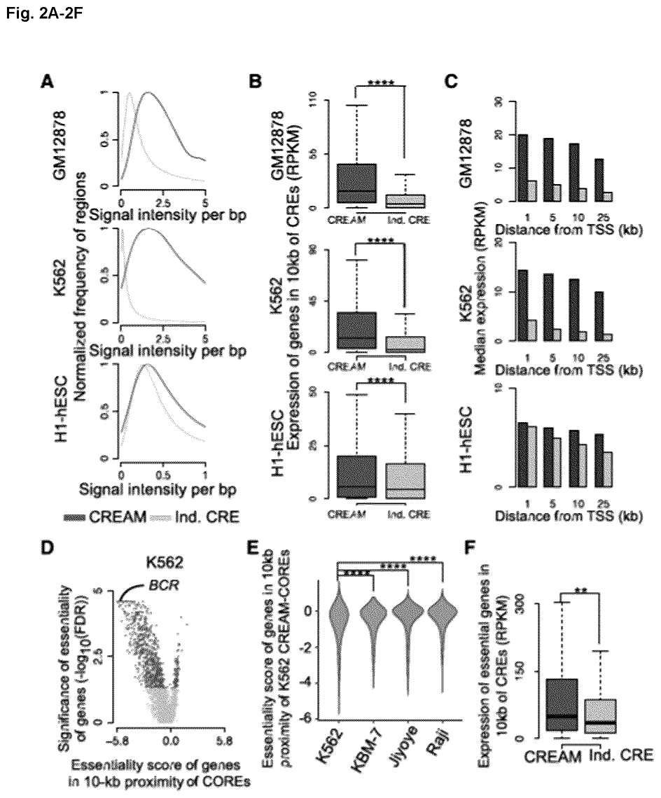

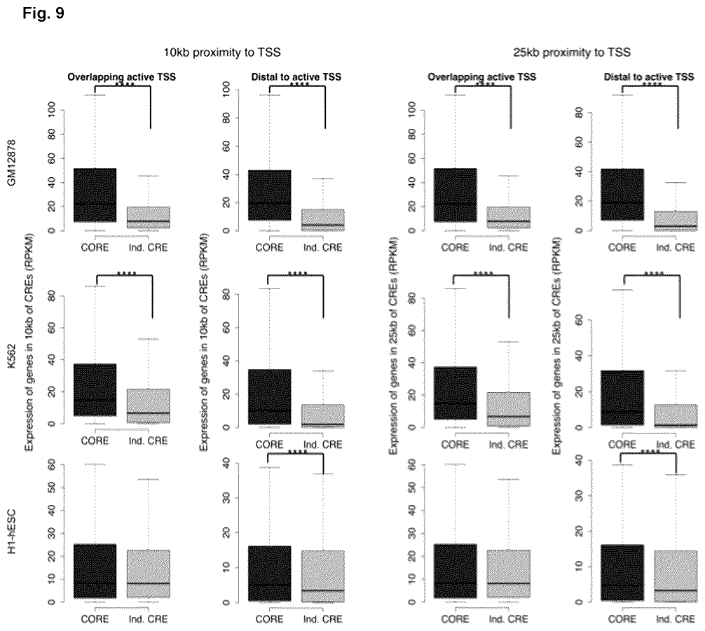

The signal intensity for chromatin accessibility at COREs versus individual CREs was assessed. The results show that COREs have higher average chromatin accessibility signal per base pair compared to individual CREs across the three tested cell lines (GM12878: FC=1.9; K562: FC=8.4; H1-hESC: FC=1.1; FIG. 2A). The difference in the expression level of genes proximal to COREs versus those proximal to individual CREs was examined. COREs are proximal to genes expressed at higher levels than those near individual CREs in the GM12878, K562, and H1-hESC cell lines (Wilcoxon signed-rank test p<0.001; GM12878: FC=4.6; K562: FC=6.8; H1-hESC: FC=1.3; FIG. 2B). Up to 52%, 59%, and 39% of COREs overlap with active transcription start sites (TSSs) (TSSs harboring peaks of chromatin accessibility) in the GM12878, K562, and H1-hESC cell lines, respectively (FIG. 8B). This observation remains significant (p<0.05) even when including genes further away (up to ±25 kb away from transcription start sites (TSS); FIG. 9) from COREs or individual CREs, although differences in the expression of genes proximal to COREs and individual CREs decreases with increasing distance (Spearman correlation p<−0.8; FIG. 2C). Hence, COREs are in proximity of genes with higher expression with respect to genes proximal to individual CREs irrespective of the distance between the CREs and gene TSSs.

The relevance of COREs versus individual CREs in bookmarking genes essential for growth was assessed. For this, the CRISPR/Cas9 gene essentiality screen data reported in the K562 cell line (Wang et al. 2015), was combined with CORE identification from the K562 cell line, revealing the significant enrichment of gene essential for growth proximal to COREs (FDR<0.001 using permutation test; FIG. 2D). This is exemplified at the BCR gene that is the most essential gene and proximal to a CORE in K562 Chronic Myelogenous Leukemia (CML) cell line (FIG. 2D), positive for the BCR-ABL gene fusion reported in CML (Ren 2005). Extending the analysis to essentiality scores from other cell lines tested by Wang et al. (2015) (Wang et al. 2015), shows that the essentiality score of genes proximal to K562 COREs is significantly less in the KBM-7, Jiyoye, and Raja cell lines compared to the K562 cell line (FDR<0.001; FIG. 2E). The expression of genes essential for growth in K562 proximal to COREs is significantly higher than the expression of essential genes associated with individual CREs (FDR<0.001; FIG. 2F). These results support the cell type-specific nature of COREs and their association with essential genes and argue in favor of COREs accounting for a greater regulatory potential relevant to cell type-essentiality than individual CREs.

The methods used are as described in Example 6.

### Example 3: CREAM Identifies COREs Bound by Master Transcription Regulators

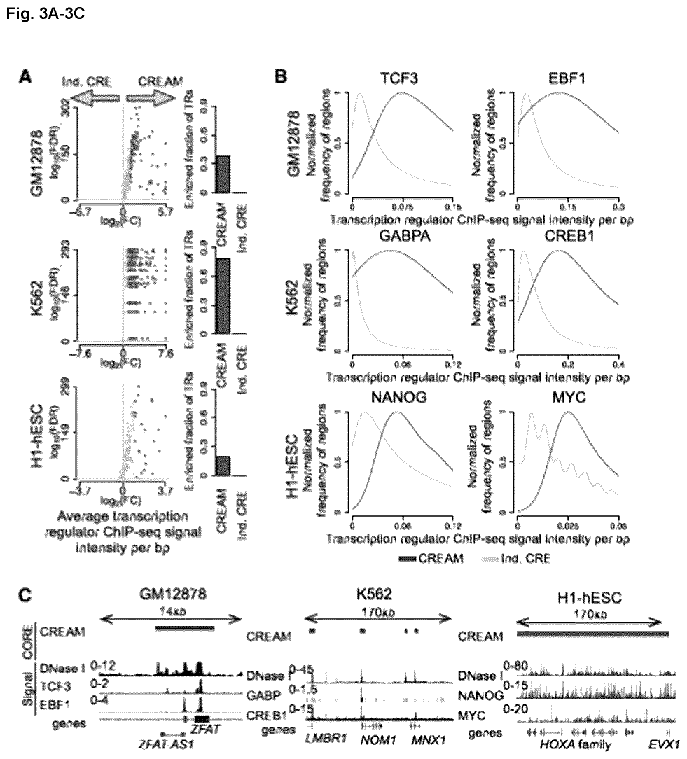

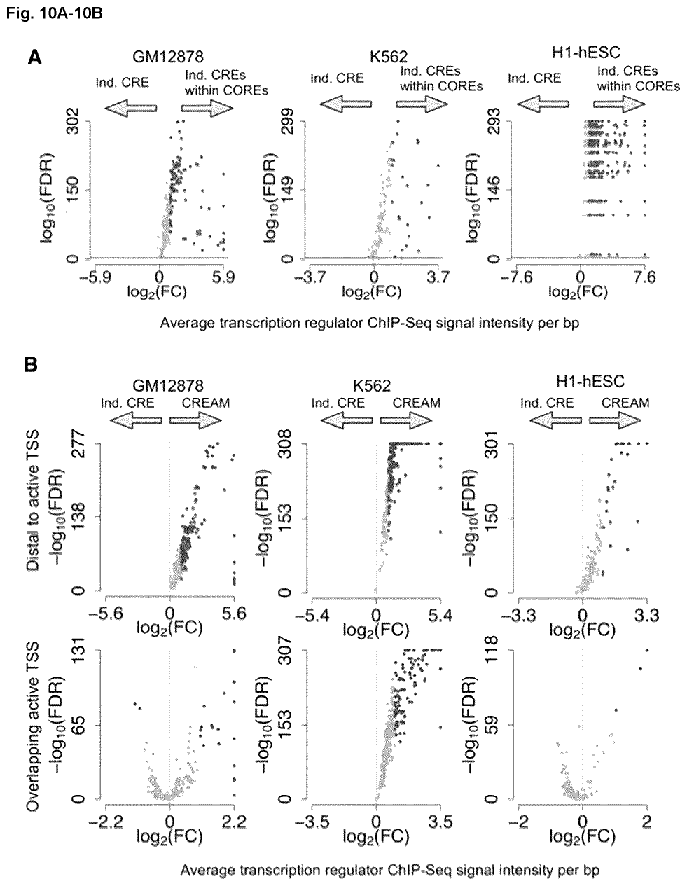

Transcription regulators bind CREs to modulate the expression of cell-type specific gene expression patterns. Quantifying the binding intensity of transcription regulators over COREs in the GM12878, K562 and H1-hESC cell lines reveals that over 20% of ChIP-seq data of transcription regulators (GM12878: 92/237; K562: 256/325; H1-hESC: 24/119) show binding intensity significantly higher over COREs compared to individual CREs, when normalizing the ChIP-seq signal over COREs to the size of each CORE, (FC>2, FDR<0.001; FIG. 3A). The higher enrichment of transcriptional regulators (TR) binding intensity in COREs can be also seen using COREs excluding the CRE-free gaps (FIG. 10A) regardless of whether COREs overlap active TSSs (TSSs harboring peaks of chromatin accessibility) or not (FIG. 10B). This higher transcription regulator binding intensity at COREs is showcased in GM12878 by the master transcription regulators TCF3 and EBF1 (Somasundaram et al. 2015). Specifically, a >3 fold difference in binding intensity for TCF3 and EBF1 in the GM12878 cell line over COREs compared to individual CREs was observed (FIG. 3B), exemplified at the CORE proximal to the ZFAT gene (FIG. 3C). Similarly, the master transcription regulators GABPA and CREB1 (Yang et al. 2013; Shankar et al. 2005) bind with a >3 fold greater intensity over COREs compared to individual CREs in the K562 cell line (FIG. 3B), exemplified at the CORE overlapping the LMBR1, NOMI and MNX1 genes (FIG. 3C). Finally, in the H1-hESC cell line the master transcription regulator NANOG and CMYC (Pan and Thomson 2007) bind with higher intensity at COREs (FC>1.2, FDR<0.001; FIG. 3B) in the H1-hESC cell line, exemplified at the HOXA locus CORE (FIG. 3C).

The methods used are as described in Example 6.

### Example 4: CTCF and Cohesin Enriched COREs Map to Topologically Associated Domain Boundaries

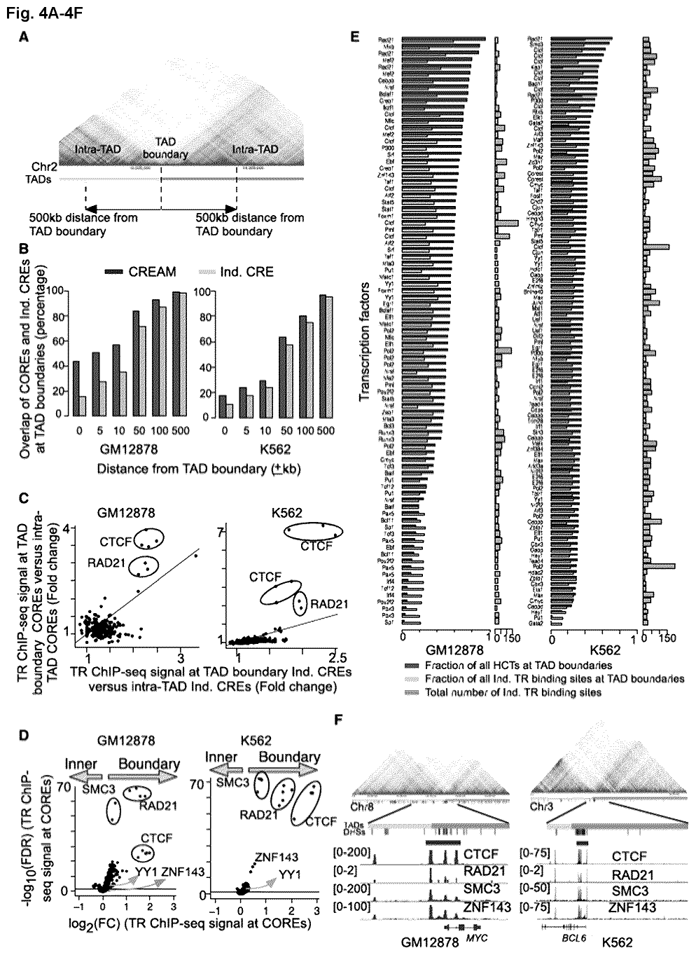

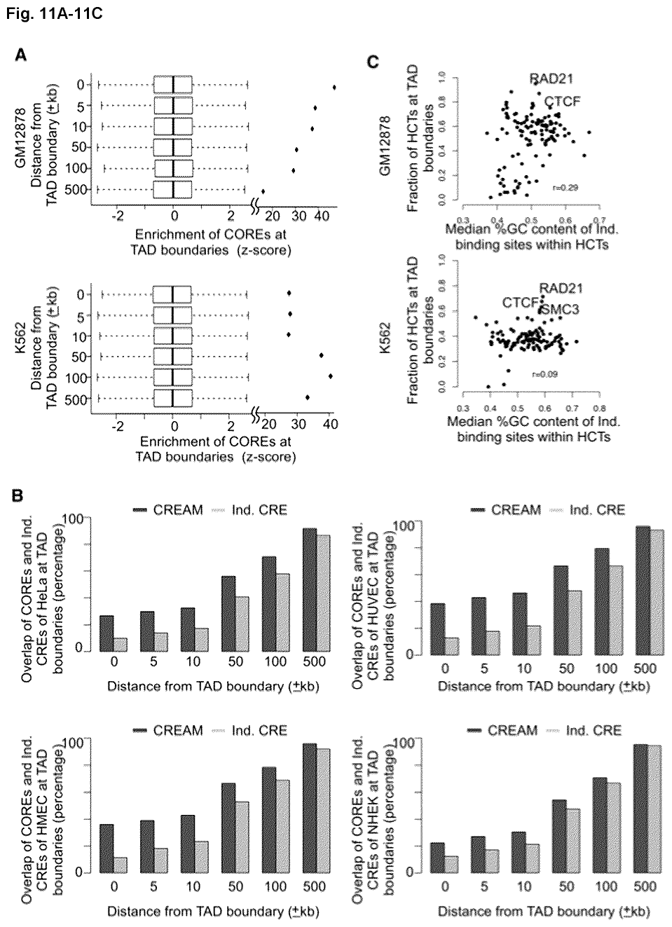

Beyond COREs, the human genome can be partitioned in various clusters including those based on contact frequencies between distal genomic coordinates that define Topologically Associated Domains (TADs) (Ea et al. 2015). To assess the relation between COREs and TADs, the distribution of COREs with TADs reported from HiC data in GM12878 and K562 cell lines was integrated (Rao et al. 2014). The analysis reveals significantly higher fraction of COREs compared to individual CREs at TAD boundaries (permutation test FDR<0.001; FIG. 4A-B, FIG. 11A). Similar results are seen in the HeLa, HMEC, HUVEC, and NHEK cell lines (Ea et al. 2015; Rao et al. 2014) (FIG. 11B). Together, this suggest that COREs are preferentially found at TAD boundaries.

CTCF, cohesin (RAD21, SMC1 and SMC3), YY1 and the ZNF143 transcription regulators preferentially bind chromatin at anchors of chromatin interactions, inclusive of TAD boundaries (Rao et al. 2014; Bailey et al. 2015; Heidari et al. 2014; Weintraub et al. 2017a). The enrichment of these transcription regulators within COREs at TAD boundaries was assessed based on their ChIP-Seq signal intensity. CTCF and RAD21 were preferentially enriched within COREs compared to individual CREs restricted to TAD boundaries in both the GM12878 and K562 cell lines (FC>1.5 for both COREs and individual CREs; FC at COREs more than 1.5 times the FC at individual CREs; FIG. 4C). No enrichment over COREs at TAD-boundaries was seen for ZNF143 and YY1, or any of the 82 and 94 additional transcription regulators with ChIP-seq data in the GM12878 and K562 cell lines, respectively. Together, this argues that CTCF and cohesin behave differently from other transcription regulators at TAD-boundaries, mapping more significantly to COREs as opposed to individual CREs. Furthermore, CTCF and cohesin bind at TAD-boundary COREs with higher intensity than at intra-TAD COREs, defined as COREs within TADs found 10 kb or more away from boundaries, in both the GM12878 and K562 cell lines (FC>2, FDR<0.001 for CTCF and RAD21, FC>1.7, FDR<0.001 for SMC3 in GM12878 and K562 respectively; FIG. 4D). ZNF143 also preferentially occupied TAD-boundary COREs as opposed to intra-TAD COREs but only in the K562 cell line (FC=1.42, FDR<0.001) (FIG. 4D). Lesser differences were observed in the binding intensity of YY1 at TAD-boundary COREs versus intra-TAD COREs in either the GM12878 and K562 cell lines (FC<1.25 in both cell lines; FIG. 4D). Extending this analysis to the remaining ChIP-seq data for transcription regulators in the GM12878 and K562 cell lines (ENCODE Project Consortium 2012), revealed 69% and 35% of transcription regulators with increased binding intensity at TAD-boundary COREs versus intra-TAD COREs but with low effect size in the GM12878 and K562 cell lines, respectively (FC>1, FDR<0.001; FIG. 4D).

The enrichment of CTCF and cohesin within COREs at TAD boundaries suggested that they were themselves forming homotypic clusters of transcription regulator binding regions (HCT) (Gotea et al. 2010) at TAD boundaries. Using CREAM on the 86 and 98 ChIP-seq data from the GM12878 and K562 cell lines, respectively identified 41 and 59 transcription regulators in each cell line forming at least 100 HCTs (Table 1). Comparing the distribution of HCT at TAD boundaries versus intra-TADs revealed that over 50% of CTCF, RAD21, SMC3, and ZNF143 HCT lie at TAD boundaries (FIG. 4E), exemplified at the MYC and BCL6 gene loci (FIG. 4F). This contrasts with other transcription regulators, such as SP1 and GATA2 with fewer than 10% of HCTs mapping to TAD boundaries in the GM12878 and K562 cell lines, respectively (FIG. 4E). The differences in fraction of HCTs at TAD boundaries is not biased to the GC content of the individual binding regions within HCTs (FIG. 11C). Taken together, these results suggest that clusters of CTCF and cohesin binding regions establishing HCTs are preferentially found at TAD boundaries.

The methods used are as described in Example 6.

### Example 5: COREs and Super-Enhancers are Two Distinct Biologically Significant Features of Cells

Similar to COREs, super-enhancers were introduced as high-signal intensity regions identified from ChIP-seq data from features, such as H3K27ac or MED1, typical of a subset of CREs including promoters and enhancers (Hnisz et al. 2013; Vahedi et al. 2015; Lovén et al. 2013). Although the concept of clusters of cis-regulatory elements was introduced before super-enhancers (de Laat and Grosveld 2003; Gaulton et al. 2010; Song et al. 2011), the computational method developed for super-enhancer calling, known as ROSE (Hnisz et al. 2013), accelerated the inclusion of super-enhancer identification across numerous studies. CORE identification by CREAM was therefore compared with super-enhancer mapping from ROSE using the data from the GM12878, K562 and H1-hESC cell lines.

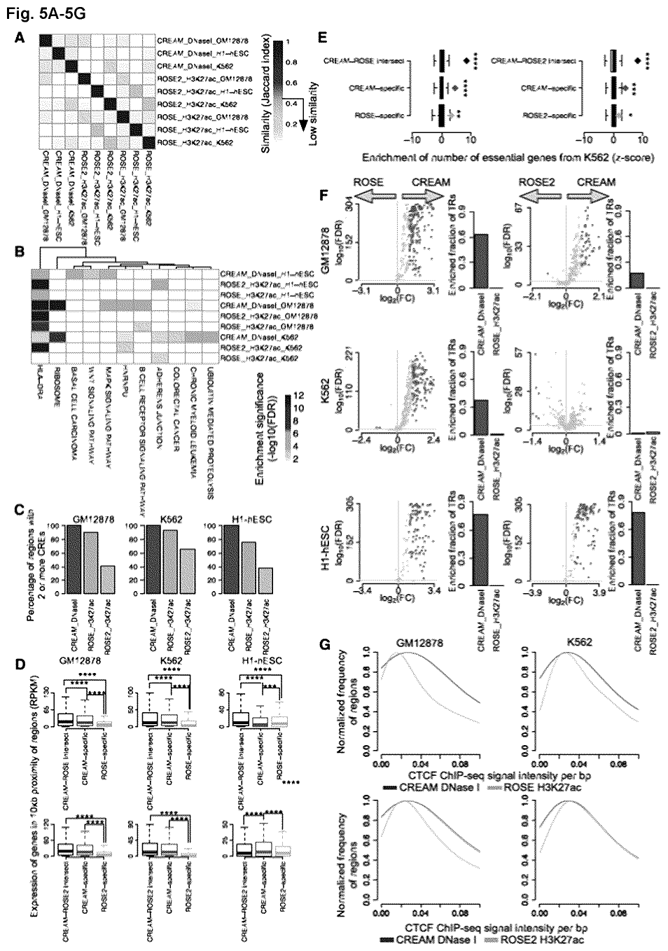

The comparison of super-enhancers, identified either by ROSE or its latest version (ROSE2), with COREs revealed limited overlap in all the three cell lines (Jaccard index<0.5; FIG. 5A). Moreover, the pathway enrichment analysis based on genes within 10 kb of COREs or super-enhancers, shows higher enrichment of phenotypic specific pathways for COREs. For instance, enrichment for the B CELL RECEPTOR SIGNALING PATHWAY term is 2.6 fold more significant based on COREs as opposed to super-enhancers found in the lymphoblastoid GM12878 cell line (FIG. 5B). Similarly, the CHRONIC MYELOID LEUKEMIA term is 3.07 fold more significantly enriched in genes proximal to COREs compared to super-enhancer in the chronic myeloid leukemia K562 cell line (FIG. 5B). Finally, the WNT SIGNALING PATHWAY term is 2.36 fold more significantly enriched in genes proximal to COREs compared to super-enhancers in the H1 human embryonic stem cell line (FIG. 5B).

The structure of COREs and super-enhancer according to their proportion reported to harbor 2 or more CREs was also compared. While all COREs consisted of at least 2 CREs, between 75% and 90% of super-enhancers identified by ROSE were composed of at least two CREs in the GM12878, K562 and H1-hESC cell lines (FIG. 5C). This number plummets to less than 65% for super-enhancers called by ROSE2 in these same cell lines (FIG. 5C). The relationship between gene expression versus COREs and super-enhancers identified by ROSE was compared. The expression of CORE-specific genes were significantly higher in the GM12878 and K562 cell lines compared to genes exclusively in proximity of super-enhancers (FDR<0.001; FIG. 5D). The opposite was seen in the H1-hESC cell line (FDR<0.001; FIG. 5D). Expanding this analysis to genes essential for growth in the K562 cell lines revealed that genes located in proximity of both COREs and super-enhancers have the highest enrichment for essential genes, followed by genes only proximal to COREs and finally genes only proximal to super-enhancers (FIG. 5E). These results highlights differences where COREs are more significantly associated with biological functions than super-enhancers. COREs identified using CREAM are a more precise reflection of cellular identity and function.

As a final comparison, the enrichment of transcription regulators according to their ChIP-seq profiles within COREs versus super-enhancers was assessed. The analysis reveals that over 60% of transcription regulators are significantly enriched in COREs compared to super-enhancers in the GM12878 and H1-hESC cell lines (FC>2 and FDR<0.001; FIG. 5F). In the K562 cell line, over 30% of transcription regulators are more significantly enriched in COREs compared to ROSE-super-enhancers (FC>2 and FDR<0.001; FIG. 5F). In contrast, less than 2% of transcription regulators are more significantly enriched in ROSE-super-enhancers compared to COREs in any of the three cell lines (FIG. 5F). Similar results are obtained with comparing COREs to ROSE2-super-enhancers in the H1-hESC cell line, with lower enrichment reported in GM12878 and K562 cell lines (FC>2 and FDR<0.001) (FIG. 5F). CTCF and the cohesin complex are amongst the transcription regulators preferentially enriched in COREs as opposed to super-enhancers in each cell line tested. The enrichment of CTCF at COREs versus super-enhancers located at TAD boundaries, inclusive of a 10 kb window around these boundaries, was therefore assessed. The analysis revealed the strong binding intensity of CTCF within COREs at TAD boundaries, and weaker binding intensity within super-enhancers at TAD boundaries, in the GM12878 and K562 cell lines (FDR<0.001; FIG. 5G). Collectively, these results support the unique biological nature of COREs compared to super-enhancers towards chromatin looping factors and TAD boundaries, of relevance to the three-dimensional organization of the genome.

### Example 6: Clinical Utility of CREAM to Identify COREs Discriminating Tumor Type and Underpinning Biological Pathway

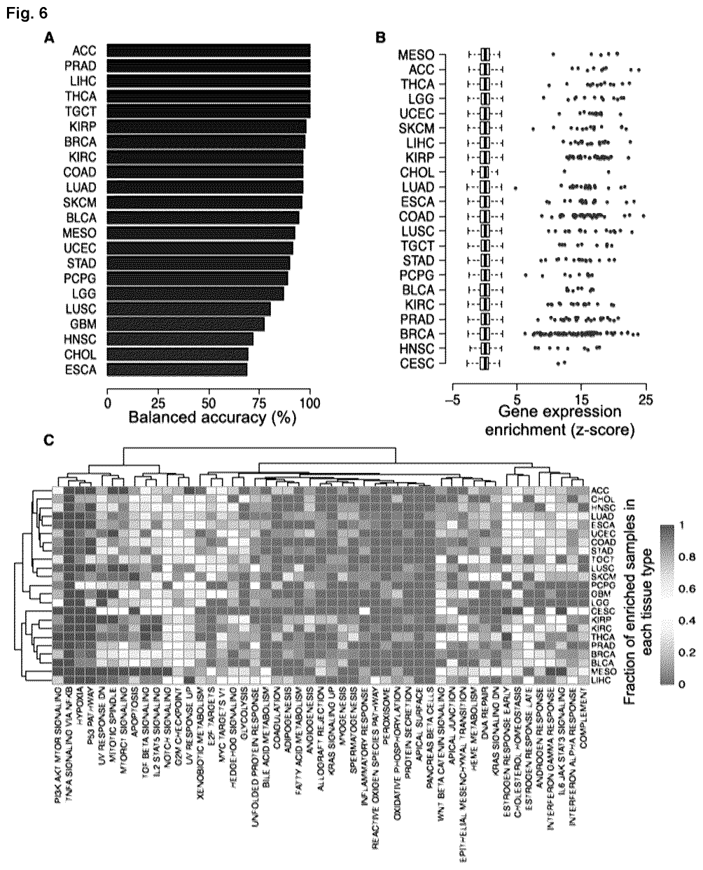

CRE identification in 400 human tumour samples from 23 different cancer types part of The Cancer Genome Atlas (TCGA) was recently completed through ATAC-seq assays (Corces et al. 2018). Using the k-nearest neighbor method (k=3) on the COREs identified by CREAM classified these TCGA ATAC-seq profiles according to their tumor type (MCC=0.86; FIG. 6A). Out of 22 cancer types with more than 4 patient samples with available ATAC-seq profiles, 17 had balanced accuracy of more than 85% (FIG. 6A). In patient tumor ATAC-seq profiles, COREs were located in proximity of genes with higher expression than individual CREs (FIG. 6B) and were significantly over-represented in 49 out of 50 hallmarks of cancer gene sets (FDR<0.05; FIG. 6C) (Liberzon et al., 2015). The TNF-α SIGNALING VIA NF-KB hallmark gene set was enriched for almost all of the TCGA samples, while other hallmark gene sets were tissue-specifically enriched, such as the ANDROGEN RESPONSE hallmark gene set, enriched in prostate adenocarcinoma (PRAD) tumor samples (FIG. 6C). Altogether, these results suggest the utility of COREs in clinical setting to discriminate cancer types and identify hallmark gene sets within each tumor samples of biological relevance.

We developed CREAM as the first computational approach for CORE identification using ATAC-seq profiles of the cells. Applying CREAM on TCGA ATAC-seq profiles, we identified combinations of COREs as prognostic signatures of lung and colon adenocarcinoma.

The identified signature is a combination of COREs each containing a set of individual CREs, open chromatin detected by ATAC-seq profiling of cells. CREAM is a complex multi-step statistical approach for CORE identification. Repurposing of widely used machine learning methods for clustering of genomic regions will not be as effective as CREAM as they have been designed for clustering of samples in distance space not genomic regions in physical space.

Details of the locations/signatures identified in lung and colon adenocarcinomas are provided in FIG. 12. The methods used are described in Example 7.

Although the concept that CREs are not all equal is well established, their classification into clusters is recent and warrants the development of strategies for their classification according to the various approaches developed to map CREs. As described herein, CREAM was developed as the first unsupervised machine learning method providing a systematic approach to set the filters through an iterative learning process to identify COREs from chromatin accessibility profiles generated in any cell type. As shown herein, CREAM identifies COREs that have higher transcription regulator binding intensity and that are enriched proximal to genes essential for growth compared with individual CREs. CREAM-identified COREs also classify cell types according to their tissue of origin, discriminating normal from cancer cells. These results support the utility of CREAM for reporting COREs from chromatin accessibility data of biological significance.

The biological relevance of COREs was assessed with regards to the three-dimensional organization of the genome by comparing their distribution with regard to TADs. The results show that COREs are enriched compared with individual CREs at TAD boundaries. These COREs are preferentially bound by a limited number of transcription regulators, namely, CTCF, the cohesin complex (RAD21, SMC3), and, to a lesser extent, ZNF143. These are transcription regulators previously shown to regulate contact frequencies between distal genomic coordinates defining the three-dimensional organization of the genome (Heidari et al. 2014; Rao et al. 2014; Bailey et al. 2015; Weintraub et al. 2017).

Also shown here, COREs are distinct from super-enhancers defined by ROSE and ROSE2 when relying on DNase-seq data (of note, ROSE and ROSE2 were designed for identifying super-enhancers based on H3K27ac ChIP-seq data). Specifically, COREs show higher enrichment of biological pathways associated with phenotype of each cell type. Moreover, COREs compared with super-enhancers show higher enrichment in proximity of highly expressed and essential genes in binding of transcription regulators and association to CTCF-enriched regions at TAD boundaries.

Finally, the clinical value of CORE identification in 400 tumor samples was shown to delineate their cancer type and enriched biological pathways based on genes proximal to COREs in each sample. In the process, the first pan-cancer CORE data set was derived from 400 publicly released chromatin accessibility profiles (Corces et al. 2018) covering 23 distinct human cancer types. Overall, the results support the relevance of CREAM to classify CREs into COREs, and show the value of COREs, independently on genome assembly version, to delineate the biology unique to any sample profiled for its chromatin accessibility.

### Methods

CREAM: CREAM uses genome-wide maps of cis-regulatory elements (CREs) in the tissue or cell type of interest generated from chromatin-based assays such DNase-seq and ATAC-seq. CREs can be identified from these data by peak calling tools such as MACS (Feng, Liu, and Zhang 2011). The called individual CREs are then used as input of CREAM. Hence, CREAM does not need the signal intensity files (bam, fastq) as input. CREAM considers proximity of the CREs within each sample to adjust parameters of inclusion of CREs into a CORE in the following steps (FIG. 1):

Step 1: Grouping of individual CREs throughout genome. CREAM initially groups neighboring individual CREs throughout the genome. Each group can have different number of individual CREs. Then it categorizes the groups based on their included CRE numbers. We defined Order (O) for each group as its included CRE number. In the next steps, CREAM identifies maximum allowed distance between individual CREs for calling a group as CORE of a given O.

Step 2: Maximum window size identification. The maximum window size (MWS) was defined as the maximum allowed distance between individual CREs included in a CORE. For each O, CREAM estimates a distribution of window sizes, as the maximum distance between individual CREs in all groups of that O within the genome. Afterward, MWS is identified based on the low stringent outlier threshold as follows:

MWS=Q1(log(WS))−1.5*IQ(log(WS))

where MWS is the maximum allowed distance between neighboring individual CREs within a CORE. Q1(log(WS)) and IQ(log(WS)) are the first quartile and interquartile of distribution of window sizes (FIG. 1).

Step 3: Maximum Order identification. After determining MWS for each Order of COREs, CREAM identifies maximum O (Omax) for the given sample. Increasing O of COREs results in gain of information within the clusters, allowing the individual CREs to have further distance from each other. Hence, starting from COREs of O=2, the O increases up to a plateau at which an increase of O does not result an increase in MWS. This threshold is considered as maximum O (Omax) for COREs within the given sample.

Step 4: CORE calling. CREAM starts to identify COREs from Omax down to O=2. For each O, it calls groups with window size less than MWS as COREs. As a result, many COREs with lower Os are clustered within COREs with higher Os. Therefore, remaining lower O COREs, for example O=2 or 3, have individual CREs with distance close to MWS (FIG. 1). These clusters could have been identified as COREs because of the initial distribution of MWS derived mainly by COREs of the same O which are clustered in COREs of higher Os. Hence, CREAM eliminate these low O COREs as follows.

Step 5: Minimum Order identification. COREs that contain individual CREs with distance close to MWS can be identified as COREs due to the high skewness in the initial distribution of MWS. To avoid reporting these COREs, CREAM filters out the clusters with (O<Omin) which does not follow monotonic increase of maximum distance between individual CREs versus O (FIG. 1). CREAM starts from the lowest order (O=2) and checks changes of (MWS-median(WS))/median(WS) where WS is the distribution of maximum distance between individual CREs within COREs of that order. Then CREAM filters out called COREs with Order=2 up to the point where this parameter ((MWS-median(WS))/median(WS) is decreasing by increasing order.

Statistical analysis was conducted in R version 3.5.1 (R Core Team 2018).

Association with genes: A gene is considered associated with a CRE or a CORE if the CRE or CORE is found within a ∓10 kb window from the TSS of the gene. This distance was chosen to avoid false-positive association of elements with gene TSSs based on previous reports (Sanyal et al. 2012), however other distances could be used. Expression of genes with respect to distance of COREs and individual CREs with gene TSSs were conducted for different distances from ∓1 kb up to ∓25 kb window as suggested in Sanyal et al. (2012).

Association with essential genes: Number of genes which are in ∓10 kb proximity of COREs and are essential in the K562 cell line are identified (Wang et al. 2015). This number is then compared with number of essential genes in 10,000 randomly selected (permuted) genes, among the genes included in the essentiality screen. This comparison is used to compute FDR, as number of false discoveries in permutation test, and z-score regarding the significance of enrichment of essential genes among genes in ∓10 kb proximity of COREs identified for the K562 cell line.

Gene expression comparison: RNA sequencing profiles of the GM12878, K562, and H1-hESC cell lines, available in ENCODE database (ENCODE Project Consortium 2012), are used to identify expression of genes in proximity of individual CREs and COREs. Expression of genes are compared using Wilcoxon signed-rank test.

Gene expression enrichment in TCGA: Expression of genes associated with COREs of each tumor sample in TCGA were compared with expression of 100 randomly selected gene sets, with the same number of genes. Z-score is calculated considering the null distribution generated relying on the average gene expression in the random gene sets. The p-values were also calculated comparing the expression of genes associated with COREs with genes associated with individual CREs using Wilcoxon signed-rank test.

Pathway enrichment analysis: Hypergeometric test was used to identify p-values for enrichment of hallmark gene sets using the dhyper function in stats R package. CORE-associated genes for each sample and catalogue of genes associated with peaks were used as query and background gene lists, respectively.

Transcription regulator and input signal binding enrichment: Bedgraph files of ChIP-Seq data of transcription regulators are overlapped with the identified COREs and individual CREs in the GM12878, K562, and H1-hESC cell line using bedtools (version 2.23.0). The resulting signals were summed over all the individual CREs or COREs and then normalized to the total genomic coverage of individual CREs or COREs, respectively. These normalized transcription regulator binding intensities are used for comparing TR binding intensity in individual CREs and COREs (FIG. 4). Wilcoxon signed-rank test is used for this comparison.

Similar analysis is used, for enrichment of transcriptional regulators, to get overlap of DNase I signal data of the cell lines within individual CREs and COREs. The overlapped signal then normalized to the size of COREs and individual CREs. Distribution of these normalized signal per base within COREs and individual CREs were then compared for a given cell line.

The DNase I, ChIP-seq and gene expression profiles available from all three Tier I cell lines from the ENCODE project (GM12878, K562 and H1-hESC) were included to provide a comprehensive analysis of COREs versus biochemical measurements across a diverse collection of cell types, acknowledging that differences in the significance in trends across cell lines could arise from cell type-specific biology or variability in the quality of data between cell types.

Sample similarity: Similarity between two samples in ENCODE or TCGA datasets were identified relying on Jaccard index for the commonality of their identified COREs throughout the genome. Then this Jaccard index was used as the similarity statistics in a 3-nearest-neighbor classification approach. Performance of the classification was assessed using leave-one-out cross validation. Matthews correlation coefficient was used for performance of the classification model (Smirnov et al. 2016). Phenotype of each tissue was considered as a class and the obtained vectors was used to calculate MCC using the implemented MCC function in PharmacoGx package in R (Smirnov et al. 2016). In this classification scheme, phenotype of the closest sample to an out of pool sample as its phenotype was considered.

Multiple hypothesis correction: P-values were corrected for multiple hypothesis testing using the Benjamini-Hochberg procedure (McDonald 2009).

Research reproducibility: CREAM is publicly available as an open source R package on the Comprehensive R Archive Network (https://CRAN.R-project.orq/package=CREAM).

TABLE 1
Number of TR-COREs identified by CREAM for transcription
regulators with ChIP-Seq data in ENCODE project for
GM12878 and K562 and more than 100 TR-COREs.

| GM12878 | | K562 | |
|---|---|---|---|
| | Average number | | Average number |
| transcription | of TR-COREs | transcription | of TR-COREs |
| regulator | among replicates | regulator | among replicates |
| RUNX3 | 1334 | MAFK | 904 |
| CTCF | 1284 | MAFF | 732 |
| ZNF143 | 843 | JUND | 685 |
| EBF1 | 793 | ZNF143 | 673 |
| CMYC | 767 | TEAD4 | 657 |
| RAD21 | 693 | RAD21 | 646 |
| EBF | 677 | CTCF | 568 |
| STAT1 | 593 | PU1 | 544 |
| YY1 | 564 | ELF1 | 514 |
| MTA3 | 531 | MAX | 498 |
| BATF | 428 | HDAC2 | 494 |
| TCF12 | 397 | CBX3 | 481 |
| STAT5 | 388 | CCNT2 | 477 |
| P300 | 385 | BHLHE40 | 470 |
| BCL3 | 384 | HCFC1 | 463 |
| ATF2 | 382 | ZNF384 | 463 |
| PAX5 | 376 | IRF1 | 456 |
| FOXM1 | 375 | SMC3 | 450 |
| PU1 | 371 | TBLR1 | 446 |
| NFIC | 368 | CEBPB | 442 |
| BCLAF1 | 340 | CMYC | 429 |
| NFATC1 | 339 | NRSF | 423 |
| PML | 321 | CDPS | 403 |
| TCF3 | 295 | P300 | 378 |
| SP1 | 272 | ZNFMIZ | 376 |
| ELF1 | 236 | NR2F2 | 364 |
| IRF4 | 232 | ARID3A | 362 |
| IKZF1 | 189 | EGR1 | 352 |
| POU2F2 | 178 | RFX5 | 347 |
| BCL11 | 177 | ZBTB7 | 338 |
| TAF1 | 168 | HEY1 | 321 |
| POL2 | 165 | COREST | 313 |
| MEF2 | 162 | PML | 308 |
| ZEB1 | 156 | ATF1 | 305 |
| PBX3 | 150 | MAZ | 276 |
| NFE2 | 138 | CJUN | 244 |
| CREB1 | 134 | CHD2 | 243 |
| SRF | 132 | CEBPD | 236 |
| CEBPB | 129 | USF1 | 226 |
| EGR1 | 109 | MXI1 | 219 |
| NRSF | 106 | ZC3H1 | 218 |
| NFKB | 103 | TRIM28 | 209 |
| | | E2F6 | 208 |
| | | NFYB | 205 |
| | | STAT5 | 203 |
| | | YY1 | 198 |
| | | KAP1 | 189 |
| | | ELK1 | 187 |
| | | POL2 | 180 |
| | | GABP | 156 |
| | | ATF3 | 156 |
| | | HMGN3 | 154 |
| | | GATA2 | 153 |
| | | BACH1 | 151 |
| | | TAF1 | 148 |
| | | SIN3 | 145 |
| | | ETS1 | 139 |
| | | GTF2 | 129 |
| | | FOSL1 | 110 |

### Example 7: CREAM for Prognostic Biomarker Discovery

Cis-regulatory elements (CREs) control of gene expression provides for cellular identity in normal tissues and can be used to gain insights on disease susceptibility. The uneven distribution of CREs across the genome has made their identification and characterization technically and biologically challenging. CREAM automates the detection of clusters of CREs, and is useful in predicting the severity of certain cancer types. In particular, signatures derived from ATAC-seq data were able to correctly stratify 400 tumor samples according to their cancer type and to delineate cancer type-specific active biological pathways. COREs signatures in Lung and Colon Adenocarcinoma were further characterized and these novel gene signatures have shown potential in predicting disease progression. As shown in Example 6, the Lung Adenocarcinoma signature comprises 2 chromosomal locations, and the Colon Adenocarcinoma signature comprises 7 chromosomal locations. These signatures were derived from ATAC-seq data, and the rapid and ready use of similarly derived profiles represents a key feature for this technology, which will have immediate application in research discovery research, and longer-term applicability to clinical decision making and treatment.

The utility of CREAM to identify CORE signatures for four other tumor types including kidney renal papillary cell carcinoma, stomach adenocarcinoma, liver hepatocellular carcinoma and lung squamous cell carcinoma is shown in FIG. 13. This may be determined by using method 250 in FIG. 1C. Survival rate and ATAC-seq profiles of tumor samples provided in TCGA dataset were used to identify these new signatures.

CREAM was used to identify clusters of cis-regulatory elements (COREs) for each tumor sample using ATAC-seq profile of the sample provided in TCGA dataset. Then each CORE (chromosomal region) was used as a feature to build a univariate predictive model of survival in each tumor type in TCGA. D index (a robust estimate of the log hazard ratio), as part of survcomp R package (version 1.38.0), was used for risk prediction in each tumor type. After ranking individual COREs for risk prediction based on the significance (p-value) of the predictions, we combined the top ones to come up with final multivariate signatures provided in the figures.

Details of the locations/signatures identified in lung and colon adenocarcinomas, kidney renal papillary cell carcinoma, stomach adenocarcinoma, liver hepatocellular carcinoma and lung squamous cell carcinoma are provided in FIG. 13.

### Example 8: Genomic Coverage of Active LOCKs Discriminate ESCs from Mature Phenotypes

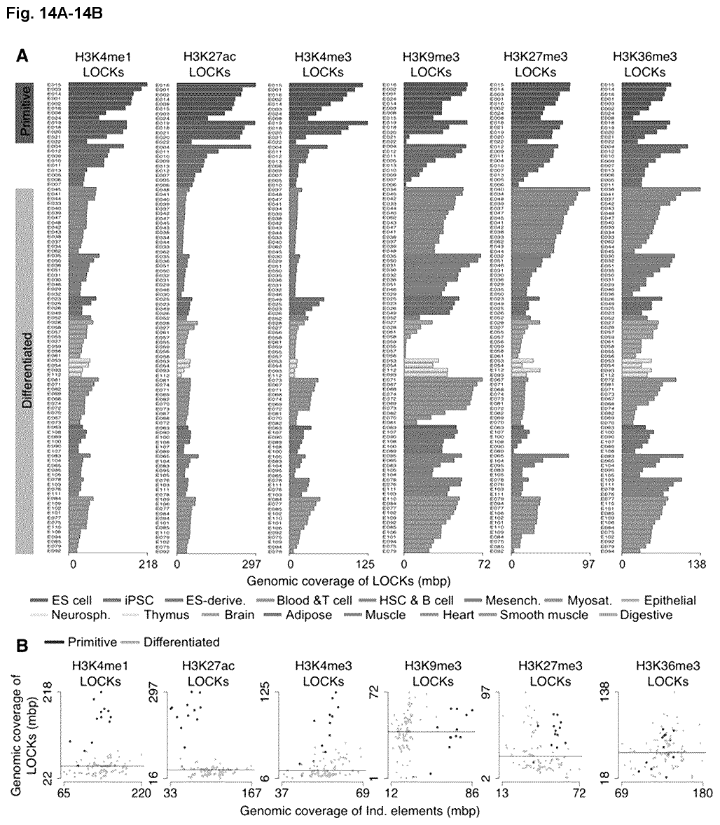

The Roadmap Epigenomics Project released the complete epigenomes (H3K4me1, H3K4me3, H3K27ac, H3K9me3, H3K27me3 and H3K36me3 from ChIP-seq) across 13 primitive cell types, including Embryonic Stem Cells (ESCs) and induced Pluripotent Stem Cells (iPSCs) as well as 9 ES-derived and 77 differentiated cell types from diverse tissue or origin⁶. Expanding previous work comparing ChIP-seq profiles of histone modifications across stem and differentiated cells conducted on individual elements¹⁷, the CREAM tool as described herein for example in Examples 1-6 was used to identify Large Organized Chromatin Lysine (K) domains (LOCKs) across all 99 aforementioned cell types. Overall, LOCKs of active marks including H3K4me1, H3K4me3 and H3K27ac cover a maximum of 297 mbp of the human genome within one cell type, while LOCKs of H3K9me3, H3K27me3 and H3K36me3 repressive marks cover at most 138 mbp of the human genome within one cell type (FIG. 14A). Comparing between cell types, LOCKs of the H3K4me1, H3K4me3 and H3K27ac active marks cover a larger proportion of the genome in primitive cells, including ESCs and iPSCs, compared to differentiated cells (non-ESCs and -iPSCs and -ES-derived; FDR<0.05; Wilcoxon signed-rank test; Fold change>3.1) (FIG. 14A-B). In comparison, the genomic coverage of individual elements for these active histone modifications does not discriminate primitive from differentiated cells (FIG. 14B). In contrast, H3K36me3, H3K27me3 and H3K9me3 derived LOCKs do not show any significant differences in the proportion of the genome covered between primitive and differentiated cells (FDR>0.05; Wilcoxon signed-rank test; Fold change<1.1) (FIG. 14A-B).

The methods used are as described in Example 12.

### Example 9: LOCKs of Active Histone Marks are Predictive of Primitive Cell Identity

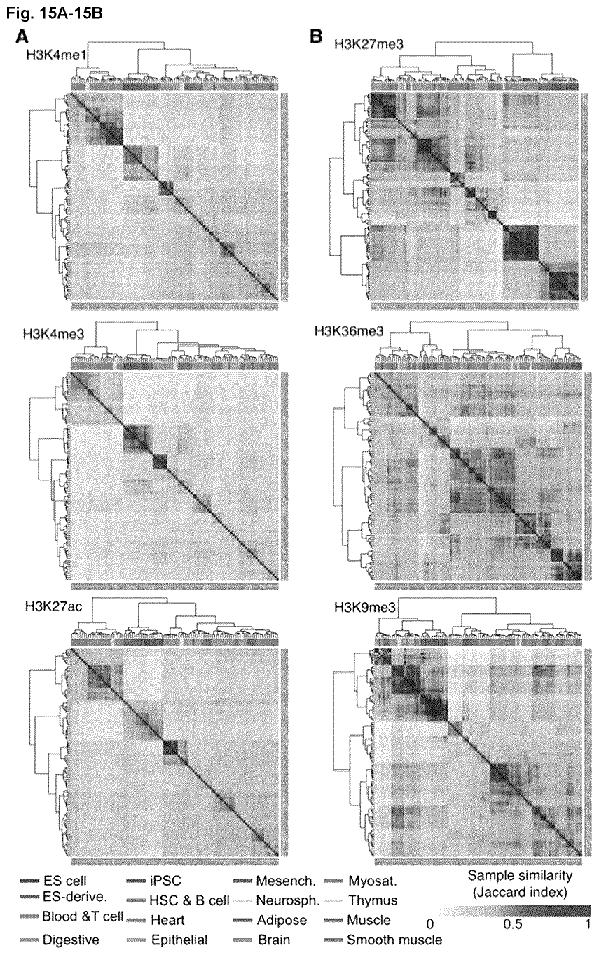

To further assess the specificity of active histone modifications in identifying primitive from differentiated cellular identity, a k nearest neighbour (k-NN) classifier was developed using LOCKs of each mark as features. Starting from the catalogue of LOCKs for each histone modification, the presence/absence of LOCKs from each histone modification within each cell type over this catalogue was assessed. This model clearly shows that LOCKs from active histone modifications stratify primitive from differentiated cell types and cluster each sample according to its tissue of origin (average Matthews Correlation Coefficient (MCC) of active marks=0.85; repressive marks=0.71) that agrees with clustering of the samples using the same similarity measure (FIG. 15).

The methods used are as described in Example 12.

### Example 10: LOCKs of Active Histone Marks Map to Genes Involved in Cell Type-Specific Biological Pathways

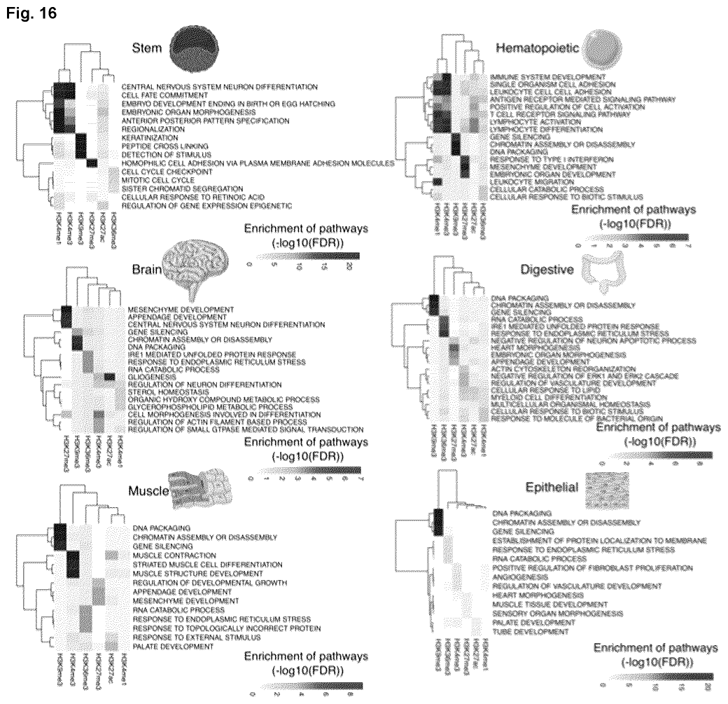

Cis-regulatory elements (CREs) defined by discrete histone modifications are important players in defining cellular identity by setting lineage-specific gene expression profiles⁶⁻¹⁰. LOCKs of active versus repressive marks were assessed to determine if they were related to pathways of relevance to ubiquitous or cell type-specific biological processes. Cell-type specific pathways showed higher enrichment among genes in proximity of LOCKs of active marks compared to LOCKs of repressive marks across all cell types (FIG. 16). For example, among the enriched pathways associated with H3K4me1 and H3K4me3 LOCKs EMBRYONIC ORGAN MORPHOGENESIS was found in stem cells, and LEUKOCYTE CELL ADHESION was found in hematopoietic cell populations (FDR<0.05) (FIG. 16). On the other hand, H3K9me3 LOCKs were enriched in proximity to genes involved in ubiquitous biological processes like GENE SILENCING across multiple tissue types (FDR<0.05) (FIG. 16).

The methods used are as described in Example 12.

### Example 11: Bivalency of LOCKs of Active Histone Marks in Stem Cells

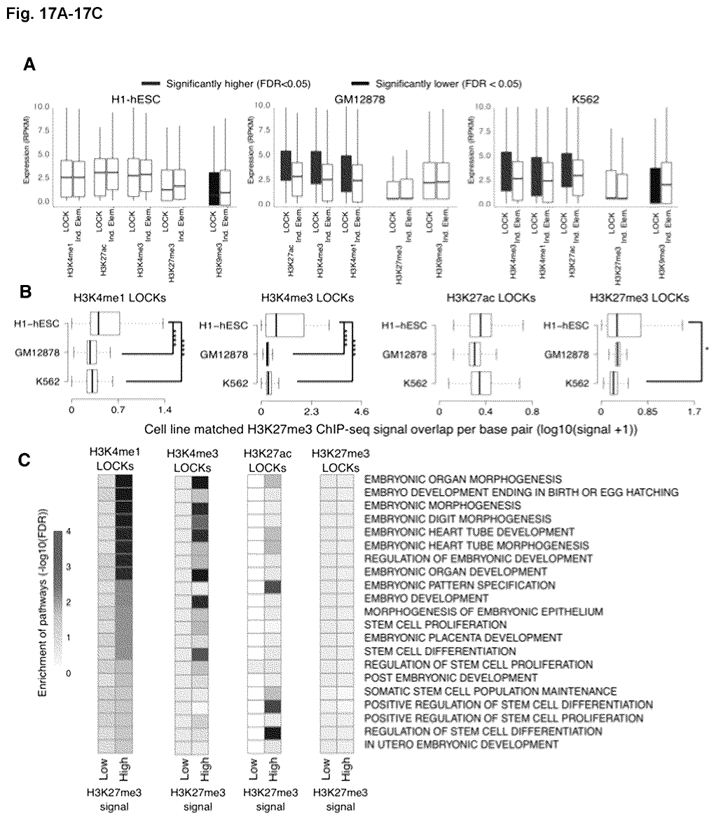

Coexistence of active and repressive histone modifications at the same loci were reported in primitive cells as bivalent chromatin states associated with genes poised for expression or repression upon cellular differentiation Hence, we assessed if bivalency is also related to LOCKs. Overlapping repressive marks signal with LOCKs from active and repressed chromatin across our collection of cell types revealed that bivalent LOCKs populate primitive cells, mapping in proximity to genes highly expressed, compared to genes in proximity of Ind. elements, only in differentiated cells, such as GM12878 and K562 as opposed to primitive H1-hESCs (FIG. 17A). The coexistence of the H3K27me3 repressive LOCKs with H3K4me1 and H3K4me3 active LOCKs was observed in primitive cells (FDR<0.05) (FIG. 17B). Notably, the H3K27me3 signal intensity did not differ within H3K27me3 LOCKs from the primitive H1-hESC versus the mature GM12878 and K562 cell types (FIG. 17B). The functional classification of genes proximal to bivalent versus active LOCKs found in the H1-hESC primitive cell type was assessed through Gene Set Enrichment Analysis. This identified an enrichment of genes proximal to bivalent LOCKs with pathways relevant to embryonic development and stem cell differentiation (FDR<0.05) (FIG. 17C). Collectively, these results suggest that bivalent LOCKs behave similarly to individual bivalent elements, populating the genome of primitive as opposed to differentiated cell types and being assigned to genes repressed in primitive cells of relevance to differentiation.

The methods used are as described in Example 12.

### Example 12: Bivalent LOCKs Populate Boundaries of Topologically Associating Domains

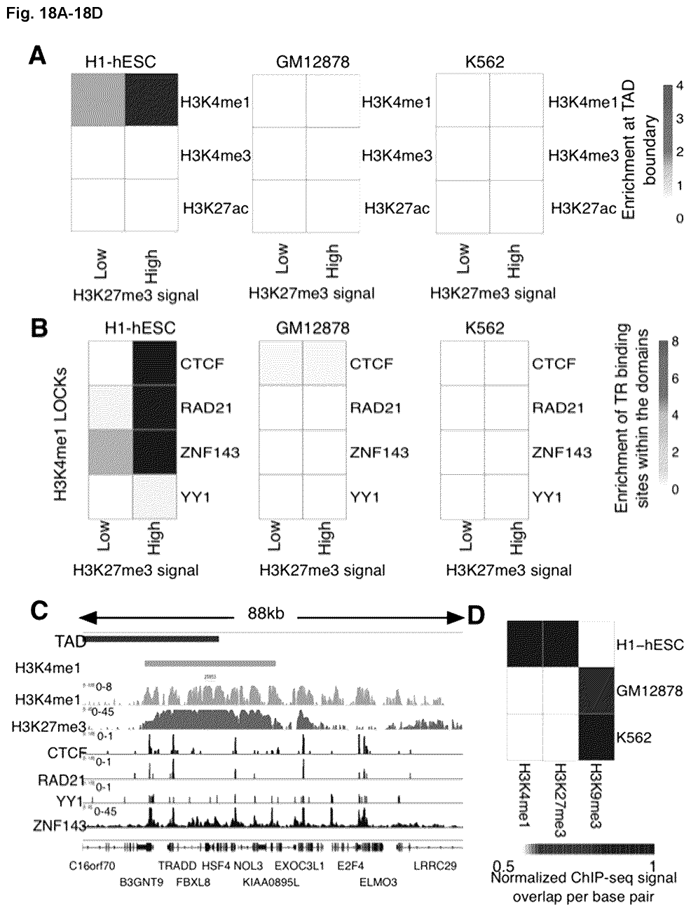

In addition to LOCKs, the genome is organized into clusters of chromatin interactions defining its three-dimensional organization^{23,24}. Clusters of chromatin interactions establish topologically associating domains (TADs)¹² that further cluster into A or B compartments according to the active versus repressed nature of the chromatin within them^{13, 25}. The three-dimensional genome organization is regulated by DNA binding proteins, namely CTCF, YY1, and ZNF143²⁰⁻²² as well as the cohesin complex^{23, 24}. Therefore the relation between LOCKs and the three-dimensional genome organization of primitive versus differentiated cells was investigated. Focusing on H1-hESC, GM12878 and K562, H3K4me1 LOCKs found in H1-hESCs were found to be enriched in proximity of TAD boundaries, while the H3K4me1 LOCKs from GM12878 and K562 did not enrich at TAD boundaries (FIG. 18A). This preferentially related to H3K4me1 LOCKs with strong H3K27me3 signal, i.e. bivalent LOCKs (FIG. 18B). H3K4me3 and H3K27ac LOCKs from primitive and differentiated cell types did not relate to TAD structures (FIG. 18A). The H3K4me1 LOCKs were further characterized with regards to the chromatin occupancy by regulators of chromatin interactions, namely CTCF, YY1, ZNF143 and the cohesin complex component RAD21. This revealed an enrichment of all regulators of chromatin interaction except YY1 at H3K4me1/H3K27me3 bivalent LOCKs from H1-hESC but not over H3K4me1 LOCKs from GM12878 and K562 cell lines (FIG. 18B), exemplified at the chromosome 16822.1 locus (FIG. 18C). Further, it was shown that the bivalent LOCKs in primitive cells transit to a repressed state in differentiated cells characterized by the gain of the H3K9me3 repressive mark and the loss of H3K4me1 and H3K27me3 (FIG. 18D). Altogether, these results suggest that changes in LOCKs composition discriminating primitive from differentiated cells occurs at TAD boundaries.

### Methods

LOCK identification: The CREAM algorithm described herein was used to identify Large Organized Chromatin Lysine (K) domains (LOCKs). CREAM is used to identify LOCKs in six steps: (1) grouping the individual elements in clusters of varying number of individual elements (referred to as Order); (2) identifying the threshold for the stitching distance between individual elements within the clusters of the same Order; (3) identifying the maximum Order of clusters (LOCKs in this case); (4) clustering individual elements as LOCKs starting from the highest Order; and (5) filtering out low Order LOCKs with a stitching distance close to the corresponding stitching distance threshold of the same Order. (6) Steps 1 to 5 are repeated until the following parameter starts large oscillations (>5%):

$$Relative sum - \frac{sum of coverage of LOCKs by individual elements}{sum of total genome coverage of LOCKs}$$

The code for identifying LOCKs is available in https://codeocean.com/capsule/6911149/tree/v1.

This approach for LOCK identification relies on identifying clusters of individual elements (peaks) identified by MACS as opposed to the ChIP-seq signal files. This limits the reported challenges in identifying broad domains for certain histone modifications from ChIP-seq signal profiles²⁷⁻²⁹.

Machine learning model for cell type classification: Similarity between two samples were identified using Jaccard index for the commonality in localization of their identified LOCKs throughout the genome. Then this Jaccard index is used as the similarity statistics in a 1-nearest-neighbor classification approach. The performance of the classification was assessed using the leave-one-out cross validation.

Association with genes: A gene is considered associated with a LOCK or individual element marked by a histone modification if found within 10 kb from each other, with an anchor on the transcription start site (TSS) for genes. This distance was chosen to avoid false-positive association of elements with gene TSSs³⁰.

Gene expression comparison: RNA sequencing profile of GM12878, K562, and H1-hESC cells lines, available in The ENCODE Project database³¹, were used to identify expression of genes in proximity of LOCKs and individual elements marked by a histone modification. Expression of genes were compared using the Wilcoxon signed-rank test.

Pathway enrichment analysis: Hypergeometric test was used to identify p-values for enrichment of gene sets using dhyper function in stats R package (version 3.5.1). LOCK-associated genes in each sample are considered as query gene sets. In case of pathway enrichment per tissue type, catalogue of genes associated with all the individual elements were used as background gene lists. For the pathway enrichment across LOCKs with different H3K27me3 signal intensity in H1-hESC, all genes in proximity of LOCKs of the same histone mark in H1-hESC are considered as background gene lists.

Assigning LOCKs to each phenotype: A LOCK is assigned to each tissue type if it exists in more than 50% of samples from that tissue type.

H3K27me3 signal intensity measurement over active LOCKs: We identified overlap of signal from bedgraph files of ChIP-Seq data of H3K27me3 with the identified LOCKs and individual elements in GM12878, K562, and H1-hESC using bedtools (version 2.23.0). The signal over LOCKs was normalized (divided by median) to the H3K27me3 ChIP-seq signal overlapped in the Ind. elements of the corresponding profiles in each cell line. The identified signal intensity within each LOCK was then further normalized to the length of the element. The distribution of the normalized scores were then compared between H1-hESC and GM12878 and K562 cell lines using Wilcoxon signed-rank test.

Enrichment of LOCKs at boundaries of topologically associating domains: TAD boundaries identified from a collection of Hi-C profiles from GM12878, K562 and H1-hESC that were processed for genome assembly GRCh37 and are available in the Hi-C browser³². LOCKs identified from ChIP-seq profiles of histone modification in a cell line were categorized to be at TAD boundaries if within 10 kb from each other. A hypergeometric test was then used to identify enrichment of LOCKs from a histone modification of a cell line with a defined H3K27me3 overlap level, low or high. The LOCKs in each category of high, low or intermediate H3K27me3 signal overlap is considered as the query set and all the LOCKs at TAD boundaries as the background.

Enrichment of binding sites for regulator of chromatin interactions at LOCKs: The number of individual binding sites for regulators of chromatin interactions within H3K4me1 LOCKs associated with low or high H3K27me3 signal from H1-hESC cells was normalized to the size of the LOCKs. The normalized binding value was then used as a query set in a hypergeometric test to measure their enrichment score. The normalized binding value of regulators of chromatin interactions over all H3K4me1 LOCKs in H1-hESC is considered as the background list within the hypergeometric test.

Multiple hypothesis correction: P-values were corrected for multiple hypothesis testing using the Benjamini-Hochberg procedure

Research reproducibility: Results of this paper can be reproduced using the cloud-based computational reproducibility platform CodeOcean (https://codeocean.com/capsule/6911149/tree/v1).

### Example 13: CREAM for Identifying Cancer Stem Cells

CREAM 200 can be used to identify enrichment of cancer stem cells within a bulk tumor sample. For example, this may be done by applying a version of method 300 shown in FIG. 1E. In this example, the method 300 first involves gathering a collection of stem cell enriched and non-stem cell enriched ATAC-seq profiles for samples across one or multiple tumor types for example, Leukaemia, Glioblastoma Multiforme (GBM), Pancreatic Adenocarcinoma and Colon Adenocarcinoma. A training set is formed from the collection of profiles of the stem cell enriched and non-stem cell enriched samples. In this example, method 300 then identifies clusters of cis-regulatory elements (COREs) for these samples using CREAM 200. Upon availability of an ATAC-seq profile of a new sample of a known tumor type (specified by user) such as a patient sample, the COREs of the new sample are identified.

Method 300 then compares the COREs of the new sample with the COREs identified for the ATAC-seq profiles of the collection of the stem cell enriched and non-stem cell enriched samples (e.g. the COGR signature standards determined in light of the stem cell and non-stem cell enriched samples). For example, the comparison can include determining the similarity of the COREs of the new sample to the COREs of the stem cell enriched and the COREs of the non-stem cell enriched samples. The similarity between the new sample and the CORE signature standard or a sample from the training set may be determined using the Jaccard similarity measure. The Jaccard similarity measure is a length of intersection of the COREs (as binary features) between the new sample and a training set sample. The size of the intersection does not matter. For example, if there are two COREs from different samples such as chr1:1-100 and chr1:40-500, there is an overlap region (chr1:40-100) and the COREs are considered as overlapping each other irrespective of how large the overlap is or how many CREs are included in the overlap (e.g. 1 base pair may be sufficient for overlap). The Jaccard similarity measure therefore determines how many COREs between the new sample and the training set sample or signature standard overlap each other divided by the length of the union of the COREs of these two samples. This operation can be repeated by comparing the COREs of the new sample to the COREs of each of the training set samples to obtain a first set of Jaccard similarity values for the COREs of the new sample and the COREs of the stem cell enriched samples and a second set of Jaccard similar values for the COREs of the new sample and the COREs of the non-stem cell enriched samples. Alternatives to the Jaccard similarity measure include, but are not limited to, the Dice dissimilarity measure, the Cosine similar measure or other similar measures as known by those skilled in the art.

The method 300 may, then determine the stem-score of the sample which is based on the difference between the similarity of the COREs of the sample to the COREs of the stem cell enriched samples and the similarity of the CORESs of the sample to the CORES of the non-stem cell enriched samples. In this example, this step of method 300 is optional.

In some embodiments, the stem-score of the sample can be determined as follows: the average of the Jaccard similarities of the new sample to cancer non-stem cell samples in the training dataset (i.e. the first set of Jaccard similarity values of step 308) will be deducted from the average of the Jaccard similarities of the new sample to the cancer stem cell samples (i.e. the second set of Jaccard similarity values of step 310). The resulting stemness score will be bounded between −1 and 1. Hence, if the stemness score is higher, it means that the new sample (i.e. a test sample) is more similar to the stem cell samples of the training set and potentially has higher stemness capacities.

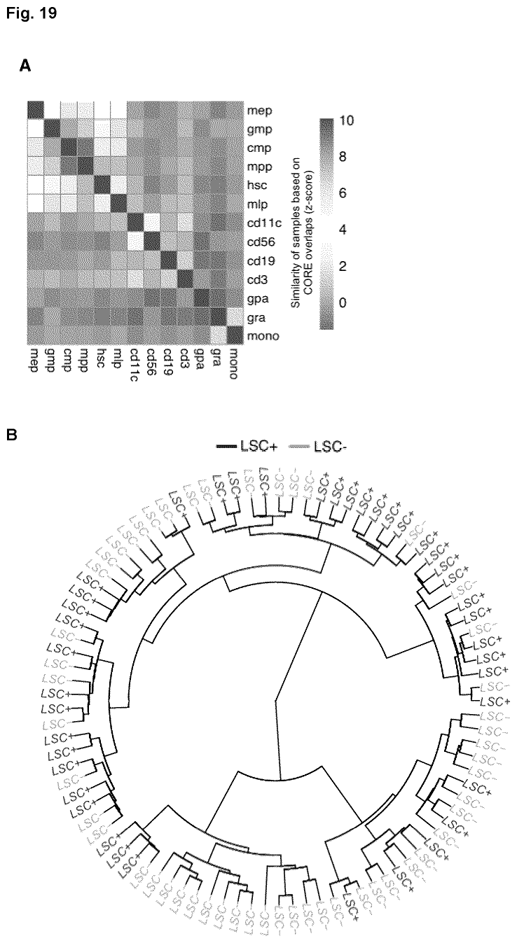

To assess if the similarities identified using COREs are a valid approach to identify the similarity of a test sample to stem and non-stem cell tissue samples, either healthy or malignant, hematopoietic and leukaemia samples were used (FIGS. 19A and B). The identified similarities resulted in clustering of hematopoietic stem cells (HSCs) and progenitor samples (MPP, CMP, GMP, MEP and MLP; FIG. 19A). Utilizing this method also resulted in clustering of Leukaemia stem cell-enriched (LSC+) samples together while separating them from differentiated (LSC−) samples (FIG. 19B). This clustering was determined using the pheatmap R package. For hematopoietic samples Ward's minimum variance method (ward.D2) is used as the clustering algorithm with the modification that dissimilarities are squared before clustering while for LSC+ and LSC− samples, the default methodologies are used. The clustering are implemented on the matrix of Jaccard similarities between samples based on the identified COREs for each sample. Hence, each element of the matrix is a Jaccard similarity between samples in the corresponding row and column.

### Example 14: CREAM for Identifying Drug Targets in Cancer Cells

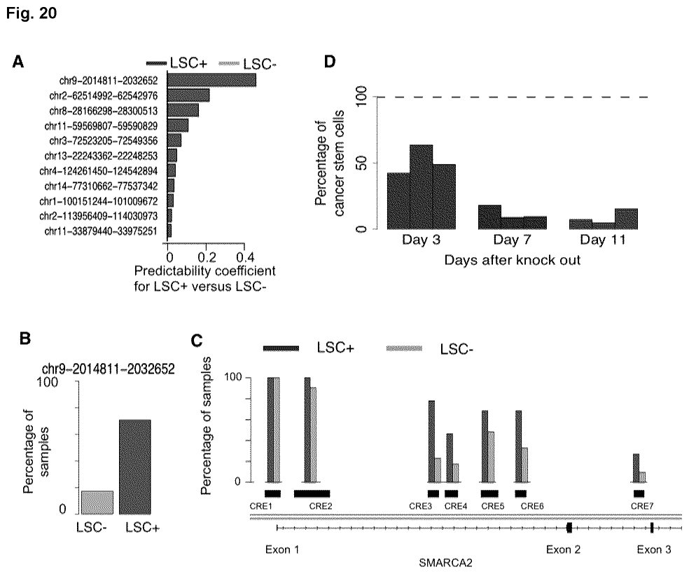

CREAM 200 can be used to identify drug targets for a phenotype of interest. For example, a version of method 350 shown in FIG. 1G can be applied in this example. For this example, the method 350 first involves obtaining the ATAC-seq profiles of cancer cells, for example leukemia stem cell enriched LSC+ and leukemia stem cell non-enriched LSC− samples, and using CREAM 200 to identify clusters of cis-regulatory elements (COREs) for each of these ATAC-seq profiles to generate a catalogue of COREs. The method 350 then identifies COREs specific to samples of one phenotype compared to another (see FIG. 20A). For example, in the present example, COREs associated with the more aggressive LSCs and which upon deletion, revert to the less aggressive LSC− phenotype may be analysed to identify genes affected by the CORE and the gene products targeted. Similarly a drug resistant cancer cell line may be compared to a non-drug resistant cancer cell line to see which COREs when deleted, revert the phenotype to drug sensitivity, may be analysed to identify genes affected by the CORE and the gene products targeted.

The method 350 then generates a feature matrix using the CORE catalogue. The feature matrix is a binary matrix of O's and Is. The rows of the feature matrix are the samples (e.g. patient tumor samples, cancer cell lines, etc.) and the columns are the features (e.g. the presence of the COREs in these samples where the COREs are from the generated catalogue). Each element of the matrix indicates where the CORE in the corresponding column exists in the samples that make up the rows of the matrix.

The method 350 then uses the features matrix and the labels of the samples being either LSC+ or LSC− to train a machine learning model. In some embodiments, the machine learning model can be an elasticnet model. In other embodiments, other sparse learning methods such as Lasso or generally any supervised learning method that either implements regularization (such as the lasso and elasticnet models) or feature selection (such as in linear discriminant analysis) can be used instead of the elasticnet model. In some embodiments, a combination of unsupervised feature selection approaches (such as principle component analysis) and any supervised machine learning (such as random forest, gradient boosting, Adaboost, etc.) can be also used.

During training of the elasticnet model, a 5-fold cross-validation was repeated 100 times to decrease a likelihood of overfitting of the model caused by each cross-validation. The cross-validation is used for evaluating the elasticnet model as this is better than using an evaluation technique based on residuals. The problem with residual evaluations is that they do not give an indication of how well the model will do when it is used to make new predictions for input data that the model has not already encountered. One way to overcome this problem is to not use the entire data set when training the model. For example, some of the data from the training data set is removed before training begins. Then when training is done, the data that was removed can be used to test the performance of the learned model on the “new” data.

The training of the elasticnet model can be implemented using the cv.glmnet function in the glmnet software package considering the binary feature matrix as the input feature matrix and an array of 0s and 1's (0 being LSC− and 1 being LSC+) as the output vector of labels. A setting of alpha=0.5 can be used as the input parameter of cv.glmnet function. If the Lasso model is used then the training can be done as explained for the elasticnet model except for using a setting of alpha=1.

The method 350 then determines COREs for identifying drug targets in cancer cells. This involved obtaining an average of the coefficients of each CORE in the catalogue across the 100 times 5-fold cross-validations. The COREs with non-zero coefficients (reported as predictability coefficients) are then determined from which the top CORE is or top COREs are identified. In the training process, irrelevant features are removed and the rest of the features are assigned a coefficient. As all of the features are binary features, e.g. either a 0 or a 1 for the non-existence or existence of COREs, and since binary classification (LSC+versus LSC− for example) is being used, the resulting coefficients can be directly used for ranking. Individual CREs within the top CORE, specific to LSC+compared to LSC− are identified. The top ranked CORE can be determined as explained for another method described herein. For example, a CRE or 2 or more CREs in the top CORE with the most difference in the number of LSC+ and LSC− samples that have them can be deleted. These knock-outs may be used to identify which genes are affected by knocking-out those CREs and then finding ligands targeting the expression products of those genes.

To test this approach, 41 LSC+ and 52 LSC− samples were used to identify drug target(s) for LSC+ phenotype (FIG. 20B). The top predicted CORE as the potential target (chr9: 2014811-2032652) existed in 71% of LSC+ while in only 17% LSC− samples (FIG. 20C). In the next step, individual cis-regulatory elements (CREs), within the top CORE, specific to LSC+ compared to LSC− are identified (CRE3 and CRE6 in FIG. 20D) CRISPR-cas9 was used to knock-out CRE3 and CRE6 as the LSC+ specific CREs within the top CORE resulting in lowering than the LSC+ percentage to less than 10% in average for the three used replicates (FIG. 20E). The identified target COREs can be used to identify potential drug targets for LSC+ phenotype.

### Example 15: CREAM for Discovering Biomarkers of Drug Response

CREAM can be used to identify biomarkers of drug response in tissue samples, animal models or cell lines relying on their ATAC-seq profiles. Upon accessibility of ATAC-seq profiles of the samples, their corresponding clusters of cis-regulatory elements (COREs) will be identified. This process will help reduce dimensionality of the dataset from >20,000 (individual cis-regulatory elements) to ˜1000 (COREs). Then the COREs will be used in binary classification models as described above to predict responders and separate them from non-responders to the tested drug (or drugs).

CREAM was tested for biomarker discovery, for drug response, using 16 breast cancer cell lines (AU565, BT549, HCC1143, HCC1395, HCC1419, HCC1806, HCC1937, HCC3153, HCC38, HCC70, MDAMB157, MDAMB361, MDAMB453, MX1, T47D, ZR751) with available ATAC-seq profiles and response to PD98059 (a non-ATP competitive MEK inhibitor), and Floxuridine (an antimetabolite agent), as part of the data available in PharmacoGx R package (version 2.0.5) developed by Haibe-Kains laboratory. There are currently no known biomarker for response to PD98059 and Floxuridine.

COREs (e.g. biomarkers) that were found to have a significant p-value (<0.05) are shown in Tables 2 and 3 for PD98059 and floxuridine respectively.

The columns in each Table are: 1) chromosome regions of the COREs; 2) pvalue; 3) Fold change.

Fold change tells you if presence of a CORE results in higher or lower sensitivity. If Fold Change is great than 1, it means that presence of a CORE results in higher sensitivity and Fold Change is less than 1, it means that presence of a CORE results in lower sensitivity.

Biomarkers indicative of response to these drugs in the 16 breast cancer cell lines (FDR<0.05) including chr11:694436-876984 and chr4:99547495-99584170 for PD98059 and Floxuridine, respectively are shown in FIG. 21.

TABLE 2
PD98059 biomarker response

| | pvalue | Fold change |
|---|---|---|
| chr11-694436-876984 | 0.00025 | 2.16958 |
| chr11-34183346-34607941 | 0.015984 | 1.85866 |
| chr15-40330702-40453457 | 0.016434 | 2.206022 |
| chr16-4965308-5008584 | 0.037918 | 1.982835 |
| chr16-53766187-53861966 | 0.010989 | 0.490151 |
| chr17-21102597-21252333 | 0.015984 | 0.524688 |
| chr19-45765981-46636866 | 0.041783 | 1.903598 |
| chr2-75602732-75966190 | 0.002098 | 0.523133 |
| chr20-9819285-10752699 | 0.007867 | 0.525321 |
| chr6-126063660-126362254 | 0.005245 | 0.432684 |
| chr7-54328557-56189467 | 0.022478 | 0.536161 |
| chr9-75681937-75835570 | 0.049883 | 0.538022 |
| chr9-103348375-103365047 | 0.041958 | 0.490151 |
| chr9-130150616-131799038 | 0.014763 | 1.905895 |
| chr9-139257512-140211180 | 0.041958 | 1.863501 |

TABLE 3
Floxuridine biomarker response

| | pvalue | Fold change |
|---|---|---|
| chr15-60619092-60725509 | 0.041958 | 0.7009 |
| chr16-29801700-30154789 | 0.010989 | 1.65259 |
| chr16-67184267-67407032 | 0.031219 | 2.016606 |
| chr17-21102597-21252333 | 0.031219 | 0.699011 |
| chr2-36473442-37039510 | 0.031219 | 0.667378 |
| chr3-69004922-69292456 | 0.020668 | 0.661685 |
| chr4-99547495-99584170 | 0.00035 | 0.540623 |
| chr5-167696005-167914094 | 0.007867 | 0.629009 |
| chr6-16577121-16782003 | 0.00025 | 0.577727 |

While the applicant's teachings described herein are in conjunction with various embodiments for illustrative purposes, it is not intended that the applicant's teachings be limited to such embodiments as the embodiments described herein are intended to be examples. On the contrary, the applicant's teachings described and illustrated herein encompass various alternatives, modifications, and equivalents, without departing from the embodiments described herein, the general scope of which is defined in the appended claims.

### REFERENCES

Bailey, Swneke D., Xiaoyang Zhang, Kinjal Desai, Malika Aid, Olivia Corradin, Richard Cowper-Sal Lari, Batool Akhtar-Zaidi, Peter C. Scacheri, Benjamin Haibe-Kains, and Mathieu Lupien. 2015. “ZNF143 Provides Sequence Specificity to Secure Chromatin Interactions at Gene Promoters.” Nature Communications 2 (February): 6186.
Boeva, Valentina, Caroline Louis-Brennetot, Agathe Peltier, Simon Durand, Cecile Pierre-Eugene, Virginie Raynal, Heather C. Etchevers, et al. 2017. “Heterogeneity of Neuroblastoma Cell Identity Defined by Transcriptional Circuitries.” Nature Genetics 49 (9): 1408-13.
Buenrostro, Jason D., Paul G. Giresi, Lisa C. Zaba, Howard Y. Chang, and William J. Greenleaf. 2013. “Transposition of Native Chromatin for Fast and Sensitive Epigenomic Profiling of Open Chromatin, DNA-Binding Proteins and Nucleosome Position.” Nature Methods, 1-8.
Chipumuro, Edmond, Eugenio Marco, Camilla L. Christensen, Nicholas Kwiatkowski, Tinghu Zhang, Clark M. Hatheway, Brian J. Abraham, et al. 2014. “CDK7 Inhibition Suppresses Super-Enhancer-Linked Oncogenic Transcription in MYCN-Driven Cancer.” Cell 159 (5): 1126-39.
Corces, M. Ryan, Jeffrey M. Granja, Shadi Shams, Bryan H. Louie, Jose A. Seoane, Wanding Zhou, Tiago C. Silva, et al. 2018. “The Chromatin Accessibility Landscape of Primary Human Cancers.” Science 362 (6413). https://doi.org/10.1126/science.aav1898.
Dowen, Jill M., Zi Peng Fan, Denes Hnisz, Gang Ren, Brian J. Abraham, Lyndon N. Zhang, Abraham S. Weintraub, et al. 2014. “Control of Cell Identity Genes Occurs in Insulated Neighborhoods in Mammalian Chromosomes.” Cell 159 (2): 374-87.
Ea, Vuthy, Marie-Odile Baudement, Annick Lesne, and Thierry Forné. 2015. “Contribution of Topological Domains and Loop Formation to 3D Chromatin Organization.” Genes 6 (3): 734-50.
ENCODE Project Consortium. 2012. “An Integrated Encyclopedia of DNA Elements in the Human Genome.” Nature 489 (7414): 57-74.
Ernst, Jason, and Manolis Kellis. 2010. “Discovery and Characterization of Chromatin States for Systematic Annotation of the Human Genome.” Nature Biotechnology 28 (8): 817-25.
Ernst, Jason, Pouya Kheradpour, Tarjei S. Mikkelsen, Noam Shoresh, Lucas D. Ward, Charles B. Epstein, Xiaolan Zhang, et al. 2011. “Mapping and Analysis of Chromatin State Dynamics in Nine Human Cell Types.” Nature 473: 43-49.
Feng, Jianxing, Tao Liu, and Yong Zhang. 2011. “Using MACS to Identify Peaks from ChIP-Seq Data.” Current Protocols in Bioinformatics/Editoral Board, Andreas D. Baxevanis . . . [et Al.] Chapter 2 (June): Unit 2.14.
Gaulton, Kyle J., Takao Nammo, Lorenzo Pasquali, Jeremy M. Simon, Paul G. Giresi, Marie P. Fogarty, Tami M. Panhuis, et al. 2010. “A Map of Open Chromatin in Human Pancreatic Islets.” Nature Publishing Group 42 (3): 255-59.
Gotea, Valer, Axel Visel, John M. Westlund, Marcelo A. Nobrega, Len A. Pennacchio, and Ivan Ovcharenko. 2010. “Homotypic Clusters of Transcription Factor Binding Sites Are a Key Component of Human Promoters and Enhancers.” Genome Research 20 (5): 565-77.
Heidari, Nastaran, Douglas H. Phanstiel, Chao He, Fabian Grubert, Fereshteh Jahanbani, Maya Kasowski, Michael Q. Zhang, and Michael P. Snyder. 2014. “Genome-Wide Map of Regulatory Interactions in the Human Genome.” Genome Research 24 (12): 1905-17.
Heintzman, Nathaniel D., Gary C. Hon, R. David Hawkins, Pouya Kheradpour, Alexander Stark, Lindsey F. Harp, Zhen Ye, et al. 2009. “Histone Modifications at Human Enhancers Reflect Global Cell-Type-Specific Gene Expression.” Nature 459: 108-12.
Heintzman, Nathaniel D., Rhona K. Stuart, Gary Hon, Yutao Fu, Christina W. Ching, R. David Hawkins, Leah O. Barrera, et al. 2007. “Distinct and Predictive Chromatin Signatures of Transcriptional Promoters and Enhancers in the Human Genome.” Nature Genetics 39 (3): 311-18.
Hnisz, Denes, Brian J. Abraham, Tong Ihn Lee, Ashley Lau, Violaine Saint-André, Alla A. Sigova, Heather A. Hoke, and Richard A. Young. 2013. “Super-Enhancers in the Control of Cell Identity and Disease.” Cell 155 (4): 934-47.
Kellis, Manolis, Barbara Wold, Michael P. Snyder, Bradley E. Bernstein, Anshul Kundaje, Georgi K. Marinov, Lucas D. Ward, et al. 2014. “Defining Functional DNA Elements in the Human Genome.” Proceedings of the National Academy of Sciences of the United States of America 111 (17): 6131-38.
Kron, Ken J., Alexander Murison, Stanley Zhou, Vincent Huang, Takafumi N. Yamaguchi, Yu-Jia Shiah, Michael Fraser, et al. 2017. “TMPRSS2-ERG Fusion Co-Opts Master Transcription Factors and Activates NOTCH Signaling in Primary Prostate Cancer.” Nature Genetics 49 (August): 1336.
Laat, Wouter de, and Frank Grosveld. 2003. “Spatial Organization of Gene Expression: The Active Chromatin Hub.” Chromosome Research: An International Journal on the Molecular, Supramolecular and Evolutionary Aspects of Chromosome Biology 11 (5): 447-59.
Liberzon, A., C. Birger, H. Thorvaldsdottir, M. Ghandi, J. P. Mesirov, and P. Tamayo. n.d. “The Molecular Signatures Database (MSigDB) Hallmark Gene Set Collection. Cell Syst. 2015; 1 (6): 417-25.” Epub 2016/01/16. https://doi. org/10.1016/j. cels. 2015.12.004 PMID: 26771021.
Lovén, Jakob, Heather A. Hoke, Charles Y. Lin, Ashley Lau, David A. Orlando, Christopher R. Vakoc, James E. Bradner, Tong Ihn Lee, and Richard A. Young. 2013. “Selective Inhibition of Tumor Oncogenes by Disruption of Super-Enhancers.” Cell 153 (2): 320-34.
Lupien, Mathieu, Jéröme Eeckhoute, Clifford A. Meyer, Qianben Wang, Yong Zhang, Wei Li, Jason S. Carroll, X. Shirley Liu, and Myles Brown. 2008. “FoxA1 Translates Epigenetic Signatures into Enhancer-Driven Lineage-Specific Transcription.” Cell 132 (6): 958-70.
McDonald, John H. 2009. Handbook of Biological Statistics. Vol. 2. sparky house publishing Baltimore, Md.
Northcott, Paul A., Catherine Lee, Thomas Zichner, Adrian M. Stutz, Serap Erkek, Daisuke Kawauchi, David J. H. Shih, et al. 2014. “Enhancer Hijacking Activates GFI1 Family Oncogenes in Medulloblastoma.” Nature 511 (7510): 428-34.
Pan, Guangjin, and James A. Thomson. 2007. “Nanog and Transcriptional Networks in Embryonic Stem Cell Pluripotency.” Cell Research 17 (1): 42-49.
Rao, Suhas S. P., Miriam H. Huntley, Neva C. Durand, Elena K. Stamenova, Ivan D. Bochkov, James T. Robinson, Adrian L. Sanborn, et al. 2014. “A 3D Map of the Human Genome at Kilobase Resolution Reveals Principles of Chromatin Looping.” Cell 159 (7): 1665-80.
Ren, Ruibao. 2005. “Mechanisms of BCR-ABL in the Pathogenesis of Chronic Myelogenous Leukaemia.” Nature Reviews. Cancer 5 (3): 172-83.
Sanyal, Amartya, Bryan R. Lajoie, GauravJain, and Job Dekker. 2012. “The Long-Range Interaction Landscape of Gene Promoters.” Nature 489 (7414): 109-13.
Shankar, Deepa B., Jerry C. Cheng, Kentaro Kinjo, Noah Federman, Theodore B. Moore, Amandip Gill, Nagesh P. Rao, Elliot M. Landaw, and Kathleen M. Sakamoto. 2005. “The Role of CREB as a Proto-Oncogene in Hematopoiesis and in Acute Myeloid Leukemia.” Cancer Cell 7 (4): 351-62.
Smirnov, Petr, Zhaleh Safikhani, Nehme El-Hachem, Dong Wang, Adrian She, Catharina Olsen, Mark Freeman, et al. 2016. “PharmacoGx: An R Package for Analysis of Large Pharmacogenomic Datasets.” Bioinformatics 32 (8): 1244-46.
Somasundaram, Rajesh, Mahadesh A. J. Prasad, Jonas Ungerback, and Mikael Sigvardsson. 2015. “Transcription Factor Networks in B-Cell Differentiation Link Development to Acute Lymphoid Leukemia.” Blood 126 (2): 144-52.
Song, L., Z. Zhang, L. L. Grasfeder, A. P. Boyle, P. G. Giresi, B. K. Lee, N. C. Sheffield, et al. 2011. “Open Chromatin Defined by DNaseI and FAIRE Identifies Regulatory Elements That Shape Cell-Type Identity.” Genome Research 21 (10): 1757-67.
Thurman, Robert E., Eric Rynes, Richard Humbert, Jeff Vierstra, Matthew T. Maurano, Eric Haugen, Nathan C. Sheffield, et al. 2012. “The Accessible Chromatin Landscape of the Human Genome.” Nature 489 (7414): 75-82.
Vahedi, Golnaz, Yuka Kanno, Yasuko Furumoto, Kan Jiang, Stephen C. J. Parker, Michael R. Erdos, Sean R. Davis, et al. 2015. “Super-Enhancers Delineate Disease-Associated Regulatory Nodes in T Cells.” Nature 520 (7548): 558-62.
Wang, Tim, Kivanç Birsoy, Nicholas W. Hughes, Kevin M. Krupczak, Yorick Post, Jenny J. Wei, Eric S. Lander, and David M. Sabatini. 2015. “Identification and Characterization of Essential Genes in the Human Genome.” Science 350 (6264): 1096-1101.
Weintraub, Abraham S., Charles H. Li, Alicia V. Zamudio, Alla A. Sigova, Nancy M. Hannett, Daniel S. Day, Brian J. Abraham, et al. 2017. “YY1 Is a Structural Regulator of Enhancer-Promoter Loops.” Cell 171 (7): 1573-88.e28.
Whyte, Warren A., David A. Orlando, Denes Hnisz, Brian J. Abraham, Charles Y. Lin, Michael H. Kagey, Peter B. Rahl, Tong Ihn Lee, and Richard A. Young. 2013. “Master Transcription Factors and Mediator Establish Super-Enhancers at Key Cell Identity Genes.” Cell 153 (2): 307-19.
Yang, Zhong-Fa, Haojian Zhang, Leyuan Ma, Cong Peng, Yaoyu Chen, Junling Wang, Michael R. Green, Shaoguang Li, and Alan G. Rosmarin. 2013. “GABP Transcription Factor Is Required for Development of Chronic Myelogenous Leukemia via Its Control of PRKD2.” Proceedings of the National Academy of Sciences of the United States of America 110 (6): 2312-17.
1. Frankish, A. et al. GENCODE reference annotation for the human and mouse genomes. Nucleic Acids Res. 47, D766-D773 (2019).
2. Bourque, G. et al. Ten things you should know about transposable elements. Genome Biol. 19, 199 (2018).
3. Dekker, J. & Mirny, L. The 3D Genome as Moderator of Chromosomal Communication. Cell 164, 1110-1121 (2016).
4. Guelen, L. et al. Domain organization of human chromosomes revealed by mapping of nuclear lamina interactions. Nature 453, 948-951 (2008).
5. Sima, J. et al. Identifying cis Elements for Spatiotemporal Control of Mammalian DNA Replication. Cell 176, 816-830.e18 (2019).
6. Roadmap Epigenomics Consortium et al. Integrative analysis of 111 reference human epigenomes. Nature 518, 317-330 (2015).
7. Ernst, J. et al. Mapping and analysis of chromatin state dynamics in nine human cell types. Nature 473, 43-49 (2011).
8. Heintzman, N. D. et al. Histone modifications at human enhancers reflect global cell-type-specific gene expression. Nature 459, 108-112 (2009).
9. Lupien, M. et al. FoxA1 translates epigenetic signatures into enhancer-driven lineage-specific transcription. Cell 132, 958-970 (2008).
10. Heintzman, N. D. et al. Distinct and predictive chromatin signatures of transcriptional promoters and enhancers in the human genome. Nat. Genet. 39, 311-318 (2007).
11. van Steensel, B. & Belmont, A. S. Lamina-Associated Domains: Links with Chromosome Architecture, Heterochromatin, and Gene Repression. Cell 169, 780-791 (2017).
12. Dixon, J. R. et al. Topological domains in mammalian genomes identified by analysis of chromatin interactions. Nature 485, 376-380 (2012).
13. Pope, B. D. et al. Topologically associating domains are stable units of replication-timing regulation. Nature 515, 402-405 (2014).
14. Tonekaboni, S. A. M., Mazrooei, P., Kofia, V., Haibe-Kains, B. & Lupien, M. Identifying clusters of cis-regulatory elements underpinning TAD structures and lineage-specific regulatory networks. Genome Research vol. 29 1733-1743 (2019).
15. Wen, B., Wu, H., Shinkai, Y., Irizarry, R. A. & Feinberg, A. P. Large histone H3 lysine 9 dimethylated chromatin blocks distinguish differentiated from embryonic stem cells. Nat. Genet. 41, 246-250 (2009).
16. Deblois, G. et al. Metabolic adaptations underlie epigenetic vulnerabilities in chemoresistant breast cancer. BioRxiv (2018): 286054, doi:10.1101/286054.
17. Zhu, J. et al. Genome-wide chromatin state transitions associated with developmental and environmental cues. Cell 152, 642-654 (2013).
18. Bernstein, B. E. et al. A bivalent chromatin structure marks key developmental genes in embryonic stem cells. Cell 125, 315-326 (2006).
19. Stergachis, A. B. et al. Developmental fate and cellular maturity encoded in human regulatory DNA landscapes. Cell 154, 888-903 (2013).
20. Gómez-Diaz, E. & Corces, V. G. Architectural proteins: regulators of 3D genome organization in cell fate. Trends Cell Biol. 24, 703-711 (2014).
21. Ong, C.-T. & Corces, V. G. CTCF: an architectural protein bridging genome topology and function. Nature Reviews Genetics vol. 15 234-246 (2014).
22. Bailey, S. D. et al. ZNF143 provides sequence specificity to secure chromatin interactions at gene promoters. Nature Communications vol. 6 (2015).
23. Zuin, J. et al. Cohesin and CTCF differentially affect chromatin architecture and gene expression in human cells. Proc. Natl. Acad. Sci. U.S.A 111, 996-1001 (2014).
24. Rowley, M. J. & Corces, V. G. Organizational principles of 3D genome architecture. Nat. Rev. Genet. 19, 789-800 (2018).
25. Bouwman, B. A. M. & de Laat, W. Getting the genome in shape: the formation of loops, domains and compartments. Genome Biol. 16, 154 (2015).
26. Zhang, Y. et al. Model-based analysis of ChIP-Seq (MACS). Genome Biol. 9, R137 (2008).
27. Filion, G. J. & van Steensel, B. Reassessing the abundance of H3K9me2 chromatin domains in embryonic stem cells. Nature genetics vol. 42 4; author reply 5-6 (2010).
28. Hawkins, R. D. et al. Distinct epigenomic landscapes of pluripotent and lineage-committed human cells. Cell Stem Cell 6, 479-491 (2010).
29. Lienert, F. et al. Genomic prevalence of heterochromatic H3K9me2 and transcription do not discriminate pluripotent from terminally differentiated cells. PLoS Genet. 7, e1002090 (2011).
30. Sanyal, A., Lajoie, B. R., Jain, G. & Dekker, J. The long-range interaction landscape of gene promoters. Nature 489, 109-113 (2012).
31. ENCODE Project Consortium. An integrated encyclopedia of DNA elements in the human genome. Nature 489, 57-74 (2012).
32. Wang, Y. et al. The 3D Genome Browser: a web-based browser for visualizing 3D genome organization and long-range chromatin interactions. Genome Biol. 19, 151 (2018).
33. McDonald, J. H. Handbook of biological statistics. vol. 2 (sparky house publishing Baltimore, Md., 2009).

1. A method of determining a clusters of genomic regions (COGR) signature, optionally a Clusters of cis-Regulatory Elements (CORE) signature in a biological sample, the method comprising:
obtaining a chromatin profile of genomic DNA of the biological sample;
identifying two or more individual cis-regulatory elements (CREs) in the chromatin profile; and
locating one or more COGRs optionally COREs, to generate a COGR signature, optionally a CORE signature, comprising the steps of:
i) grouping different numbers of neighboring individual CREs throughout the genome and categorizing the groups according to order (O) which is the number of neighboring individual CREs in the groups;
ii) identifying a maximum window size (MWS) by estimating a distribution of window sizes for each order O based on a maximum distance between the individual CREs in all groups of that order O within the chromatin profile and calculating the MWS according to the following:
a) MWS=Q1(log(WS))−1.5*IQ(log(WS)), where Q1(log(WS)) and IQ(log(WS)) are first quartile and interquartile distributions of window sizes of that order O, respectively; or
b) MWS=Q1(log(WS_{normalized}))−1.5*IQ(log(WS_{normalized})) where
$$W {S}_{normalized} = max ( \frac{\begin{matrix}distance between two consecutive \\ elements in the group of CREs\end{matrix}}{\begin{matrix}average size of the two consecutive \\ elements in the group of CREs\end{matrix}} ) ;$$
identifying a maximum O (Omax), defined as a value of given order O at which an increase in the given order O does not result in an increase in the MWS for the given order O;
iii) identifying potential COGRs, optionally potential COREs, by calling groups of CREs of a particular order O with a window size less than the MWS for the particular order O as the potential COGRs, optionally the potential COREs, for each order O from Omax to O=2; and
iv) for each order O from O=2 to Omax, calculating the change in (MWS-median(WS))/median(WS), where WS is a distribution of maximum distance between individual CREs within the potential COGRs, optionally the potential COREs, of that order and filtering out lower order potential COGRs, optionally potential CORES up to a point where (MWS-median(WS))/median(WS) decreases with increasing order O, and any remaining potential COGRs, optionally potential COREs, are identified as actual COGRs, optionally actual COREs, and included in the COGR signature, optionally the CORE signature.

2. The method of claim 1, wherein the chromatin profile is a chromatin accessibility profile, optionally an ATAC-seq, a DNAse-seq, or a Faire-seq, and the COGR signature is a CORE signature, or wherein the chromatin profile is a histone modification profile, optionally ChIP-seq profiles of a histone modification selected from H3K4me1, H3K4me3, H3K27ac, H3K9me3, H3K27me3 and H3K36me3 and the COGR signature a LOCKs signature, the method further comprising repeating steps i) to v) until the parameter defined by the equation
$$Relative sum - \frac{sum of coverage of LOCKs by individual elements}{sum of total genome coverage of LOCKs}$$
starts oscillations of >5%.

3. (canceled)

4. The method of claim 1, wherein the method is performed for a plurality of biological samples, each of the biological samples having an associated or determined phenotype of a plurality of phenotypes, the method further comprising identifying one or more COGRs of the COGR signatures to be associated with one of the phenotypes of the plurality of phenotypes, the one or more COGRs thereby providing one or more of COGR signature standards, optionally wherein the identifying one or more COGRs of the COGR signatures comprises:
assessing each COGR of each COGR signature to build a univariate predictive model of outcome;
ranking each COGR according to a phenotype prediction, and
optionally generating a multivariate prediction model using two or more ranked COGRs,
wherein one or more of the identified COGRs with phenotype prediction above a selected threshold is selected to provide the COGR signature standard.

5. (canceled)

6. The method of claim 1, further comprising identifying genomic structure and/or one or more genes, optionally gene transcriptional start sites, within 25 kb upstream or downstream of one or more of the COGRs of the COGR signature or a CRE within the COGR.

7. A method of determining a cell or tissue of origin and/or a differentiation state of cells of a biological sample, the method comprising determining a COGR signature, optionally a CORE signature, of the biological sample according to claim 1 comparing the COGR signature, optionally the CORE signature, to one or more COGR signature standards, optionally one or more CORE signature standards, each derived from a plurality of cells or tissues of known origin and/or differentiation states, and identifying the cell or tissue or origin and/or the differentiation state of the biological sample according to the COGR signature standard most similar to the COGR signature.

8. (canceled)

9. A method of for identifying a biomarker associated with a selected phenotype, the method comprising:
determining a COGR signature according to claim 1, optionally a CORE signature, for a plurality of biological samples, each of the biological samples having an associated or determined phenotype for one of a plurality of phenotypes;
assessing each COGR of each COGR signature to build a univariate predictive model of outcome;
ranking each COGR according a phenotype prediction, and
optionally generating a multivariate prediction model using two or more ranked COGRs;
wherein one or more of the COGRs of the COGR signature with phenotype prediction above a selected threshold is the identified phenotype biomarker and optionally is included in a COGR signature standard optionally wherein the phenotype is drug sensitivity, stemness of a biological sample, or enrichment for stem cells in a tumor sample.

10. (canceled)

11. A method for identifying if a tumor sample is enriched for cancer stem cells, the method comprising
determining a COGR signature of a biological sample of known tumor type, according to claim 1;
assessing a similarity of the COGR signature of the biological sample with at least one COGR signature standard of a tumor sample known to be stem cell enriched and at least one COGR signature standard of a tumor sample known not to be stem cell enriched;
determining any difference between the similarity to at least one COGR signature standard known to be stem cell enriched and the at least one COGR signature standard known not to be stem cell enriched; and
assigning a score indicative of the stemness of the biological sample
optionally wherein the stemness score is assigned by determining an average Jaccard similarity of at least one stem cell enriched COGR signature standard (A) and an average Jaccard similarity of the at least one non-stem cell enriched COGR signature standard (B), wherein the stemness score is A-B.

12. (canceled)

13. (canceled)

14. A method of identifying a drug target, the method comprising:
determining a COGR signature according to claim 1, particularly a CORE signature, for a plurality of biological samples, each of the biological samples having an associated or determined phenotype for one of a plurality of phenotypes, optionally two phenotypes, optionally the two phenotypes including leukemia stem cells+ and leukemia stem cells−;
assessing each COGR of each COGR signature to build a predictive model of each phenotype;
ranking each COGR according to phenotype prediction to identify top COGRs,
identifying individual CREs within one or more of the top COGRs that are specific for a selected phenotype of the plurality of phenotypes,
genetically deleting one or more of the individual CREs in cells having the selected phenotype,
determining a COGR signature according to claim 1, for the genetically deleted cells and assessing if the genetically deleted cells have a non-selected phenotype; and
identifying one or more of the ranked COREs as potential drug targets if the COGR signature of the genetically deleted cells is more similar to a non-selected phenotype than the selected phenotype.

15. The method of claim 14, wherein the method further comprises identifying individual CREs in one or more of the ranked COREs specific to the selected phenotype, genetically modifying one or more of the individual CREs in a cell population corresponding to the selected phenotype and assessing if the genetic modification changes the cell population so that it is more similar to the non-selected phenotype than the selected phenotype.

16. A method for identifying a prognostic biomarker, the method comprising:
determining a COGR signature according to claim 1, particularly a CORE signature, for each of a plurality of tumor samples of a tumor type, each of the tumor samples having associated outcome data;
assessing each COGR of each COGR signature to build a univariate predictive model of outcome;
ranking each COGR according to a risk association prediction, and
optionally generating a multivariate prediction model using two or more ranked COGRs, wherein one or more of the identified COGRs with risk prediction above a selected threshold is the prognostic biomarker, and optionally provides a COGR signature standard.

17. A method of determining the prognosis of a patient diagnosed with a cancer, the method comprising determining a COGR signature, optionally a CORE signature, of a biological sample previously acquired from the patient according to claim 1, comparing the COGR signature, optionally the CORE signature of the biological sample to one or more COGR signature standards, optionally CORE signature standards, associated with an outcome, and providing the patient with a prognosis according to the associated outcome of the COGR signature standard, optionally the CORE signature standard, with a greatest similarity to the COGR signature of the biological sample.

18. (canceled)

19. (canceled)

20. The method of claim 17, wherein the patient has been diagnosed with lung adenocarcinoma, and the CORE signature comprises chr10:14532486-14612373 and/or chr12:56751131-56775057 and the prognosis of the patient is determined to be poor, optionally a three year survival rate of zero when a CORE is detected at chr10:14532486-14612373 or chr12:56751131-56775057 and good when said CORE is not detected in the CORE signature, optionally wherein the prognosis is determined to be very poor, optionally a one year survival rate of zero when a CORE is detected at both chr10:14532486-14612373 and chr12:56751131-56775057 and good when said COREs are not both detected in the CORE signature.

21. (canceled)

22. The method of claim 17, wherein the patient has been diagnosed with colon adenocarcinoma and the CORE signature comprises one or more of the following chromosomal locations:
a) chr14:55050826-55052359;
b) chr10:93417251-93483337;
c) chr12:27243328-27348144;
d) chr13:27169061-27175204;
e) chr17:28318276-28385918;
f) chr19:43518020-43535912; or
g) chr2:74548755-74577205.

23. The method of claim 22, wherein the prognosis is determined to be poor, optionally a five year survival rate of zero when a CORE is identified at least one of a) orb) and good when said CORE is not detected in the CORE signature, or wherein the prognosis is determined to be poor, optionally a four year survival rate of zero when a CORE is identified at two or more of a) through g), optionally, a CORE is identified at: a) and c); c) and e); d) and e); or f) and g) and good when said COREs are not detected in the CORE signature, or wherein the prognosis is determined to be very poor, optionally a two year survival rate of zero when a CORE is identified at three or more of a) through g), optionally a CORE is identified at: a), b), c), d), e) and f); a), d), e) and f); a), b), c), f) and q); or b), c), d), e) and g) and good when said COREs are not detected in the CORE signature.

24. (canceled)

25. (canceled)

26. The method of claim 17, wherein
(I) the patient has been diagnosed with kidney renal papillary cell carcinoma, and the CORE signature standard two or more comprises chromosomal locations:
a) chr6_43469265_43523300;
b) chr1_192805761_192814841;
c) chr1_227944063_227949006;
d) chr12_57450441_57463071;
e) chr16_70379118_70382380;
f) chr17_63768370_63786036;
g) chr19_5677033_5721143;
h) chr20_46346737_46368831;
i) chr8_96260662_96268917;
j) chr8_141338513_141447436; and/or
k) chrX_143632935_143636339
wherein the prognosis of the patient is determined to be good if a CORE is identified at more than two of the chromosomal locations;
(II) the patient has been diagnosed with stomach adenocarcinoma, and the CORE signature standard comprises one or more of the chromosomal locations:
a. chr13_113140862_113217081;
b. chr2_237858279_237905713; and/or
c. chr20_3845512_3847548
wherein the prognosis of the patient is determined to be very poor, optionally a one year survival rate of zero, when a CORE is identified at only one or none of the chromosomal locations in the CORE signature;
(III) wherein the patient has been diagnosed with liver hepatocellular carcinoma, and the CORE signature standard comprises one or more of the following chromosomal locations:
a) chr1:167600169-167606030;
b) chr1:235646218-235651336;
c) chr10:59173772-59182122;
d) chr11:121652910-121657125;
e) chr2:102020545-102071073;
f) chr20:19958226-20019574;
g) chr5:10351451-10355436;
h) chr5:60699338-60700881;
i) chr5:75034328-75056067;
j) chr5:78636611-78649820;
k) chr5:78971924-78986100;
l) chr6:28349674-28357446; and
m) chr8:49909523-49924044
wherein the prognosis is determined to be very poor, optionally a two year survival rate of zero, when a CORE is identified at one or more of the chromosomal locations in the CORE signature; or
(IV) the patient has been diagnosed with lung squamous cell carcinoma, and the CORE signature standard comprises at one or more of the following chromosomal locations:
a) chr19:17605694-17607218; and
b) chr1:113388501-113394601
wherein prognosis of the patient is determined to be poor, optionally a three year survival rate of zero, when a CORE is identified at one or more of the chromosomal locations in the CORE signature.

27. (canceled)

28. (canceled)

29. (canceled)

30. A method of selecting a treatment for a patient with cancer, the method comprising:
determining a COGR signature of a biological sample previously acquired from the patient according to claim 1;
comparing the COGR signature to one or more COGR signature standards having an associated drug sensitivity; and
selecting a treatment according to the associated drug sensitivity of the COGR signature standard with the greatest similarity;
optionally wherein the COGR signature is a COGR signature standard comprising a CORE in Table 2 or 3.

31. (canceled)

32. The method of claim 30, wherein the subject has breast cancer and
(I) wherein the method comprises
determining a CORE signature of a biological sample previously acquired from the patient according to claim 1,
comparing the CORE signature to one or more CORE signature standards having an associated drug sensitivity, the CORE signature standard for PD98059 drug sensitivity comprising a biomarker in Table 2 and the CORE signature standard for floxuridine drug sensitivity comprising a biomarker in Table 3, and
selecting a treatment according to the associated drug sensitivity of the CORE signature standard with the greatest similarity;
(II) wherein if a patient is identified to have a CORE signature comprising one or more of chr11-694436-876984, chr11-34183346-34607941, chr15-40330702-40453457, chr16-4965308-5008584, chr19-45765981-46636866, chr9-130150616-131799038 and/or chr9-139257512-140211180, a treatment comprising MEK inhibitor optionally PD98059 is selected and wherein if a patient is identified to have a CORE signature comprising one or more of chr16-53766187-53861966, chr17-21102597-21252333, chr2-75602732-75966190, chr20-9819285-10752699, chr6-126063660-126362254, chr7-54328557-56189467, chr9-75681937-75835570 and/or chr9-103348375-103365047, a treatment lacking MEK inhibitor such as PD98059 is selected; or
(III) wherein if a patient is identified as having a CORE signature comprising one or more of chr16-29801700-30154789 and/or chr16-67184267-67407032 a treatment comprising a pyrimidine analogue optionally floxuridine is selected and wherein if a patient is identified as having a CORE signature comprising one or more of chr15-60619092-60725509, chr17-21102597-21252333, chr2-36473442-37039510, chr3-69004922-69292456, chr4-99547495-99584170, chr5-167696005-167914094 and/or chr6-16577121-16782003 a treatment lacking a pyrimidine analogue optionally floxuridine is selected.

33. (canceled)

34. (canceled)

35. (canceled)

36. A method of assessing if a patient is likely to respond to a treatment comprising PD98059 or Floxuridine, the method comprising:
determining a CORE signature of a biological sample previously acquired from the patient according to claim 1, wherein the biological sample is a breast cancer sample, comparing the CORE signature to one or more CORE signature standards having an associated drug sensitivity, the CORE signature standard for PD98059 drug sensitivity comprising a biomarker in Table 2, optionally chr11:694436-876984 and the CORE signature standard for floxuridine drug sensitivity comprising a biomarker in Table 3 optionally chr4:99547495-99584170 and identifying the patient outcome according to the associated drug sensitivity of the CORE signature standard with the greatest similarity.

37. A method of monitoring disease progression, the method comprising:
determining a first COGR signature, optionally a first CORE signature, of a biological sample previously acquired from the patient according to claim 1,
determining a subsequent COGR signature, optionally a subsequent CORE signature, of a subsequent biological sample previously acquired from the patient according to claim 1;
comparing the first COGR signature and the subsequent COGR signature and one or more COGR signature standards each associated with an outcome, and
determining if the subsequent signature COGR signature is the same or more similar to a good outcome COGR signature standard than is the first COGR signature, indicating a lack of progression or determining if the first signature COGR signature is the same or more similar to a good outcome COGR signature standard than is the subsequent COGR signature, indicating disease progression.

38. The method of claim 37, wherein the subsequent biological sample is obtained after the patient has started treatment, and/or further comprises treating the patient or changing the treatment, if the patient is progressing.

39. (canceled)

40. A system comprising:
a memory having program code stored thereon; and
a processor that is operatively coupled to the memory, wherein the processor is configured to perform one or more methods defined according to claim 1 when at least some of the program code is executed by the processor.

41. A computer readable medium having program code stored thereon that configures a processor, when executing at least some of the program code, to perform one or more methods defined according to claim 1.
